(19) United States
(12) Patent Application Publication
(10) Pub. No.: US 2021/0275646 A1
(43) Pub. Date: Sep. 9, 2021

(54) PHARMACOLOGIC TREATMENT FOR RIGHT VENTRICULAR FAILURE

(71) Applicant: The Trustees of Columbia University in the City of New York, New York, NY (US)

(72) Inventors: Emily Joy Tsai, New York, NY (US); Christos Tzimas, New York, NY (US)

(21) Appl. No.: 17/245,531

(22) Filed: Apr. 30, 2021

Related U.S. Application Data
(63) Continuation of application No. PCT/US19/59519, filed on Nov. 1, 2019.
(60) Provisional application No. 62/836,315, filed on Apr. 19, 2019.
(60) Provisional application No. 62/755,106, filed on Nov. 2, 2018.

(51) Int. Cl. A61K 38/46; A61K 31/7088; A61P 9/04; A61K 35/761

(52) U.S. Cl. CPC A61K 38/465; A61K 35/761; A61P 9/04; A61K 31/7088

(57) ABSTRACT

The present disclosure provides, inter alia, methods for treating or ameliorating the effect of a cardiopulmonary disease, including right ventricular failure (RVF), in a subject. Also provided are methods for diagnosing the risk of having RVF in a subject, methods for preventing RVF in a subject, methods for preventing non-canonical autophagy, methods for mitigating oxidative stress in mitochondria of a cell, and methods for inhibiting microtubule-mediated active mRNA transfer in a cell. A pharmaceutical composition and treatment methods using such composition are also provided.

### CROSS REFERENCE TO RELATED APPLICATIONS

The present application is a continuation of PCT international application no. PCT/US2019/059519, filed on Nov. 1, 2019, which claims benefit of U.S. Provisional Patent Application Ser. No. 62/755,106, filed on Nov. 2, 2018, and U.S. Provisional Patent Application Ser. No. 62/836,315, filed on Apr. 19, 2019, which applications are incorporated by reference herein in their entireties.

### GOVERNMENT FUNDING

This invention was made with government support under grant nos. HL109159, HL133706 and HL138528, awarded by the National Institutes of Health. The government has certain rights in the invention.

### FIELD OF DISCLOSURE

The present disclosure provides, inter alia, methods for treating or ameliorating the effect of a cardiopulmonary disease, including right ventricular failure (RVF) in a subject. Also provided are methods for diagnosing the risk of having RVF in a subject.

### INCORPORATION BY REFERENCE OF SEQUENCE LISTING

This application contains references to amino acids and/or nucleic acid sequences that have been filed concurrently herewith as sequence listing text file “CU18328-seq.txt”, file size of 7 KB, created on Oct. 10, 2019. The aforementioned sequence listing is hereby incorporated by reference in its entirety pursuant to 37 C.F.R. § 1.52(e)(5).

### BACKGROUND OF THE DISCLOSURE

Right ventricular dysfunction (RVD) is highly prevalent and predicts worse clinical outcomes, including death, in heart failure (HF) patients (Blauwet et al. 2016; Campbell et al. 2013; Cenkerova et al. 2015; Dini et al. 2007; Drazner et al. 2013; Gerges et al. 2015; Ghio et al. 2013; Gulati et al. 2013; Kim et al. 2012; Kusunose et al. 2016; Olson et al. 2012; Park et al. 2015) and pulmonary hypertension (PH) patients (Forfia et al. 2006; Grapsa et al. 2015; Haddad et al. 2015; van de Veerdonk et al. 2011), irrespective of their etiology, left ventricular function, and pulmonary artery pressures. More than half of all HF patients are estimated to have RVD—25-50% of HF patients with preserved left ventricular ejection fraction (HFpEF) and up to 75% of those with reduced left ventricular ejection fraction (HFrEF) (Gulati et al. 2013; Damy et al. 2012; Gorter et al. 2016; Mohammed et al. 2014; Puwanant et al. 2009). The prevalence of RVD amongst PH patients varies with the cause of PH, ranging from 20-60% of those who survive pulmonary embolism (Ribeiro et al. 1999; Sista et al. 2017), to two-thirds of patients with advanced airway or parenchymal lung disease (Collum et al. 2017; Kolb et al. 2012; Vizza et al. 1998), to nearly all patients with advanced pulmonary vascular disease (Vizza et al. 1998). In the past decade, as technological advances in cardiac imaging have provided non-invasive alternatives to RV functional assessment by right heart catheterization (Rudski et al. 2010; Swift et al. 2012), more clinical studies have incorporated RV functional assessment, and the significance of RVD on the morbidity and mortality of cardiopulmonary diseases has become overwhelmingly apparent (Ghio et al. 2013; Kato et al. 2013; Courand et al. 2015; Konstantinides et al. 2017; McLaughlin et al. 2004; Gall et al. 2017).

Strikingly, current evidence-based guideline directed HF pharmacotherapies (Yancy et al. 2013) neither reverse RVD nor prevent RVF. This discrepancy should not be surprising. The right ventricle is distinct from the left ventricle with regards to embryological origin, morphology, physiology, and response to stress (Haddad et al. 2008; Haddad et al. 2008; Zaffran et al. 2004). At present, diuretics, inotropic medications, vasopressors, and, in select cases, advanced pulmonary vasodilators are the empiric pharmacotherapies used for managing RVF. However, none of these improve outcomes in RVF and there remain no RV-targeted therapies for either PH or HF patients. Hence, as appreciation for the prevalence and significance of RVD has grown, so has the urgency to identify novel therapeutic targets for RVF.

To date, efforts in novel drug development for PH and HF have respectively focused on pulmonary vascular remodeling and LV function, not RV function. Preclinical studies have been challenged by limitations of available and commonly used animal models. A few groups have sought to identify molecular signatures of RVF, using animal models (Drake et al. 2011; Gao et al. 2006; Reddy et al. 2013; Urashima et al. 2008) or even human tissue (di Salvo et al. 2015; di Salvo et al. 2015; Williams et al. 2018). Prior transcriptomic animal studies, however, were limited by models that stressed the RV without definitively inducing RVF (Drake et al. 2011; Gao et al. 2006; Reddy et al. 2013) or compared an RVF model to one of pathological LV remodeling rather than LV failure (Urashima et al. 2008). To date, transcriptomic studies of human tissue have been either limited to HF patients with echocardiographic RV dysfunction but lacking RVF (di Salvo et al. 2015; di Salvo et al. 2015) or have been underpowered for statistical analysis (Williams et al. 2018). All these prior studies relied entirely upon the biased approach of pathway analyses of select differentially expressed genes. Although widely used, such pathway analyses are limited by an emphasis on individual genes, the resolution of the associated knowledge base, incorrect and inaccurate annotations, and lack of dynamic information (Khatri et al. 2012). None of the prior studies pursued experimental validation or mechanistic studies of their potential molecular signatures of RV dysfunction.

Accordingly, there is a need for the exploration of potential targets for RV-specific therapy in cardiopulmonary disease. This disclosure is directed to meet these and other needs.

### SUMMARY OF THE DISCLOSURE

In the present disclosure, the ventricular transcriptome of advanced HF patients, with versus without hemodynamically significant RVF (and thereby biventricular heart failure), was analyzed to identify gene networks that may be uniquely altered in RVF. Weighted gene co-expression network analysis (WGCNA) was integrated with detailed hemodynamic indices of advanced HF patients to identify a gene network (module) that correlated specifically with RVF. By validating gene hubs and drivers of this network in murine HF models, Wipi1 was identified as a conserved mediator of RVF. Furthermore, in isolated neonatal rat cardiac myocytes subjected to aldosterone, silencing Wipi1 partially prevented neurohormone-induced failing phenotype, restored physiological balance of non-canonical versus canonical autophagy, and blunted mitochondrial superoxide levels, suggesting Wipi1 as a potential target for therapeutic intervention.

One embodiment of the present disclosure is a method for treating or ameliorating the effect of a cardiopulmonary disease in a subject. This method comprises modulating the expression of at least one gene of a gene module associated with right ventricular failure (RVF) in the subject.

Another embodiment of the present disclosure is a method for diagnosing right ventricular failure (RVF) in a subject. This method comprises: (a) obtaining a biological sample from the subject; (b) determining the expression level of at least one gene of a gene module in the sample and comparing it to a reference determined in a healthy subject; (c) diagnosing the subject as being at risk for right ventricular failure (RVF) if the expression level of the at least one gene of the gene module in the sample is significantly higher than the reference; and (d) initiating a treatment protocol for the subject diagnosed in step (c) as being at risk for RVF.

Another embodiment of the present disclosure is a method for preventing right ventricular failure (RVF) in a subject. This method comprises decreasing the expression of WIPI1, in the subject.

Yet another embodiment of the present disclosure is a method for preventing non-canonical autophagy in a cardiac myocyte. This method comprises decreasing the expression of WIPI1, in the cardiac myocyte.

Still another embodiment of the present disclosure is a method for mitigating oxidative stress in mitochondria of a cardiac myocyte. This method comprises decreasing the expression of WIPI1, in the cardiac myocyte.

Another embodiment of the present disclosure is a method for differentially diagnosing right ventricular failure (RVF) from other diseases in a subject. This method comprises: (a) obtaining a biological sample from the subject; (b) determining the expression level of WIPI1 in the sample and comparing it to a reference determined in a healthy subject; (c) diagnosing the subject as being at risk for RVF if the expression level of WIPI1 in the sample is significantly higher compared to the reference; and (d) initiating a treatment protocol for the subject diagnosed in step (c) as being at risk for RVF.

A further embodiment of the present disclosure is a method for inhibiting microtubule-mediated active mRNA transfer in a cell. This method comprises decreasing the expression of at least one of WIPI1 and MAP4 in the cell. Preferably, the cell is a cardiac myocyte.

Another embodiment of the present disclosure is a pharmaceutical composition comprising: a first vector expressing CRISPR associated protein 9 (CAS9), a second vector expressing WIPI1 gRNA, and a pharmaceutically acceptable carrier.

Still another embodiment of the present disclosure is a method for treating or ameliorating the effect of a cardiopulmonary disease in a subject, comprising administering to the subject a therapeutically effective amount of a pharmaceutical composition disclosed herein.

### BRIEF DESCRIPTION OF THE DRAWINGS

The application file contains at least one drawing executed in color. Copies of this patent application with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 is a visualization of lightgreen module correlated with composite right ventricular failure (RVF) index. All members of the lightgreen module are connected at a co-expression correlation threshold of 0.667. The module network is filtered to show edges between transcript pairs with a co-expression correlation value >0.88. Gene set pathway analysis revealed a trilobar structure with signaling themes. Cyan edges delineate the “Cardiac Signaling Lobe”; purple edges delineate the “Innate Immunity Lobe”, and dark blue edges delineate the “Intracellular Signaling Lobe”. Node size reflects the betweeness centrality of the transcript; the larger the node size, the greater the betweeness centrality. Each node is also correlated to a composite index of hemodynamic parameters associated with RVF—right atrial pressure (RA), mean arterial pressure to right atrial pressure ratio (MAP:RA), and pulmonary artery systolic pressure (PASP). Positive correlations between transcript node and composite RVF index are represented in orange to red, thereby designating “drivers” of RVF. Negative correlations between transcript node and composite RVF index are represented in green, thereby designating “repressors” of RVF.

FIGS. 2A-2G show that pulmonary artery banding (PAB) induces progressive dilatation, functional decline, and eventual failure of the right ventricle (RV) over a 9-week course. C57BL/6J WT male mice were subjected to Sham or PAB surgery and assessed at 3-week intervals, up to 9 weeks post-surgery.

FIG. 2A shows the representative echocardiographic images of tissue doppler assessment of RV systolic function by lateral tricuspid annular systolic velocity S′.

FIG. 2B shows the representative echocardiographic images of M-mode assessment of TAPSE (tricuspid annulus planar systolic excursion).

FIG. 2C shows the representative echocardiographic images of B-mode assessment of RV FAC (fractional area change) and RV diastolic dimensions (a, major axis dimension; b, mid-cavity dimension; c, basal dimension).

FIG. 2D is the summary of echocardiographic assessment of RV function and structure, n=10-15 per group.

FIG. 2E shows terminal hemodynamic assessment of RV pressures (RVSP, RV peak systolic pressure; RVEDP, RV end-diastolic pressure) and stroke volume (SV) at 6- and 9-week post-surgery for PAB and Sham mice, n=5 per Sham group, n=4 per PAB group.

FIG. 2F shows morphometric assessment of hepatic congestion (Liver/TL, liver weight/tibia length), pulmonary edema (Lung/TL, lung weight/tibia length), and peripheral edema (BW/TL), n=7-17 per group.

FIG. 2G shows RT-qPCR analysis of fetal gene program in RV myocardium of PAB9wk versus Sham9wk, n=7-10 per group. *p<0.0001, †p<0.1, § p<0.01, ‡p<0.05, **p<0.001 vs respective Sham unless otherwise indicated by comparison bar, on Tukey's multiple comparison testing following two-way ANOVA for panels D-F; on two-tailed, unpaired Student's t-test for panel G. Scatter dot plots with bars show individual values and mean±SEM. Box-whisker plots show mean (+), median (midline), 25th and 75th percentiles (box), minimum, and maximum values.

FIGS. 3A-3C show transcript and protein expression of WGCNA identified, RVF-associated gene hub, drivers, and repressor in pulmonary artery banding (PAB) mouse model. RNA and protein were extracted from the RV of C57BL/6J WT male mice subjected to Sham or PAB. *p<0.001, †p<0.01, ‡p<0.05, § p=0.06 on Tukey's multiple comparison testing following two-way ANOVA for FIG. 3A; on two-tailed, unpaired Student's t-test for FIG. 3C. n=5-10 per group. Scatter dot plots with bars show individual values and mean±SEM.

FIG. 3A is RT-qPCR analysis of Wipi1, Hspb6, Snap47, Map4, and Prdx5 in RV of Sham and PAB mice at 3-, 6-, and 9-week post-surgery.

FIG. 3B shows the representative Western blots.

FIG. 3C is the summary densitometry analysis of Westerns, normalized to total protein stain, relative to Sham control. Total protein stain not shown.

FIGS. 4A-4C show the effect of silencing Wipi1 on hub, drivers, and repressor of WGCNA identified right ventricular failure (RVF)-associated module. Neonatal rat ventricular myocytes (NRVMs) were transfected with scramble or Wipi1-specific siRNAs and then stimulated with aldosterone (Aldo, 1 μM) for 48 h.

FIG. 4A shows the effect of Aldo stimulation and Wipi1 silencing on transcript levels of WGCNA identified RVF-associated genetic hub, drivers, and repressor (n=9 per group from 3 independent experiments).

FIG. 4B shows the representative Western blot.

FIG. 4C is the summary of Western analysis (n=9-12 per group from 3-4 independent experiments). *p<0.001, †p<0.0001, ‡p<0.01, § p<0.05 on Tukey's multiple comparison testing following two-way ANOVA. Scatter dot plots with bars show individual values and mean±SEM.

FIGS. 5A-5C show that non-canonical autophagy is upregulated in the failing right ventricle (RV) of pulmonary artery banding (PAB) mouse model. Protein lysates were prepared from RV of 9wk Sham or PAB operated C57BL/6J WT male mice.

FIG. 5A shows Western blots of autophagy proteins and total protein stain in non-failing Sham9wk-RV and failing PAB9wk-RV. The HSPB6 and WIPI1 blots shown are reused from FIG. 3B.

FIG. 5B shows that summary of Western analyses reveals upregulation of BECN1, HSPB6, WIPI1, and non-lipidated LC3 (LC3I) without an increase in LC3 lipidation (LC3II and LC3II/I ratio) in failing PAB9wk-RV versus non-failing Sham9wk-RV. This suggests a shift towards non-canonical autophagy pathways in the failing RV. *p<0.05, †p<0.001 on two-tailed Student's t-test; n=8 per group. Scatter dot plots with bars show individual values and mean±SEM.

FIG. 5C shows that summary of Western analyses reveals upregulation of BECN1, HSPB6, WIPI1, and non-lipidated LC3 (LC3I) without an increase in Ser16 phosphorylation of HSPB6 in failing PAB9wk-RV versus non-failing Sham9wk-RV. This suggests a shift towards non-canonical autophagy pathways in the failing RV. *p<0.05, †p<0.001 on two-tailed Student's t-test; n=8 per group. Scatter dot plots with bars show individual values and mean±SEM.

FIGS. 6A-6D show that silencing Wipi1 blunts aldosterone induction of non-canonical autophagy. Neonatal rat ventricular myocytes (NRVMs) were transfected with scramble or Wipi1-specific siRNAs and then stimulated with aldosterone (Aldo, 1 μM, 48 h), bafilomycin A (BafA, 100 nM, 1 h), or chloroquine (CQ, 100 μM, 1 h).

FIG. 6A shows the representative Western blots of autophagy proteins (n=9-18 per group from 3-6 independent experiments).

FIG. 6B shows summary Western analysis of LC3 lipidation (LC3II/LC3I) and canonical autophagy (pS16-/total HSPB6).

FIG. 6C shows representative Western blots of LC3 and WIPI1 in si-scramble versus si-Wipi transfected NRVMs treated with BafA or CQ to differentiate, respectively, canonical versus non-canonical autophagy. BafA blocks LCII lysosomal degradation during canonical autophagy, whereas CQ inhibits the fusion between the autophagosome and lysosome. Hence, CQ reveals total autophagic flux and the difference between the effects of CQ and BafA on LC3II/I ratios is attributable to non-canonical autophagy.

FIG. 6D is quantification of LC3II/I ratio which shows that silencing Wipi1 selectively inhibits non-canonical autophagy (CQ) without affecting canonical autophagy (BafA) (n=11 per group from 4 independent experiments for LC3II/I). *p<0.0001, ‡p<0.01, †p<0.05 versus basal, unless otherwise indicated by comparison bar, on Tukey's multiple comparison test following two-way ANOVA. Scatter dot plots with bars show individual values and mean±SEM.

FIGS. 7A-7C show that silencing Wipi1 decreases mitochondrial superoxide (O₂.) levels in in vitro neuro-hormonal model of right ventricular failure (RVF). Neonatal rat ventricular myocytes (NRVMs) were transfected with scramble or Wipi1-specific siRNAs and then stimulated with aldosterone (Aldo, 1 μM, 48 h) or hydrogen peroxide (H₂O₂, 50 μM, 2 h).

FIG. 7A shows brightfield and MitoSOX red imaging of NRVMs transfected with si-scramble versus si-Wipi1, with and without aldosterone (Aldo) stimulation. H₂O₂ was used as a positive control. (n=39-44 per group from 6 independent experiments). Scale bar measures 75 μM.

FIG. 7B shows summary analysis of mitochondrial O₂. levels (n=41-44 per group from 6 independent experiments).

FIG. 7C shows cell viability as assessed by MTT assay (n=24 per group from 3 independent experiments). *p<0.0001 versus respective unstimulated baseline unless indicated otherwise by comparison bar, on Tukey's multiple comparison test following two-way ANOVA. Box and whisker plots show mean (+), median (midline), 25th and 75th percentiles (box), minimum and maximum values (whiskers).

FIGS. 8A-8D show that silencing Wipi1 mitigates aldosterone induced oxidation of mitochondrial proteins CYPD and TRX2. Neonatal rat ventricular myocytes (NRVMs) were treated with oxidizing or reducing agents and then subjected to urea lysis, iodoacetamide-iodoacetic acid (IAM-IAA) alkylation, and Western analysis.

FIG. 8A is a schematic representation of the IAM-IAA alkylation method for identifying oxidized and reduced proteins in native non-reducing urea PAGE.

FIG. 8B shows representative redox Western blots for CYPD and TRX2 of NRVMs treated with: 1) Control, 2) reducing agent N-acetyl cysteine (NAC), 3) oxidizing agent hydrogen peroxide (H₂O₂), 4) aldosterone (Aldo). Black arrowhead, reduced protein band. Red arrow, oxidized protein band.

FIG. 8C is a histogram of Western densitometry analysis of CYPD and TRX2 oxidation.

FIG. 8D shows the quantification of CYPD and TRX2 oxidation under different redox conditions as shown by the ratio of oxidized to reduced protein band signals (n=3 per group). *p<0.05 on one-tailed Student's t-test; Scatter dot plots with bars show individual values and mean±SEM.

FIG. 9 is a proposed schematic of Wipi1 signaling mechanisms underlying right ventricular failure (RVF). RV pressure overload and chronic aldosterone activation upregulate WIPI1 signaling in the failing RV. Enhanced WIPI1 signaling increases mitochondrial superoxide levels and non-canonical autophagic flux. WIPI1 upregulation also correlates with increased Map4 expression, thereby potentially triggering MAP4-mediated myocyte contractile dysfunction or inhibition of microtubule-mediated active mRNA transfer. In vitro studies in neonatal rat ventricular myocytes demonstrated the feasibility of mitigating aldosterone-induced mitochondrial superoxide levels, blunting noncanonical autophagy, and decreasing Map4 expression by silencing Wipi1. Further studies are warranted to elucidate mechanistic details of WIPI1 signaling and to confirm the therapeutic potential of targeting WIPI1 in RVF.

FIGS. 10A-10C show weighted gene co-expression network analysis (WGCNA) gene dendrogram, modules, and module-phenotype correlation analysis.

FIG. 10A shows that Gene modules were identified using WGCNA dendrograms derived from the right ventricular transcriptome. The dynamic tree-cut algorithm was used to identify break points in the gene-tree, thereby indicating different clusters of related genes.

FIG. 10B is cytoscape visualization of the 23 RV-derived gene network modules identified. Color represents a distinct module. Line intensity and length indicate strength of individual interactions between gene pairs. Darker, shorter lines represent stronger connections than lighter, longer lines.

FIG. 10C shows that Module-phenotype relationship heatmap matrix for hemodynamic and echocardiographic indices was created to identify a module associated with right ventricular failure (RVF). Matrix cell color reflects Pearson's correlation value of module-to-phenotype. Correlation p-values are shown in cells. The lightgreen module was positively correlated with RA and RA:PCWP and negatively correlated with CI, independent of LVEDD and LVEF, thereby standing out as being associated with RVF. RA, right atrial pressure; PASP, pulmonary artery systolic pressure; PCWP, pulmonary capillary wedge pressure; SBP, systolic BP; DBP, diastolic BP; MAP, mean arterial pressure; CI, cardiac index; LVEDD, left ventricular end-diastolic diameter; LVEF, LV ejection fraction; TR, tricuspid regurgitation.

FIGS. 11A-11C show expression of WGCNA-identified RVF-associated gene hub, drivers, and repressor do not change in the failing left ventricle (LV). C57BL/6J WT male mice were subjected to Sham or transverse aortic constriction (TAC) and assessed at 3- and 6-week post-surgery. *p<0.0001, †p<0.01, § p<0.05, ‡p<0.001 versus respective Sham, unless otherwise indicated by comparison bar, on Tukey's multiple comparison test following two-way ANOVA for FIG. 11A, and on two-tailed, unpaired Student's t-test for FIGS. 11B and 11C. Scatter dot plots show individual values and mean±SEM.

FIG. 11A shows that serial echocardiograms and terminal morphometrics reveal changes in LV function (LVFS, LV fractional shortening), LV dilatation (LVEDD, LV end-diastolic diameter), LV hypertrophy (LV/TL, LV weight/tibia length ratio), and pulmonary edema (Lung/TL, lung weight/tibia length ratio) over time. n=7-18 per group.

FIG. 11B is RT-qPCR analysis of a fetal gene program. n=6-8 per group.

FIG. 11C is RT-qPCR analysis of WGCNA-identified RVF-associated gene hub, drivers, and repressor. n=6-8 per group.

FIG. 12 shows effect of silencing Wipi1 on aldosterone induction of fetal gene program in neonatal rat ventricular myocytes (NRVMs). NRVMs were transfected with scramble or Wipi1-specific siRNAs and stimulated with aldosterone (Aldo, 1 μM, 48 h). Fetal gene program is induced by Aldo stimulation. Silencing Wipi1 blunts aldosterone-induced upregulation of Myh7 (n=9 per group from 3 independent experiments). *p<0.001, †p<0.0001, and ‡p<0.01 on Tukey's multiple comparison testing following two-way ANOVA. Scatter plots with bars show individual values and mean±SEM.

FIG. 13 is heat map of autophagy genes from human ventricular transcriptomic analysis. Color key shows differential expression (log 2(fold change)) relative to respective non-failing ventricle. RV, right ventricle; LV, left ventricle; LV-HF, LV failure without hemodynamically significant RV failure; BiV-HF, biventricular failure with hemodynamically significant RV failure. Purple arrows, genes that were differentially expressed in the failing RV (BiV-HF RV) and that distinguish the failing RV from the dysfunctional RV (LV-HF RV). Red arrows, genes that were differentially expressed in the failing RV (BiV-HF RV) and that distinguish the failing RV from the failing LV (BiV-HF LV). n=5 per group, per ventricle. Group means are represented in the heat map.

FIGS. 14A-14C show upregulation of canonical autophagy in transverse aortic constriction (TAC)-induced left ventricular failure. Protein lysates were prepared from the left ventricle (LV) of adult C57BL/6J WT male mice subjected to Sham or TAC for 6 weeks.

FIG. 14A shows Western blots of autophagy proteins and total protein stain.

FIG. 14B shows the summary Western analyses. Upregulation of select autophagy proteins in the absence of increased LC3 lipidation in TAC6wk-LV versus Sham6wk-LV suggests that overall autophagic flux is unchanged in the failing versus non-failing LV.

FIG. 14C shows that increased Ser16-phosphorylation of HSPB6 in TAC6wk-LV suggests a shift towards increased canonical autophagy in the failing LV. *p<0.05, †p<0.001 on two-tailed Student's t-test; n=6 per group. Scatter dot plots show individual values and mean±SEM.

FIG. 15 is principal component analysis plot of a right ventricular failure-associated module. The first principal component accounts for the vast majority (76.4%) of the information in the module.

FIG. 16 is a schematic of representative constructs of AAV9 vectors for RV-specific deletion of WIPI1 useful for treating cardiopulmonary disease such as, e.g., RVF, in a human.

### DETAILED DESCRIPTION OF THE DISCLOSURE

One embodiment of the present disclosure is a method for treating or ameliorating the effect of a cardiopulmonary disease in a subject. This method comprises modulating the expression of at least one gene of a gene module associated with right ventricular failure (RVF) in the subject.

In some embodiments, the gene module comprises the following genes: WIPI1, HSPB6, MAP4, SNAP47, and PRDX.

In some embodiments, the modulation comprises decreasing the expression of at least one of WIPI1, HSPB6, MAP4, and SNAP47, and/or increasing the expression of PRDX, in the subject. In some embodiments, the modulation comprises decreasing the expression of WIPI1, HSPB6, and MAP4, in the subject. In some embodiments, the modulation comprises decreasing the expression of WIPI1, in the subject.

In some embodiments, the cardiopulmonary disease is associated with right ventricular failure (RVF). As used herein, a “cardiopulmonary disease” refers to a diverse group of serious disorders affecting the heart and lungs. Non-limiting examples of a cardiopulmonary disease include hypertension, stroke and coronary heart disease. In some embodiments, the cardiopulmonary disease is selected from heart failure and pulmonary hypertension.

In some embodiments, the subject is a mammal, which can be selected from the group consisting of humans, primates, farm animals, and domestic animals. Preferably, the mammal is a human.

Another embodiment of the present disclosure is a method for diagnosing right ventricular failure (RVF) in a subject. This method comprises: (a) obtaining a biological sample from the subject; (b) determining the expression level of at least one gene of a gene module in the sample and comparing it to a reference determined in a healthy subject; (c) diagnosing the subject as being at risk for right ventricular failure (RVF) if the expression level of the at least one gene of the gene module in the sample is significantly higher than the reference; and (d) initiating a treatment protocol for the subject diagnosed in step (c) as being at risk for RVF.

In some embodiments, the gene module comprises the following genes: WIPI1, HSPB6, MAP4. In some embodiments, the at least one gene is WIPI1. In some embodiments, the treatment protocol comprises modulating WIPI1 expression.

As used herein, a “biological sample” includes any appropriate material obtained from the subject and may include one or more of blood, serum, plasma, urine, body tissue or other body fluid. Generally, a biological sample is a sample containing serum, blood or plasma. To obtain the biological sample from the subject, conventional methods such as blood draws and biopsies may be used as determined appropriate by a medical professional.

Another embodiment of the present disclosure is a method for preventing right ventricular failure (RVF) in a subject. This method comprises decreasing the expression of WIPI1, in the subject.

In some embodiments, the subject has at least one of the following: right ventricular dysfunction (RVD), reduced ejection fraction, preserved ejection fraction, a left ventricular assist device, pulmonary hypertension, and cardiovascular etiology.

Yet another embodiment of the present disclosure is a method for preventing non-canonical autophagy in a cardiac myocyte. This method comprises decreasing the expression of WIPI1, in the cardiac myocyte.

In some embodiments, the non-canonical autophagy is induced by a neurohormone. As used herein, a “neurohormone” is any hormone produced and released by neuroendocrine cells (also called neurosecretory cells) into the blood. In some embodiments, the neurohormone is aldosterone.

Still another embodiment of the present disclosure is a method for mitigating oxidative stress in mitochondria of a cardiac myocyte. This method comprises decreasing the expression of WIPI1, in the cardiac myocyte.

In some embodiments, the oxidative stress is aldosterone-induced. In some embodiments, the oxidative stress is not induced by hydrogen peroxide.

Another embodiment of the present disclosure is a method for differentially diagnosing right ventricular failure (RVF) from other diseases in a subject. This method comprises: (a) obtaining a biological sample from the subject; (b) determining the expression level of WIPI1 in the sample and comparing it to a reference determined in a healthy subject; (c) diagnosing the subject as being at risk for RVF if the expression level of WIPI1 in the sample is significantly higher compared to the reference; and (d) initiating a treatment protocol for the subject diagnosed in step (c) as being at risk for RVF.

In some embodiments, the other diseases include right ventricular dysfunction, progressive right ventricular dilatation, and left ventricular failure (LVF).

A further embodiment of the present disclosure is a method for inhibiting microtubule-mediated active mRNA transfer in a cell. This method comprises decreasing the expression of at least one of WIPI1 and MAP4 in the cell. Preferably, the cell is a cardiac myocyte.

Another embodiment of the present disclosure is a pharmaceutical composition comprising: a first vector expressing CRISPR associated protein 9 (CAS9), a second vector expressing WIPI1 gRNA, and a pharmaceutically acceptable carrier.

In some embodiments, the vector is a viral vector selected from the group consisting of adenovirus, adeno-associated virus (AAV), alphavirus, vaccinia virus, lentivirus, herpes virus, and retrovirus. In some embodiments, the adeno-associated virus (AAV) is selected from the group consisting of AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), and AAV serotype 11 (AAV11). In some embodiments, the vector is an AAV9 viral vector.

In some embodiments, the first vector contains an inducible sequence, a cell-specific promoter rejoin, and a sequence encoding CAS9. In some embodiments, the first vector provides for inducible, cardiac myocyte specific expression of CAS9. In some embodiments, the inducible sequence comprises a tetracycline response element (TRE). In some embodiments, the cell-specific promoter region comprises a cardiac troponin T (TNNT2) promotor.

In some embodiments, the second vector contains a cytochrome P450 (CYP450) promoter region and a sequence encoding WIPI1 gRNA. In some embodiments, the second vector provides for RV-specific expression of a gene module of the present disclosure, such as human WIPI1. Non-limiting examples of CYP450 promoter regions include CYP3A4/5, CYP2D6, CYP2C8/9, CYP1A2, CYP2C19, CYP2E1, CYP2B6, and CYP2A6. In some embodiments, the CYP450 promoter is CYP2D6. In some embodiments, the WIPI1 gRNA is human WIPI1 gRNA. In some embodiments, the functional cassettes of the first and second vectors are present in a single vector, e.g., a single AAV9 vector. The vector or vectors, as the case may be, may be delivered directly to a subject or may be combined in a pharmaceutically acceptable composition for delivery to the subject.

Still another embodiment of the present disclosure is a method for treating or ameliorating the effect of a cardiopulmonary disease in a subject, comprising administering to the subject a therapeutically effective amount of a pharmaceutical composition disclosed herein.

As used herein, the term “administering” means oral administration, administration as a suppository, topical contact, intravenous, intraperitoneal, intramuscular, intralesional, intranasal or subcutaneous administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, intracoronary and intracranial. Other modes of delivery include, but are not limited to, the use of liposomal formulations, intravenous infusion, transdermal patches, microbubbles (including ultrasound-mediated microbubble destruction), and the like. In some embodiments, the pharmaceutical composition disclosed herein is administered via intracoronary injection to the right coronary artery (RCA). In some embodiments, the compositions of the invention, such as the first and second vectors disclosed herein, are administered using any procedure that specifically delivers the composition to the target tissue, e.g., the right ventricle of a human patient.

In the present disclosure, an “effective amount” or “therapeutically effective amount” of a vector or pharmaceutical composition is an amount of such a vector or composition that is sufficient to affect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a vector or pharmaceutical composition according to the disclosure will be that amount of the vector or composition, which is the lowest dose effective to produce the desired effect with no or minimal side effects. The effective dose of a vector or pharmaceutical composition according to the present disclosure may be administered as two, three, four, five, six or more sub-doses, administered separately at appropriate intervals throughout the day.

The disclosure is further illustrated by the following examples, which are offered for illustrative purposes, and are not intended to limit the disclosure in any manner. Those of skill in the art will readily recognize a variety of noncritical parameters, which can be changed or modified to yield essentially the same results.

### EXAMPLES

### Example 1

### Methods and Materials

### Study Design

The goal of this study was to identify unique genetic determinants of RV failure that might be targeted for the development of novel RVF-specific therapy. Towards this end, we leveraged transcriptomic data from human ventricular tissue of advanced heart failure patients with versus without hemodynamically significant RV failure (and thereby biventricular HF) as well as that from non-failing donors (n=5 patients per group). Using WGCNA, module-trait analysis, and subsequent gene-phenotype correlations, we identified genes likely to mediate RVF. We experimentally validated some of the candidate RVF-associated genes in mouse models of pressure-overload induced RV versus LV failure. We subsequently focused our attention on the genetic hub Wipi1 which was upregulated in the failing RV of human patients and mouse models and also correlated with other identified RVF-associated genetic drivers. To elucidate possible pathophysiological mechanism of Wipi1 and test its potential as a therapeutic target, we performed in vitro isolated cardiac myocyte cell culture studies, in which cells were subjected to neurohormonal activation associated with RVF, namely chronic aldosterone activation. We silenced Wipi1 in this in vitro cell culture model and assessed the effect on autophagy, mitochondrial superoxide levels, and the fetal gene program associated with heart failure.

### Human Ventricular Tissue Samples

Human ventricular myocardium was obtained from end-stage ischemic cardiomyopathic hearts explanted at the time of cardiac transplantation. Non-failing donor hearts that had been deemed unsuitable for transplantation were used as control. Prior to explant, hearts underwent intra-operative antegrade coronary perfusion with 4:1 blood cardioplegia solution. Following arrest, hearts were explanted and placed into cold Ca²⁺-free, modified Krebs-Henseleit solution as previously described (Dipla et al. 1998). Samples were taken from mid-myocardial regions of the LV free wall and the RV free wall, in areas void of scar tissue. Tissue samples were rapidly frozen in liquid nitrogen and stored at −80° C. until RNA isolation. Prospective written informed consent for research use of heart tissue was obtained from all transplant recipients or next of kin (non-failing donors). Patient consent, sample collection and preparation, and clinical data collection were performed according to a human subject research protocol approved by the Institutional Review Board of the Lewis Katz School of Medicine at Temple University.

### RNAseq

End-stage ischemic cardiomyopathic hearts were selected for RNA sequencing based upon patient's invasive hemodynamic parameters prior to transplantation and the absence of left ventricular assist device as a bridge to transplantation. The LV-HF cohort, defined as those without hemodynamic evidence of RVD or RVF, were selected based upon RA<8 mmHg and RA:PCWP<0.5. The BiV-HF cohort, defined as those with hemodynamic evidence of RVD and RVF and thereby biventricular HF, were selected based upon RA>15 mmHg, and RA:PCWP>0.62. These hemodynamic criteria for RVF were based upon prior studies establishing cutoff values for RA (Atluri et al. 2013) and RA:PCWP (Drazner et al. 2013; Kormos et al. 2010) in advanced heart failure patients. Only matched LV and RV tissue (from the same patient) were used.

RNA extraction was performed with a Total RNA Purification Plus Micro Kit (Norgen Biotek), according to the manufacturer's instructions. RNA sequencing was performed by LC Sciences (Houston, Tex.) with the Illumina platform.

### WGCNA

Networks were generated as previously described (Langfelder et al. 2008) using the parameters provided on-line (http://labs.genetics.ucla.edu/horvath/htdocs/CoexpressionNetwork/Rpackages/WGCNA/Tutorials/). Briefly, Pearson correlations were determined for each pair of expressed (average FPKM>1) and varying (coefficient of variation>10%) transcripts. Correlations were transformed to approximate a scale-free degree distribution by raising each correlation to the power of 6 as recommended by pickSoftThreshold algorithm within WGCNA. Topological Overlap (TOM) was calculated as follows:

$${TOM}_{i j} = \frac{{Σ}_{u} \{ {A}_{i u} {A}_{u j} \} + {A}_{i j}}{min ( {k}_{i} , {k}_{j} ) + 1 - {A}_{i j}}$$

where i, j are a pair of transcripts, u is the set of all other transcripts, A is the adjusted correlation matrix, and k is the degree of the node. Modules were identified using the dynamic tree cut algorithm on the DistTOM (1-TOM) matrix and eigengenes were determined from the first principle component of the genes in each module. Modules whose eigengenes have a Pearson correlation of greater than 0.8 were merged.

The WGCNA method was implemented in the freely available WGCNA R package (Langfelder et al. 2008).

### Module Selection and Enrichment

Eigengenes of the RV-only modules were correlated to RVF hemodynamic indices, and modules with significant correlations (p<0.05) were further filtered to determine which RV modules were not preserved or weakly preserved in the combined network. This RV-specific, RVF-associated module was subsequently used to identify hubs and drivers. GeneAnalytics was used to identify enriched biological categories in the genes of our modules of interest (Ben-Ari et al. 2016). Significance was determined by a Bejamini-Hochberg corrected binomial test p<0.05.

### Hubs

Betweenness centrality was calculated for each transcript using the NetworkAnalyzer tool in Cytoscape (Assenov et al. 2008). For a given node n in module G, the normalized betweenness centrality C_{b}(n) is:

$${C}_{b} ( n ) = \frac{2 {Σ}_{s \neq n \neq t} ( {σ}_{st} ( n ) / {σ}_{st} )}{( N - 1 ) ( N - 2 )}$$

where s and t are nodes in G different from n, σₛₜ is the number of shortest paths from s to t and σₛₜ(n) represents the number of shortest paths from s to t which pass through n. N is the total number of nodes. Transcripts with significant betweenness centralities (‘Hubs’) have increased importance to overall modular structure. Significance was calculated by bootstrapping 100,000 networks with the same number of nodes and preserved degree structure using the degree.sequence.game function from the R package igraph (Csardi and Nepusz, 2006) and an overall significance threshold (0.00029) determined by Bonferroni correction.

### Drivers and Repressors

Drivers and repressors are genes connected to the rest of the module which respectively show strong independent positive or negative correlation with RVF hemodynamic indices. Genes with low betweenness centralities (lowest quartile) were removed. Genes significantly correlated to RVF hemodynamic indices were ranked based on p-value. Potential candidate drivers and repressors were selected based upon significant correlations, betweenness centrality, and previously validated expression in human cardiac tissue (Fagerberg et al. 2014).

### Module Visualization

Module visualization was performed using Cytoscape 3.4 (Shannon et al. 2003). Node size reflected that node's betweenness centrality; node color reflected the transcript's correlation (direction and strength) to a composite RVF phenotype index averaging the correlation values of each transcript with RAP, PASP, and MAP:RA. The negative of the MAP:RA correlation value was used in this averaged index since MAP:RA is inversely related to RVF. Hence, drivers have positive correlations to the RVF phenotype index, and repressors will have negative correlations. Green node color indicates at least modest negative (R²<−0.4), yellow indicates minimal (−0.4≤R²≤0.4), and red indicating at least modest positive (R²>0.4) phenotypic correlations. Module layout was determined via the “edge-weighted spring embedded” layout algorithm using the correlation strength between individual gene expressions as the edge weights. Edges with R<0.88 were removed to aid visualization.

### Heatmaps

Heatmaps were generated using the heatmap.2 function from the R package gplots (Warnes et al. 2016).

### RNA Isolation and Quantitative RT-PCR

Total RNA were extracted from human and mouse tissues and cultured neonatal rat ventricular myocytes (NRVMs) using the Tissue RNA Purification Kit (Norgen Biotek) and the TRIzol reagent (Invitrogen) respectively, according to the manufacturer's instructions. RNA was quantified with the NanoDrop-2000c instrument (Thermo Scientific), and cDNA synthesis was performed with iScript reverse transcription Supermix (BioRad). Quantitative RT-PCR was performed on a CFX96 thermal cycler using the iTac Universal SYBR green Supermix (Biorad) and specific primers. Gene expression was normalized to Rps13, and relative mRNA expression was quantified using the ΔΔCt method. For data robustness and reproducibility, target genes were also normalized to Rps15. All primer sequences are listed in Table 1.

TABLE 1
Complete list of primers.

| Species | Gene | Primer | Sequence (5′-3′) |
|---|---|---|---|
| Mouse | Wipi1 | Forward | CTTTCAACCAAGACTGCACATC (SEQ ID NO: 1) |
| Rat | | Reverse | GTTCATCTGCCGAGGTTTTG (SEQ ID NO: 2) |
| Mouse | Hsbp6 | Forward | GCTCCTTTACCAGGTTTCTCTG (SEQ ID NO: 3) |
| | | Reverse | ATCCAGCAGCACGGAAAAATAC (SEQ ID NO: 4) |
| Rat | Hsbp6 | Forward | TTTACCGGGTTTTTCCACTCCG (SEQ ID NO: 5) |
| | | Reverse | CTTCACATCCAGCAGCACAGA (SEQ ID NO: 6) |
| Mouse | Map4 | Forward | CCCCAAAGAAACAGAGACAAC (SEQ ID NO: 7) |
| | | Reverse | CTGAGAGTGAAACCATGCC (SEQ ID NO: 8) |
| Rat | Map4 | Forward | CTCCTCTCTGCCCTCTCCC (SEQ ID NO: 9) |
| | | Reverse | CCGCCATTCTTTACCACTGC (SEQ ID NO: 10) |
| Mouse | Snap47 | Forward | GGAGCTGACACAGATCCTGA (SEQ ID NO: 11) |
| | | Reverse | CATACGCCGGTTTTGCTTGT (SEQ ID NO: 12) |
| Rat | Snap47 | Forward | CTTCTGCGCGCTCCTGTTG (SEQ ID NO: 13) |
| | | Reverse | AGGCCAGGTGTGAACTCGTA (SEQ ID NO: 14) |
| Mouse | Prdx5 | Forward | TGGCCTGTCTGAGCGTTAAT (SEQ ID NO: 15) |
| | | Reverse | GAGAACCTTTTCAGCCGACG (SEQ ID NO: 16) |
| Rat | Prdx5 | Forward | ACTATGGCCCCGATCAAG (SEQ ID NO: 17) |
| | | Reverse | GGAACAGCCAGGTGTAAATG (SEQ ID NO: 18) |
| Mouse | Myh6 | Forward | GTGACAGTGGTAAAGGCAAAGG (SEQ ID NO: 19) |
| Rat | | Reverse | TCAGATTTTCCCGGTGGAGA (SEQ ID NO: 20) |
| Mouse | Myh7 | Forward | GCAGCAAGAAGGACCAGACC (SEQ ID NO: 21) |
| Rat | | Reverse | TTTCCCAAATCGAGAGGAGTTG (SEQ ID NO: 22) |
| Mouse | Nppa | Forward | GCTTCGGGGGTAGGATTGAC (SEQ ID NO: 23) |
| Rat | | Reverse | TAGATGAAGGCAGGAAGCCG (SEQ ID NO: 24) |
| Mouse | Nppb | Forward | CTTCGGTCTCAAGGCAGCAC (SEQ ID NO: 25) |
| Rat | | Reverse | GAGACCCAGGCAGAGTCAGAA (SEQ ID NO: 26) |
| Mouse | Acta1 | Forward | AGCCTCACTTCCTACCCTCG (SEQ ID NO: 27) |
| Rat | | Reverse | TTGTCACACACAAGAGCGGT (SEQ ID NO: 28) |
| Mouse | Rps13 | Forward | GCACCTTGAGAGGAACAGAA (SEQ ID NO: 29) |
| Rat | | Reverse | GAGCACCCGCTTAGTCTTATAG (SEQ ID NO: 30) |
| Mouse | Rps15 | Forward | TTCTCCATCACCTACAAACCC (SEQ ID NO: 31) |
| | | Reverse | ACCAGTCTTTATTGGCCTCG (SEQ ID NO: 32) |
| Rat | Rps15 | Forward | GTTCTCCATCACCTACAAGCC (SEQ ID NO: 33) |
| | | Reverse | ACGAGTCTTTATTGTCCCCAC (SEQ ID NO: 34) |

### Protein Isolation and Western Blot Analysis

Total proteins were extracted from tissues and NRVMs with the T-PER lysis buffer (Thermo Scientific) supplemented with the Halt Protease and Phosphatase Inhibitor Cocktail (Thermo Scientific). Protein extraction from mouse tissues was performed using a BeadBug Benchtop Homogenizer and zirconium beads (Sigma). Protein quantification was performed with the BCA reagent (Pierce). Total protein samples were resolved on NuPAGE 4-12% Bis-Tris gradient gels (Invitrogen) and transferred to 0.2 μm Nitrocellulose membranes (Biorad) for immunoblotting. Membranes were stained with the LI-COR Revert Total Protein Stain kit and analyzed as per manufacturer's instructions. Membranes were blocked and then incubated with primary antibodies as detailed in Table 2 at 4° C. overnight. Membranes were then washed and incubated with the appropriate LI-COR secondary antibody. Membranes were imaged using the ODYSSEY-Classic infrared system from LI-COR. Protein expression was normalized to total protein. All antibodies are listed in Table 2.

TABLE 2
Complete list of antibodies.

| | Dilution | |
|---|---|---|
| | in WB | Company (Catalog no.) |
| Primary Antibodies | | |
| Anti-WIPI1 | 1:500 | Novus Biologicals (NBP1-88878) |
| Anti-HSPB6 | 1:2,000 | Fitzgerald (10R-H111A) |
| Anti-phospho Serine 16 | 1:1,000 | Fitzgerald (70R-36849) |
| HSPB6 | | |
| Anti-MAP4 | 1:5,000 | Millipore (AB6020) |
| Anti-SNAP47 | 1:500 | Aviva (ARP58429_P050) |
| Anti-PRDX5 | 1:500 | Aviva (ARP54832_P050) |
| Anti-BECN1 | 1:1,000 | Cell signalling (#3738) |
| Anti-LC3 (I and II) | 1:1,000 | Bio-Rad (AHP2167) |
| Anti-CyclophilinD | 1:1,000 | Thermo-Fisher (45-590-0) |
| (clone: E11AE12BD4) | | |
| Anti-Thioredoxin2 | 1:1,000 | Santa Cruz (F-10, sc-133201) |
| Secondary Antibodies | | |
| IRDye ® 800CW Goat anti- | 1:10,000 | LI-COR Biosciences (926-32211) |
| Rabbit IgG | | |
| IRDye ® 680RD Goat anti- | 1:10,000 | LI-COR Biosciences (926-68070) |
| Mouse IgG | | |
| IRDye ® 800CW Goat anti- | 1:10,000 | LI-COR Biosciences (926-32350) |
| Mouse IgG1 Specific | | |
| IRDye ® 800CW Goat anti- | 1:10,000 | LI-COR Biosciences (926-32352) |
| Mouse IgG2b Specific | | |

### Animal Experiments

Animal experiments were conducted in strict accordance with the recommendations in the Guide for the Care and Use of Laboratory Animal of the National Institutes of Health. The protocol for all animal procedures was approved by the Institutional Animal Care and Use Committee of Columbia University. Adult male C57BL/6 WT mice were purchased from Jackson Laboratory and subjected to Pulmonary artery banding (PAB), transverse aortic constriction (TAC), or Sham surgeries at age 10-12 wks (Tarnayski et al. 2004; Tsai et al. 2012). Animals were given pre-operative analgesia with meloxicam SR 4 mg/kg s.c. one day prior to surgery. Animals were anesthetized to surgical plane with ketamine/xylazine (80-100/5-10 mg/kg, i.p.), endotracheally intubated, and mechanically ventilated (MiniVent 845 Mouse Ventilator, Harvard Apparatus). Animals were subjected to PAB, TAC, or Sham surgery as detailed below, after which the thoracic cavity was closed in layers with a 6-0 nylon suture and the skin with 4-0 nylon sutures. After surgery, mice were gradually weaned from the ventilator until spontaneous respiration was resumed, and animals were then replaced in a cage to fully recover from anesthesia. Skin sutures were removed after two weeks.

### PAB Model

Pulmonary artery banding (PAB) was used to induce right ventricular pressure overload and eventual right ventricular failure in mice, as previously described (Tarnayski et al. 2004). After left thoracotomy, the pulmonary artery was carefully dissected free from the aorta and a 7-0 silk suture was gently tied around the proximal main PA, against a blunt 25g needle to yield a narrowing 0.5 mm in diameter when the needle was removed.

### TAC Model

Transverse aortic constriction (TAC) was used to induced left ventricular pressure overload and eventual left ventricular failure in mice, as previously described (Tarnayski et al. 2004; Tsai et al. 2012). Following thoracotomy, a 7-0 silk suture was tied around the transverse aorta between the takeoff of the innominate artery and that of the common carotid artery, against a blunt 27g needle to yield a narrowing 0.4 mm in diameter upon removal of the needle.

### Sham Model

For age-matched normal controls, mice underwent thoracotomy without tying a suture around either the PA or transverse aorta.

### Echocardiography

Mice were anesthetized with 1-2% inhalational isoflurane and transthoracic echocardiography was performed using a 18-38 MHz linear-array transducer probe with a digital ultrasound system (Vevo 2100 Image System, VisualSonics, Toronto, Canada), at 3, 6 and 9 wks after surgery. Vevo LAB 3.0 ultrasound analysis software (Fujifilm, VisualSonics) was used to measure and analyze the image data. Pulmonary artery and aortic pressure gradients were measured by pulse wave Doppler to confirm pulmonary artery banding or transverse aortic constriction. For assessment of LV structure and function, M-mode images were acquired in the parasternal short axis view to obtain: left ventricular end systolic and diastolic diameters (LVESD, LVEDD); LV fractional shortening (FS); LV posterior wall thickness; and LV anterior wall thickness. B-mode images acquired in the parasternal short axis view were used to obtain LV fractional area change (FAC). For assessment of RV structure and function (Kohut et al. 2016), M-mode images were acquired in the apical 4-chamber view to obtain tricuspid annular planar systolic excursion (TAPSE). B-mode images were acquired in the apical 4-chamber view to obtain: diastolic right ventricular dimensions from base to apex (RVD, major), at mid-cavity (RVD,mid), and at the base or tricuspid annulus (RVD,base); and RV fractional area change (RV FAC). The lateral tricuspid annular systolic velocity (RV S′) was acquired using Doppler tissue imaging in the apical 4-chamber view.

### Morphometric Analysis

At experimental endpoint at 3, 6, or 9 wks following surgery, mice were euthanized and heparinized, were collected in 1×PBS (Corning-LDP) containing penicillin (100 units/ml)/streptomycin (100 μg/ml) (Gibco-Fisher Scientific). Atria and great vessels were carefully dissected away and the remaining ventricular tissue was further minced using sterile razor blades and placed in 50 ml sterile tubes (24-36 hearts per tube). Subsequently, and ventricles, lungs and liver were removed and weighed. Hearts were carefully dissected into atria, right ventricular free wall (RVFW), interventricular septum (IVS), and left ventricular free wall (LVFW). Tissue weights were normalized to tibia length (TL) as appropriate to assess pulmonary edema (lung weight/TL), hepatic congestion (liver weight/TL), and LV hypertrophy (LV/TL).

### Isolation and Primary Culture of Neonatal Rat Ventricular Myocytes

Neonatal Sprague-Dawley rats were euthanized by decapitation within the first 24 h after birth and beating hearts heart fragments were rinsed in 1×PBS without pen/strep and digested in 0.1% Trypsin solution in 1×PBS (0.8 ml of 0.1% Trypsin solution per heart) for 15 min at 37° C. The supernatant was collected and the remaining tissue was further digested repeatedly for a total of 10 times, with serial collection of supernatant. Digestion was stopped on ice with 10% FBS, and cells were collected from pooled supernatant by centrifugation at 1500 rpm for 5 min at room temperature. The cell pellet was resuspended in an adequate volume of complete medium. Cells were counted and plated in 10 cm dishes for ˜1 h at 37° C., 5% CO₂ at a density of 10×10⁶ cells per plate (pre-plating). During this pre-plating step, non-myocytes including fibroblasts adhere to the plate, while NRVMs remain in suspension. The supernatant cells were subsequently seeded on protamine sulfate coated dishes (10⁵ cells/cm²) and were left to attach for 12 h. NRVM primary cultures were maintained in MEM medium supplemented with 10% FBS, penicillin (100 units/ml)/streptomycin (100 μg/ml) (Gibco-Fisher Scientific). 1-β-D-Arabinofuranosyl-cytosine (AraC 20 μM, Calbiochem-Sigma) was also added to the culture medium to inhibit fibroblast proliferation.

siRNA Transfection of NRVMs and In Vitro Model of Neurohormone Activation Associated with RVF

At 24 h following isolation, NRVMs were transfected with siRNAs using the Dharmafect #1 transfection reagent according to the manufacturers protocol (Dharmacon). NRVMs were plated at a density of ˜10⁵ cells/cm² and transfected with 10 nM of a pool of either non-targeting siRNA (siRNA-scramble control) or siRNAs against the rat Wipi1 gene (siRNA-Wipi1). All siRNAs were ON-TARGETplus SMARTpool siRNAs (Dharmacon). Following a 24 h transfection period, the medium was changed to serum-free DMEM:F12 supplemented with penicillin (100 units/ml)/streptomycin (100 μg/ml). After ˜12 h of serum-starvation, the cells were incubated with aldosterone (250 μg/ml) in serum-free DMEM:F12 culture medium for 48 h at 37° C., 5% Ca. Serum-free DMEM:F12 culture medium without any aldosterone was used as a comparative control to neurohormone activation.

### MTT Assay of Cell Viability

To assess NRVM viability, the Vybrant MTT cell proliferation assay kit was used (Molecular probes). Initially, NRVM were seeded on 96-well plates at ˜3×10⁴ cells/well (˜10⁵ cells/cm², the same density as in other assays) and were treated identically as in the siRNA transfection assays (including transfection, serum starvation and neurohormonal stimulation). In addition, increasing number of cells ranging from 2×10⁴ to 14×10⁴ were used to create a standard curve and to calculate the linearity between absorbance at 595 nm and cell number. Prior to labelling with MTT, the medium was removed and 100 μl of fresh medium was added to each well. The same amount of medium (100 μl) without cells was used as negative control (blank). The cells and the negative control were labeled with 10 μl of 12 mM MTT/well and were incubated at 37° C. for 4 h. Subsequently, 100 μl of SDS-HCL solution was added to each well, mixed thoroughly and incubated for 4 h at 37° C. Finally, the absorbance at 595 nm was measured on a plate reader and the % of cell viability was calculated using the formula:

% of viability=(A595sample/A595reference)×100

### MitoSOX Red Analysis

Mitochondrial superoxide level was monitored with the MitoSOX™ Red mitochondrial superoxide indicator for live-cell imaging (Molecular Probes). Briefly cells were incubated with 2.5 μM of MitoSOX red indicator in serum free culture medium for 20 min at 37° C. protected from light. Subsequently cells were washed with warm medium and were imaged on a DMI8 fluorescent microscope (Leica) using a red fluorescent filter with excitation/emission of approximately 510/580 nm. Cells incubated with H₂O₂ (50 μM) for 2 h were used as positive control for MitoSOX red staining. The red fluorescent signal was measured with ImageJ software and normalized to the brightfield signal.

### Statistical Analyses

All statistical analyses of in vivo mouse model and in vitro cell culture experiments were carried out using GraphPad Prism 7.0c. Unless otherwise specified, data are expressed as means±SEM. Mean comparisons between two groups were compared using unpaired Student's t-test or Mann-Whitney test, as appropriate. For multiple comparisons, one- or two-way ANOVAs were performed, followed by Tukey's or uncorrected Dunn's multiple comparison tests, as appropriate. Statistical significance was defined for two-tailed p<0.05. For the assessment of RNA-seq data of each of the candidate WGCNA-identified RVF-associated genetic hub, drivers, and repressor, statistical significance was defined as p<0.10, given the limited sample size, non-normal data distribution, and the use of human tissue analysis as a discovery rather than validation approach.

### Human Subjects Consent

Patient consent, sample collection and preparation, and clinical data collection were performed according to a human subject research protocol approved by the IRB of the Lewis Katz School of Medicine at Temple University.

### Example 2

### Clinical and Hemodynamic Characteristics of Advanced Heart Failure Patients

Clinical characteristics of advanced HFrEF patients without RVF and thereby with LV failure alone (LV-HF), advanced HFrEF patients with RVF and thereby biventricular failure (BiV-HF), and non-failing (NF) adult patients are listed in Table 3. The median (interquartile range) age was 61.5 (60.0-63.5) yrs for all advanced HF patients and 51.0 (43.0-52.0) yrs for non-failing donors. There was no significant difference in age between LV-HF and BiV-HF patients. As would be expected, BiV-HF patients had higher rates of inotropic medication use, lower rates of β-blocker use, lower LVEF, and worse hemodynamic indices of RV function than LV-HF patients (Tables 3 and 4). Specifically, BiV-HF patients had markedly elevated right atrial pressure (RAP), increased ratio of right atrial pressure to pulmonary capillary wedge pressure (RA:PCWP), lower systolic and mean arterial blood pressure (SBP and MAP, respectively), markedly decreased ratio of mean arterial pressure to right atrial pressure (MAP:RA), and lower cardiac index (CI) in spite of greater inotropic support.

TABLE 3
Clinical characteristics of non-failing patients and heart failure patients with and
without biventricular failure. NF, non-failing; LV-HF, left ventricular heart failure; BiV-HF,
biventricular heart failure; CAD, coronary artery disease; h/o, history of; CABG, coronary
artery bypass graft surgery; ACEi, angiotensin converting enzyme inhibitor; ARB,
angiotensin receptor blocker; MRA, minerolacorticoid receptor antagonist; β-blocker,
beta-adrenergic receptor blocker; IABP, intra-aortic balloon pump.

| | | NF | LV-HF | BiV-HF |
|---|---|---|---|---|
| | | (n = 5) | (n = 5) | (n = 5) |
| Demographics | Age (yrs), mean ± SEM | 49.0 ± 3.4 | 61.6 ± 1.1 | 61.8 ± 2.2 |
| | median (IQR) | 51.0 (9) | 62 (1) | 60.0 (4) |
| | Gender (% male) | 20 | 60 | 100 |
| Co-Morbidities | Hypertension (%) | 100 | 40 | 40 |
| & Past Medical | Diabetes Mellitus (%) | 20 | 20 | 20 |
| History | Atrial Fibrillation (%) | 0 | 20 | 40 |
| | CAD (%) | 0 | 100 | 100 |
| | h/o MI (%) | 0 | 80 | 80 |
| | h/o CABG (%) | 0 | 100 | 100 |
| Medications | ACEi/ARB/MRA (%) | 0 | 80 | 80 |
| | β-blocker (%) | 80 | 80 | 20 |
| | Hydralazine (%) | 0 | 20 | 0 |
| | Nitrate (%) | 0 | 100 | 80 |
| | Digoxin (%) | 0 | 60 | 60 |
| | Diuretic (%) | 20 | 60 | 60 |
| Circulatory | Inotropic support (%) | 0 | 40 | 100 |
| Support | IABP (%) | 0 | 20 | 20 |
| Echo | LVEF (%) | 57.5 ± 4.5 | 25.0 ± 3.4 | 12 ± 2.3 |
| Parameters | LVEDD (cm) | 4.4 ± 0.3 | 6.6 ± 0.4 | 7.0 ± 0.4 |
| | MR Severity (n) | | | |
| | None | 4 | 2 | 0 |
| | Mild | 1 | 2 | 2 |
| | Moderate | 0 | 1 | 3 |
| | Severe | 0 | 0 | 0 |
| | TR Severity (n) | | | |
| | None | 3 | 4 | 1 |
| | Mild | 2 | 1 | 2 |
| | Moderate | 0 | 0 | 1 |
| | Severe | 0 | 0 | 1 |

TABLE 4
Hemodynamic parameters of BiV-HF and LV-HF patient cohorts. RA, right atrial
pressure; PASP, pulmonary artery systolic pressure; PCWP, pulmonary capillary wedge
pressure; MAP, mean arterial pressure; CI, cardiac index; SBP, systolic blood pressure;
DBP, diastolic blood pressure. ANOVA p value shown in table. *p < 0.0001 vs. NF;
**p < 0.05 vs. NF; ***p < 0.001 vs. NF; ^{†}p < 0.001 vs. LV-HF; ^{‡}p < 0.001 vs. LV-HF;
^{§}p < 0.05 vs. LV-HF; ^{#}p < 0.1 vs. LV-HF on Tukey's multiple comparison test.

| | NF | LV-HF | BiV-HF | P value |
|---|---|---|---|---|
| RA (mmHg) | 7.2 ± 1.7 | 4.0 ± 0.5 | 25.8 ± 1.7*^{†} | <0.0001 |
| PASP (mmHg) | 27.4 ± 2.2 | 26.0 ± 1.5 | 67.0 ± 1.8*^{†} | <0.0001 |
| PCWP (mmHg) | 14.8 ± 2.2 | 10.6 ± 1.1 | 25.6 ± 2.6**^{‡} | <0.001 |
| RA:PCWP | 0.46 ± 0.07 | 0.38 ± 0.03 | 1.05 ± 0.11***^{‡} | <0.001 |
| MAP:RA | 17.2 ± 4.5 | 22.3 ± 2.9 | 2.8 ± 0.3**^{§} | <0.01 |
| CI (L/min/m2 ) | 3.7 ± 0.2 | 3.3 ± 0.4 | 2.2 ± 0.2**^{§} | <0.01 |
| SBP (mmHg) | 130.6 ± 6.2 | 121.8 ± 5.4 | 99.4 ± 6.6**^{#} | <0.01 |
| DBP (mmHg) | 79.6 ± 4.5 | 64.2 ± 7.2 | 56.8 ± 4.7** | 0.04 |
| MAP (mmHg) | 96.6 ± 4.8 | 83.4 ± 5.6 | 71.0 ± 3.6** | <0.01 |

### Example 3

### Transcriptomic Analysis Identifies a Gene Module Uniquely Associated with RVF

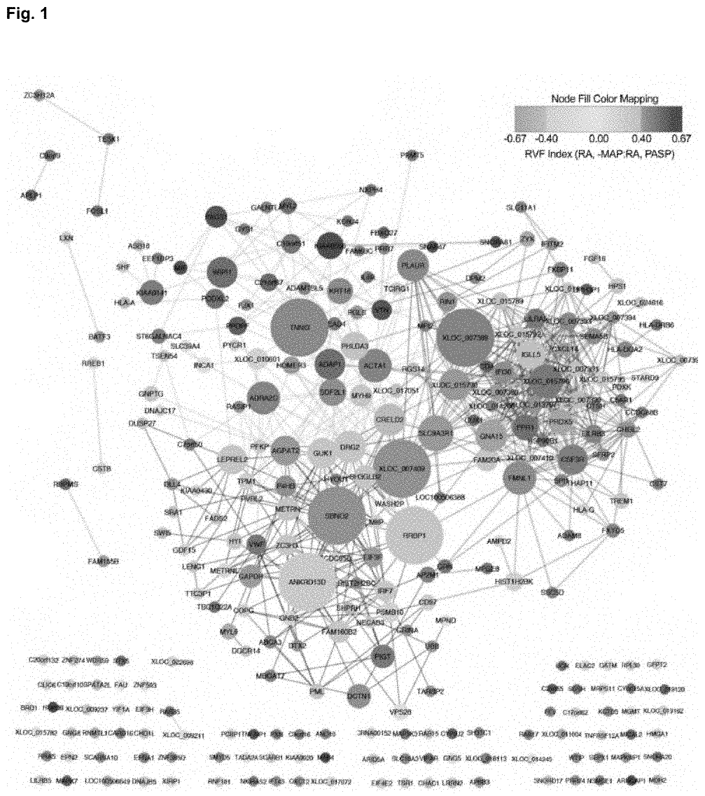

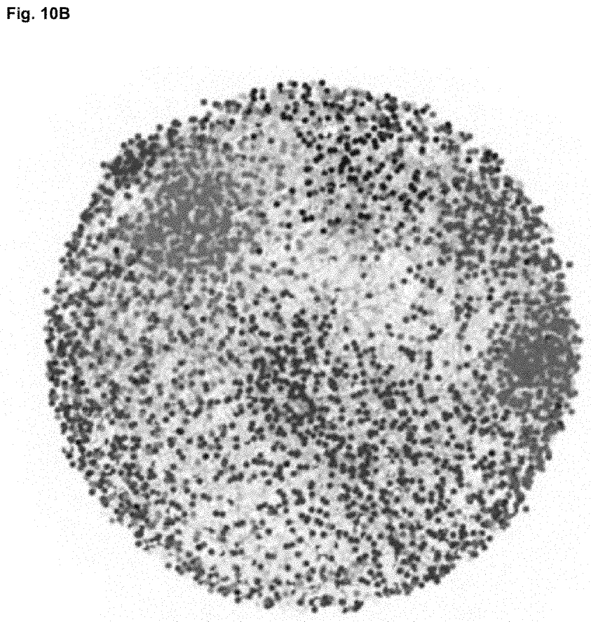

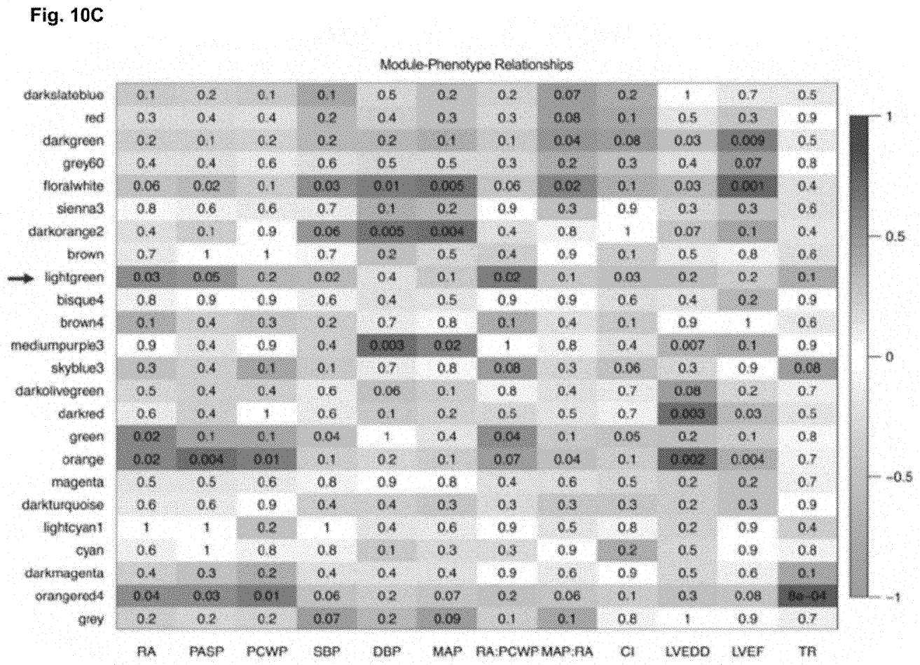

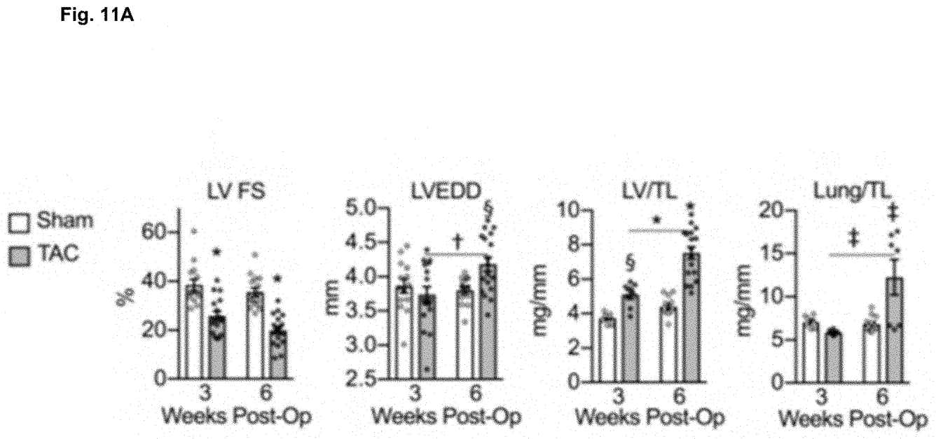

We used WGCNA to identify genetic pathways and groups of genes that distinguish the RV from the whole heart (Langfelder and Horvath, 2008). Using only genes that were expressed (average FPKM>1) and variable (coefficient of variation >10% across all cohorts) in the RV, we partitioned 13,613 transcripts into 23 RV-derived gene modules (FIGS. 10A and 10B). Each module was defined by a tighter clustering coefficient compared to the network as a whole. We examined the correlation of the eigengene for each of the 23 RV-derived network modules with hemodynamic indices of RVF. Consequently, we identified one module that correlated significantly with elevated RAP, elevated RA:PCWP, decreased SBP, and decreased CI (FIG. 10C). This RV-derived, RVF-associated module contained 279 transcripts, of which 245 were protein-coding genes, 30 were novel transcripts, and 4 were non-coding RNAs (1 long intergenic non-coding RNA, 1 pseudogene, 1 regulatory RNA, 1 anti-sense RNA). These 279 transcripts displayed an average of 6.9 connections per transcript (FIG. 1). GeneAnalytics revealed that the module was enriched in genes involved in striated muscle contraction, cytoskeletal signaling, fMLP (N-formyl-Met-Leu-Phe) signaling, receptor tyrosine kinase EphB-EphrinB signaling, oxidative stress response, and protein metabolism (see Data File 1). Other signaling pathways already known to be involved in HF—PKA signaling, PI3K-Akt signaling, Wnt signaling, and AMPK signaling—were also highlighted. Strikingly, the RVF-associated module appeared trilobed in structure. Two of the three lobes had strong, distinct physiological themes—muscle filament sliding and striated muscle contraction (cardiac signaling lobe, see Data File 2); and neutrophil degranulation and innate immune system signaling (innate immunity signaling lobe, see Data File 3). The cardiac signaling lobe was also enriched in genes involved in cytoskeletal signaling, cell death (apoptosis and autophagy), and intracellular membrane transport. The innate immune system signaling lobe was additionally enriched in genes involved in cytokine signaling, cell chemotaxis, and phospholipase C signaling. The third lobe of the RVF-associated module was moderately enriched in genes involved in NOTCH signaling, fMLP pathway, metabolism, calcium homeostasis, and endoplasmic reticulum stress (intracellular signaling lobe, see Data File 4).

Data File 1. GeneAnalytics characterization of RVF-associated module.
GeneAnalytics - Pathway results
Statistics:

| | | |
|---|---|---|
| # Identified genes: 149 | # Matched genes: 52 | # Matched entities: 20 |

Matching score statistics:

| | | |
|---|---|---|
| # High score matches: 2 | # Med score matches: 18 | # Low score matches: 0 |

Results

| | | | # SuperPath | | |
|---|---|---|---|---|---|
| | SuperPath | # SuperPath | Matched | Matched Genes | |
| Score | Name | Total Genes | Genes | (Symbols) | Evidence URL |
| 17.74 | Innate | 2132 | 35 | ADAM8, HLA-A, | http://pathcards. |
| | Immune | | | IFI30, IRF7, | genecards.org/card/ |
| | System | | | FPR1, IFITM2, | innate_immune_system |
| | | | | HLA-G, AGPAT2, | |
| | | | | MIF, UBB, HLA- | |
| | | | | DQA2, PSMB10, | |
| | | | | CSF3R, | |
| | | | | SLC11A1, CTSH, | |
| | | | | FGF18, VWF, | |
| | | | | VTN, DCTN1, | |
| | | | | AP2M1, IL4R, | |
| | | | | FBXO27, | |
| | | | | HSP9061, GRN, | |
| | | | | MYH9, LILRA5, | |
| | | | | TCIRG1, LILRB3, | |
| | | | | ASB18, C5AR1, | |
| | | | | PDXK, PLAUR, | |
| | | | | PML, CDA, | |
| | | | | TREM1 | |
| 15.98 | Striated | 41 | 5 | MYL2, MYL9, | http://pathcards. |
| | Muscle | | | ACTA1, TNNI3, | genecards.org/card/ |
| | Contraction | | | TPM1 | striated_muscle_contraction |
| 12.77 | Interferon | 202 | 8 | HLA-A, IF130, | http://pathcards. |
| | Gamma | | | IRF7, IFITM2, | genecards.org/ |
| | Signaling | | | HLA-G, UBB, | card/ |
| | | | | HLA-DQA2, PML | interferon_gamma_signaling |
| 10.83 | Cytokine | 761 | 15 | HLA-A, IF130, | http://pathcards. |
| | Signaling in | | | IRF7, FPR1, | genecards.org/ |
| | Immune | | | IFITM2, HLA-G, | card/cytokine_sig- |
| | System | | | UBB, HLA-DQA2, | naling_in_immune_system |
| | | | | PSMB10, | |
| | | | | CSF3R, FGF18, | |
| | | | | VWF, IL4R, | |
| | | | | HSP9061, PML | |
| 10.65 | Immune | 91 | 5 | HLA-A, IRF7, | http://pathcards. |
| | Response | | | IFITM2, HLA-G, | genecards.org/card/ |
| | IFN | | | PML | immune_response_ifn_alpha |
| | Alpha/beta | | | | beta_signaling_pathway |
| | Signaling | | | | |
| | Pathway | | | | |
| 10.63 | FMLP | 317 | 9 | UBB, PSMB10, | http://pathcards. |
| | Pathway | | | GNA15, GAPDH, | genecards.org/ |
| | | | | GNB2, DCTN1, | card/fmlp_pathway |
| | | | | GRINA, ACTA1, | |
| | | | | GYS1 | |
| 9.90 | Actin | 341 | 9 | HLA-A, MYL2, | http://pathcards. |
| | Nucleation | | | HLA-G, HLA- | genecards.org/ |
| | By ARP- | | | DQA2, GNA15, | card/ |
| | WASP | | | GNB2, MYL9, | actin_nucleation_by_arp- |
| | Complex | | | ACTA1, MYH9 | wasp_complex |
| 9.86 | MHC Class II | 103 | 5 | IFI30, HLA- | http://pathcards. |
| | Antigen | | | DQA2, CTSH, | genecards.org/ |
| | Presentation | | | DCTN1, AP2M1 | card/ |
| | | | | | mhc_class_ii_antigen_presentation |
| 9.70 | Class I MHC | 823 | 15 | HLA-A, IF130, | http://pathcards. |
| | Mediated | | | HLA-G, UBB, | genecards.org/ |
| | Antigen | | | HLA-DQA2, | card/class_i_mhc_mediated_anti- |
| | Processing | | | PSMB10, CTSH, | gen_processing_and_presentation |
| | and | | | FGF18, DCTN1, | |
| | Presentation | | | AP2M1, FBXO27, | |
| | | | | LILRA5, LILRB3, | |
| | | | | ASB18, TREM1 | |
| 9.51 | Nef-mediates | 29 | 3 | HLA-A, PACS1, | http://pathcards. |
| | Down | | | AP2M1 | genecards.org/ |
| | Modulation of | | | | card/nef-mediates_down_ |
| | Cell Surface | | | | modulation_of_cell_surface_re- |
| | Receptors By | | | | ceptors_by_re- |
| | Recruiting | | | | cruiting_them_to_clathrin_adapters |
| | Them to | | | | |
| | Clathrin | | | | |
| | Adapters | | | | |
| 9.37 | Surfactant | 30 | 3 | ABCA3, | http://pathcards. |
| | Metabolism | | | ADRA2C, CTSH | genecards.org/ |
| | | | | | card/surfactant_metabolism |
| 8.96 | Cytoskeletal | 304 | 8 | KRT18, DCTN1, | http://pathcards. |
| | Signaling | | | MYL9, AP2M1, | genecards.org/ |
| | | | | ACTA1, MYH9, | card/cytoskeletal_signaling |
| | | | | TNNI3, TPM1 | |
| 8.85 | Antigen | 121 | 5 | HLA-A, HLA-G, | http://pathcards. |
| | Processing- | | | UBB, PSMB10, | genecards.org/ |
| | Cross | | | HSP90B1 | card/antigen_processing- |
| | Presentation | | | | cross_presentation |
| 8.75 | Cytoskeleton | 35 | 3 | FPR1, AP2M1, | http://pathcards. |
| | Remodeling_ | | | ACTA1 | genecards.org/ |
| | RalA | | | | card/ |
| | Regulation | | | | cytoskeleton_remodeling_rala_re- |
| | Pathway | | | | gulation_pathway |
| 8.60 | Myoclonic | 10 | 2 | UBB, GYS1 | http://pathcards. |
| | Epilepsy of | | | | genecards.org/ |
| | Lafora | | | | card/myoclonic_epilepsy_of_lafora |
| 8.48 | Cytoskeleton | 187 | 6 | MYL2, VTN, | http://pathcards. |
| | Remodeling | | | MYL9, ACTA1, | genecards.org/card/ |
| | Regulation of | | | MYH9, ZYX | cytoskeleton_remodeling_re- |
| | Actin | | | | gulation_of_actin_cyto- |
| | Cytoskeleton | | | | skeleton_by_rho_gtpases |
| | By Rho | | | | |
| | GTPases | | | | |
| 8.22 | Complement | 82 | 4 | VWF, VTN, | http://pathcards. |
| | and | | | C5AR1, PLAUR | genecards.org/ |
| | Coagulation | | | | card/ |
| | Cascades | | | | complement_and_coagu- |
| | | | | | lation_cascades |
| 8.18 | Immuno- | 135 | 5 | HLA-A, HLA-G, | http://pathcards. |
| | regulatory | | | LILRA5, LILRB3, | genecards.org/ |
| | Interactions | | | TREM1 | card/immuno- |
| | Between A | | | | regulatory_interactions_be- |
| | Lymphoid | | | | tween_a_lymphoid_and_a_non- |
| | and A Non- | | | | lymphoid_cell |
| | Lymphoid | | | | |
| | Cell | | | | |
| 7.93 | PI3K-Akt | 342 | 8 | CSF3R, FGF18, | http://pathcards. |
| | Signaling | | | VWF, GNB2, | genecards.org/ |
| | Pathway | | | VTN, IL4R, | card/pi3k- |
| | | | | HSP9061, GYS1 | akt_signaling_pathway |
| 7.74 | NOTCH2 | 45 | 3 | DTX2, UBB, | http://pathcards. |
| | Activation | | | DLL4 | genecards.org/ |
| | and | | | | card/notch2_activation_and_trans- |
| | Transmission | | | | mission_of_sig- |
| | of Signal to | | | | nal_to_the_nucleus |
| | The Nucleus | | | | |

GeneAnalytics - Function-based Analysis, GO-Biological Process results
Statistics:

| | | |
|---|---|---|
| #Identified genes: 149 | #Matched genes: 41 | #Matched entities: 20 |

Matching score statistics:

| | | |
|---|---|---|
| #High score matches: 2 | #Med score matches: 18 | #Low score matches: 0 |

Results

| | | | # Matched | Matched Genes | |
|---|---|---|---|---|---|
| Score | Name | # Genes | Genes | (Symbols) | Evidence URL |
| 15.86 | Interferon- | 71 | 6 | HLA-A, IFI30, | http://amigo.geneontology.org/ |
| | gamma- | | | IRF7, HLA-G, | amigo/term/GO:0060333 |
| | mediated | | | HLA-DQA2, PML | |
| | Signaling | | | | |
| | Pathway | | | | |
| 13.82 | Neutrophil | 484 | 13 | ADAM8, FPR1, | http://amigo.geneontology.org/ |
| | Degranulation | | | AGPAT2, MIF, | amigo/term/GO:0043312 |
| | | | | SLC11A1, CTSH, | |
| | | | | GRN, TCIRG1, | |
| | | | | LILRB3, C5AR1, | |
| | | | | PDXK, PLAUR, | |
| | | | | CDA | |
| 12.30 | Muscle | 38 | 4 | MYL2, ACTA1, | http://amigo.geneontology.org/ |
| | Filament | | | TNNI3, TPM1 | amigo/term/GO:0030049 |
| | Sliding | | | | |
| 11.49 | Regulation of | 18 | 3 | MYL9, TNNI3, | http://amigo.geneontology.org/ |
| | Muscle | | | TPM1 | amigo/term/GO:0006937 |
| | Contraction | | | | |
| 11.05 | Defense | 20 | 3 | SLC11A1, IL4R, | http://amigo.geneontology.org/ |
| | Response to | | | CCDC88B | amigo/term/GO:0042832 |
| | Protozoan | | | | |
| 10.57 | Negative | 5 | 2 | RGS14, RIN1 | http://amigo.geneontology.org/ |
| | Regulation of | | | | amigo/term/GO:0031914 |
| | Synaptic | | | | |
| | Plasticity | | | | |
| 10.12 | Ventricular | 25 | 3 | MYL2, TNNI3, | http://amigo.geneontology.org/ |
| | Cardiac | | | TPM1 | amigo/term/GO:0055010 |
| | Muscle | | | | |
| | Tissue | | | | |
| | Morphogenesis | | | | |
| 9.66 | Positive | 28 | 3 | AGPAT2, | http://amigo.geneontology.org/ |
| | Regulation of | | | SLC11A1, | amigo/term/GO:0001819 |
| | Cytokine | | | CCDC88B | |
| | Production | | | | |
| 9.61 | Attachment | 7 | 2 | PLAUR, PIGT | http://amigo.geneontology.org/ |
| | of GPI | | | | amigo/term/GO:0016255 |
| | Anchor to | | | | |
| | Protein | | | | |
| 9.34 | Type I | 66 | 4 | HLA-A, IRF7, | http://amigo.geneontology.org/ |
| | Interferon | | | IFITM2, HLA-G | amigo/term/GO:0060337 |
| | Signaling | | | | |
| | Pathway | | | | |
| 9.24 | Positive | 31 | 3 | CTSH, INCA1, | http://amigo.geneontology.org/ |
| | Regulation of | | | PML | amigo/term/GO:2001235 |
| | Apoptotic | | | | |
| | Signaling | | | | |
| | Pathway | | | | |
| 8.90 | Negative | 120 | 5 | MYL2, SFRP2, | http://amigo.geneontology.org/ |
| | Regulation of | | | PML, CCDC85B, | amigo/term/GO:0030308 |
| | Cell Growth | | | CDA | |
| 8.90 | Antigen | 9 | 2 | HLA-A, HLA-G | http://amigo.geneontology.org/ |
| | Processing | | | | amigo/term/GO:0002480 |
| | and | | | | |
| | Presentation | | | | |
| | of Exogenous | | | | |
| | Peptide | | | | |
| | Antigen Via | | | | |
| | MHC Class I, | | | | |
| | TAP- | | | | |
| | independent | | | | |
| 8.41 | Response to | 79 | 4 | SDF2L1, P4HB, | http://amigo.geneontology.org/ |
| | Endoplasmic | | | HSP9061, | amigo/term/GO:0034976 |
| | Reticulum | | | HYOU1 | |
| | Stress | | | | |
| 8.33 | Complement | 11 | 2 | FPR1, C5AR1 | http://amigo.geneontology.org/ |
| | Receptor | | | | amigo/term/GO:0002430 |
| | Mediated | | | | |
| | Signaling | | | | |
| | Pathway | | | | |
| 8.33 | Immunoglobu | 11 | 2 | IRF7, IL4R | http://amigo.geneontology.org/ |
| | lin Mediated | | | | amigo/term/GO:0016064 |
| | Immune | | | | |
| | Response | | | | |
| 8.09 | Vacuolar | 12 | 2 | SLC11A1, | http://amigo.geneontology.org/ |
| | Acidification | | | TCIRG1 | amigo/term/GO:0007035 |
| 7.86 | Phospholipase | 13 | 2 | GNA15, | http://amigo.geneontology.org/ |
| | C-activating | | | SLC9A3R1 | amigo/term/GO:0060158 |
| | Dopamine | | | | |
| | Receptor | | | | |
| | Signaling | | | | |
| | Pathway | | | | |
| 7.65 | Cardiac | 46 | 3 | MYL2, TNNI3, | http://amigo.geneontology.org/ |
| | Muscle | | | TPM1 | amigo/term/GO:0060048 |
| | Contraction | | | | |
| 7.63 | Antigen | 92 | 4 | IFI30, HLA- | http://amigo.geneontology.org/ |
| | Processing | | | DQA2, DCTN1, | amigo/term/GO:0019886 |
| | and | | | AP2M1 | |
| | Presentation | | | | |
| | of Exogenous | | | | |
| | Peptide | | | | |
| | Antigen Via | | | | |
| | MHC Class II | | | | |

GeneAnalytics - Function-based Analysis, GO-Molecular Process results
Statistics:

| | | |
|---|---|---|
| # Identified genes: 149 | # Matched genes: 92 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 9.74 | Integrin | 105 | 5 | SFRP2, VWF, | http://amigo.geneontology.org/ |
| | Binding | | | VTN, P4HB, | amigo/term/GO:005178 |
| | | | | MFGE8 | |
| 9.23 | Myosin | 8 | 2 | MYL2, MYL9 | http://amigo.geneontology.org/ |
| | Heavy Chain | | | | amigo/term/GO:0032036 |
| | Binding | | | | |
| 8.90 | Complement | 9 | 2 | FPR1, C5AR1 | http://amigo.geneontology.org/ |
| | Receptor | | | | amigo/term/GO:0004875 |
| | Activity | | | | |
| 7.86 | Protein | 9013 | 83 | ADAM8, HLA-A, | http://amigo.geneontology.org/ |
| | Binding | | | AMPD2, IFI30, | amigo/term/GO:0005515 |
| | | | | IRF7, SNAP47, | |
| | | | | DTX2, FPR1, | |
| | | | | MYL2, MBOAT7, | |
| | | | | TSEN54, | |
| | | | | ADRA2C, | |
| | | | | SSC5D, KRT18, | |
| | | | | MIF, ADAP1, | |
| | | | | UBB, MIIP, | |
| | | | | EIF3F, PSMB10, | |
| | | | | HOMER3, | |
| | | | | CSF3R, PRMT5, | |
| | | | | SH3GLB2, | |
| | | | | GDF15, HPS1, | |
| | | | | CRELD2, CTSH, | |
| | | | | SHPRH, GAPDH, | |
| | | | | VPS28, CST7, | |
| | | | | PYCR1, VWF, | |
| | | | | GNB2, VTN, | |
| | | | | SRA1, CXCL14, | |
| | | | | DCTN1, KCNJ4, | |
| | | | | PACS1, FAM20A, | |
| | | | | NECAB3, | |
| | | | | AP2M1, | |
| | | | | DGCR14, | |
| | | | | SLC9A3R1, | |
| | | | | ACTA1, P4HB, | |
| | | | | SWI5, DLL4, | |
| | | | | RGS14, FMNL1, | |
| | | | | RINI, IL4R, | |
| | | | | TBC1D22A, | |
| | | | | LENG1, DPM2, | |
| | | | | FBX027, DRG2, | |
| | | | | HSP9061, | |
| | | | | FXYD5, GRN, | |
| | | | | TARBP2, | |
| | | | | KIAA0141, | |
| | | | | THAP11, MYH9, | |
| | | | | TEAD4, INCA1, | |
| | | | | HYI, LILRB3, | |
| | | | | ZYX, C7orf50, | |
| | | | | GYS1, PLAUR, | |
| | | | | TNNI3, SPI1, | |
| | | | | PGLS, PML, | |
| | | | | TPM1, PIGT, | |
| | | | | CCDC85B, | |
| | | | | PODXL2, CDA | |
| 7.66 | GTPase | 14 | 2 | RGS14, FMNL1 | http://amigo.geneontology.org/ |
| | Activating | | | | amigo/term/GO:0032794 |
| | Protein | | | | |
| | Binding | | | | |
| 7.65 | Structural | 46 | 3 | MYL2, MYL9, | http://amigo.geneontology.org/ |
| | Constituent | | | TPM1 | amigo/term/GO:0008307 |
| | of Muscle | | | | |
| 7.09 | Metal | 1 | 1 | SLC11A1 | http://amigo.geneontology.org/ |
| | Ion:proton | | | | amigo/term/GO:0051139 |
| | Antiporter | | | | |
| | Activity | | | | |
| 7.09 | RNA | 1 | 1 | PRDX5 | http://amigo.geneontology.org/ |
| | Polymerase | | | | amigo/term/GO:0001016 |
| | III Regulatory | | | | |
| | Region DNA | | | | |
| | Binding | | | | |
| 7.09 | Phenylpyruvate | 1 | 1 | MIF | http://amigo.geneontology.org/ |
| | Tautomerase | | | | amigo/term/GO:0050178 |
| | Activity | | | | |
| 7.09 | Complement | 1 | 1 | C5AR1 | http://amigo.geneontology.org/ |
| | Component | | | | amigo/term/GO:0001856 |
| | C5a Binding | | | | |
| 7.09 | Linoleoyl- | 1 | 1 | FADS2 | http://amigo.geneontology.org/ |
| | CoA | | | | amigo/term/GO:0016213 |
| | Desaturase | | | | |
| | Activity | | | | |
| 7.09 | TAP Binding | 1 | 1 | HLA-A | http://amigo.geneontology.org/ |
| | | | | | amigo/term/GO:0046977 |
| 7.09 | Peptidyl- | 1 | 1 | GAPDH | http://amigo.geneontology.org/ |
| | cysteine S- | | | | amigo/term/GO:0035605 |
| | nitrosylase | | | | |
| | Activity | | | | |
| 7.09 | (alpha-N- | 1 | 1 | ST6GALNAC4 | http://amigo.geneontology.org/ |
| | acetyl- | | | | amigo/term/GO:0047290 |
| | neuraminyl- | | | | |
| | 2,3-beta- | | | | |
| | galactosyl- | | | | |
| | 1,3)-N-acetyl- | | | | |
| | galactosaminide | | | | |
| | 6-alpha- | | | | |
| | sialyltransferase | | | | |
| | Activity | | | | |
| 7.09 | Hydroxypyruvate | 1 | 1 | HYI | http://amigo.geneontology.org/ |
| | Isomerase | | | | amigo/term/GO:0008903 |
| | Activity | | | | |
| 7.09 | HLA-A | 1 | 1 | CTSH | http://amigo.geneontology.org/ |
| | Specific | | | | amigo/term/GO:0030108 |
| | Activating | | | | |
| | MHC Class I | | | | |
| | Receptor | | | | |
| | Activity | | | | |
| 7.09 | Granulocyte | 1 | 1 | CSF3R | http://amigo.geneontology.org/ |
| | Colony- | | | | amigo/term/GO:0051916 |
| | stimulating | | | | |
| | Factor | | | | |
| | Binding | | | | |
| 7.09 | Pyridoxal | 1 | 1 | PDXK | http://amigo.geneontology.org/ |
| | Kinase | | | | amigo/term/GO:0008478 |
| | Activity | | | | |
| 7.09 | Urokinase | 1 | 1 | PLAUR | http://amigo.geneontology.org/ |
| | Plasminogen | | | | amigo/term/GO:0030377 |
| | Activator | | | | |
| | Receptor | | | | |
| | Activity | | | | |
| 6.96 | Low-density | 18 | 2 | AP2M1, | http://amigo.geneontology.org/ |
| | Lipoprotein | | | HSP90B1 | amigo/term/GO:0050750 |
| | Particle | | | | |
| | Receptor | | | | |
| | Binding | | | | |

GeneAnalytics - Function-based Analysis, Phenotype results
Statistics:

| | | |
|---|---|---|
| # Identified genes: 149 | # Matched genes: 31 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 11.08 | Increased | 241 | 8 | FPR1, HLA-G, | http://www.informatics.jax.org/ |
| | Susceptibility | | | SLC11A1, VWF, | searches/ |
| | to Bacterial | | | GRN, C5AR1, | Phat.cgi?id=MP:0002412 |
| | Infection | | | PLAUR, PML | |
| 10.57 | Decreased | 5 | 2 | ACTA1, GYS1 | http://www. informatics.jax.org/ |
| | Skeletal | | | | searches/ |
| | Muscle | | | | Phat.cgi?id=MP:0010399 |
| | Glycogen | | | | |
| | Level | | | | |
| 10.12 | Abnormal | 25 | 3 | MBOAT7, | http://www.informatics.jax.org/ |
| | Neurite | | | SEMA5B, GRN | searches/ |
| | Morphology | | | | Phat.cgi?id=MP:0008415 |
| 10.12 | Decreased | 25 | 3 | CSF3R, SPI1, | http://www.informatics.jax.org/ |
| | Granulocyte | | | PML | searches/ |
| | Number | | | | Phat.cgi?id=MP:0000334 |
| 10.05 | Abnormal | 6 | 2 | CSF3R, SPI1 | http://www.informatics.jax.org/ |
| | Monocyte | | | | searches/ |
| | Morphology | | | | Phat.cgi?id=MP:0002620 |
| 9.42 | Abnormal | 65 | 4 | FADS2, VWF, | http://www.informatics.jax.org/ |
| | Blood | | | VTN, MYH9 | searches/ |
| | Coagulation | | | | Phat.cgi?id=MP:0002551 |
| 9.24 | Osteopetrosis | 31 | 3 | SBNO2, TCIRG1, | http://www. informatics.jax.org/ |
| | | | | SPI1 | searches/ |
| | | | | | Phat.cgi?id=MP:0000067 |
| 9.11 | Increased | 69 | 4 | FADS2, HPS1, | http://www.informatics.jax.org/ |
| | Bleeding | | | VWF, MYH9 | searches/ |
| | Time | | | | Phat.cgi?id=MP:0005606 |
| 8.90 | Abnormal | 9 | 2 | CSF3R, SPI1 | http://www.informatics.jax.org/ |
| | Neutrophil | | | | searches/ |
| | Differentiation | | | | Phat.cgi?id=MP:0002415 |
| 8.67 | Abnormal | 182 | 6 | FADS2, HPS1, | http://www.informatics.jax.org/ |
| | Macrophage | | | IL4R, GRN, | searches/ |
| | Physiology | | | TCIRG1, PLAUR | Phat.cgi?id=MP:0002451 |
| 8.52 | Increased | 37 | 3 | SNAP47, | http://www.informatics.jax.org/ |
| | Bone Mineral | | | FKBP11, TCIRG1 | searches/ |
| | Content | | | | Phat.cgi?id=MP:0010123 |
| 8.31 | Abnormal | 39 | 3 | FADS2, INCA1, | http://www.informatics.jax.org/ |
| | Spleen White | | | TCIRG1 | searches/ |
| | Pulp | | | | Phat.cgi?id=MP:0002357 |
| | Morphology | | | | |
| 8.31 | Increased | 39 | 3 | MYH9, INCA1, | http://www.informatics.jax.org/ |
| | Megakaryocyte | | | TCIRG1 | searches/ |
| | Cell | | | | Phat.cgi?id=MP:0008254 |
| | Number | | | | |
| 8.09 | Abnormal | 12 | 2 | DLL4, GYS1 | http://www.informatics.jax.org/ |
| | Pericardial | | | | searches/ |
| | Cavity | | | | Phat.cgi?id=MP:0012501 |
| | Morphology | | | | |
| 8.09 | Increased B- | 12 | 2 | LILRB3, SPI1 | http://www.informatics.jax.org/ |
| | 1 B Cell | | | | searches/ |
| | Number | | | | Phat.cgi?id=MP:0004977 |
| 7.83 | Decreased | 44 | 3 | AGPAT2, SRA1, | http://www.informatics.jax.org/ |
| | Brown | | | ACTA1 | searches/ |
| | Adipose | | | | Phat.cgi?id=MP:0001780 |
| | Tissue | | | | |
| | Amount | | | | |
| 7.66 | Abnormal | 14 | 2 | DLL4, DNAAF3 | http://www.informatics.jax.org/ |
| | Vein | | | | searches/ |
| | Morphology | | | | Phat.cgi?id=MP:0002725 |
| 7.65 | Abnormal | 46 | 3 | INCA1, TCIRG1, | http://www.informatics.jax.org/ |
| | Spleen B Cell | | | MFGE8 | searches/ |
| | Follicle | | | | Phat.cgi?id=MP:0008470 |
| | Morphology | | | | |
| 7.58 | Enlarged | 93 | 4 | FADS2, AGPAT2, | http://www.informatics.jax.org/ |
| | Liver | | | KRT18, SPI1 | searches/ |
| | | | | | Phat.cgi?id=MP:0000599 |
| 7.47 | Abnormal | 15 | 2 | AGPAT2, | http://www.informatics.jax.org/ |
| | Dentin | | | TCIRG1 | searches/ |
| | Morphology | | | | Phat.cgi?id=MP:0002818 |

Data File 2. GeneAnalytics characterization of “Cardiac Lobe” of RVF-associated module.
GeneAnalytics - Pathway results
Statistics:

| | | |
|---|---|---|
| # Identified genes: 35 | # Matched genes: 16 | # Matched entities: 20 |

Matching score statistics:

| | | |
|---|---|---|
| # High score matches: 1 | # Med score matches: 19 | # Low score matches: 0 |

Results

| | | | # SuperPath | | |
|---|---|---|---|---|---|
| | SuperPath | # SuperPath | Matched | Matched Genes | |
| Score | Name | Total Genes | Genes | (Symbols) | Evidence URL |
| 14.23 | Striated | 41 | 3 | MYL2, ACTA1, | http://pathcards. |
| | Muscle | | | TNN13 | genecards.org/ |
| | Contraction | | | | card/ |
| | | | | | striated_muscle_contraction |
| 9.72 | Nef-mediates | 29 | 2 | HLA-A, PACS1 | http://pathcards. |
| | Down | | | | genecards.org/ |
| | Modulation of | | | | card/nef- |
| | Cell Surface | | | | mediates_down_mod- |
| | Receptors By | | | | ulation_of_cell_sur- |
| | Recruiting | | | | face_receptors_by_re- |
| | Them to | | | | cruiting_them_to_clathrin_adapters |
| | Clathrin | | | | |
| | Adapters | | | | |
| 9.35 | Inflammatory | 33 | 2 | VTN, IL4R | http://pathcards. |
| | Response | | | | genecards.org/ |
| | Pathway | | | | card/inflammatory_re- |
| | | | | | sponse_pathway |
| 8.74 | RhoA | 41 | 2 | MYL2, ACTA1 | http://pathcards. |
| | Signaling | | | | genecards.org/ |
| | Pathway | | | | card/rhoa_signaling_pathway |
| 8.17 | Glycogen | 2 | 1 | GYS1 | http://pathcards. |
| | Storage | | | | genecards.org/card/ |
| | Disease Type | | | | glycogen_stor- |
| | XV (GYG1) | | | | age_disease_type_xv_(gyg1) |
| 8.01 | Cardiac | 53 | 2 | MYL2, TNNI3 | http://pathcards. |
| | Progenitor | | | | genecards.org/ |
| | Differentiation | | | | card/cardiac_pro- |
| | | | | | genitor_differentiation |
| 7.91 | Cytoskeleton | 187 | 3 | ACTA1 | http://pathcards. |
| | Remodeling | | | | genecards.org/ |
| | Regulation of | | | | card/cytoskeleton_remodeling_re- |
| | MYL2, VTN, | | | | gulation_of_actin_cyto- |
| | Actin | | | | skeleton_by_rho_gtpases |
| | Cytoskeleton | | | | |
| | By Rho | | | | |
| | GTPases | | | | |
| 7.18 | Lysine | 4 | 1 | PYCR1 | http://pathcards. |
| | Degradation | | | | genecards.org/ |
| | II (pipecolate | | | | card/lysine_degra- |
| | Pathway) | | | | dation_ii_(pipecolate_pathway) |
| 7.07 | Cardiac | 231 | 3 | MYL2, KCNJ4, | http://pathcards. |
| | Conduction | | | TNNI3 | genecards.org/ |
| | | | | | card/cardiac_conduction |
| 6.94 | Cardiac | 78 | 2 | MYL2, TNNI3 | http://pathcards. |
| | Muscle | | | | genecards.org/card/ |
| | Contraction | | | | cardiac_muscle_contraction |
| 6.94 | Synthesis of | 78 | 2 | ST6GALNAC4, | http://pathcards. |
| | Substrates in | | | DPM2 | genecards.org/ |
| | N-glycan | | | | card/ |
| | Biosythesis | | | | synthesis_of_substrates_in _n- |
| | | | | | glycan_biosythesis |
| 6.59 | Pentose | 6 | 1 | PGLS | http://pathcards. |
| | Phosphate | | | | genecards.org/card/ |
| | Pathway | | | | pentose_phosphate_path- |
| | (Erythrocyte) | | | | way_(erythrocyte) |
| 6.54 | Apoptosis | 90 | 2 | KRT18, ACTA1 | http://pathcards. |
| | and Survival | | | | genecards.org/card/ |
| | Caspase | | | | apoptosis_and_sur- |
| | Cascade | | | | vival_caspase_cascade |
| 6.37 | Mesenchymal | 96 | 2 | HLA-A, TNNI3 | http://pathcards. |
| | Stem Cell | | | | genecards.org/ |
| | Differentiation | | | | mesenchymal_stem_cell_dif- |
| | Pathways | | | | ferentiation_path- |
| | and Lineage- | | | | ways_and_lineage- |
| | specific | | | | specific_markers |
| | Markers | | | | |
| 6.28 | Dilated | 99 | 2 | MYL2, TNNI3 | http://pathcards. |
| | Cardiomyopathy | | | | genecards.org/card/ |
| | | | | | dilated_cardiomyopathy |
| 6.10 | Senescence | 106 | 2 | VTN, SLC39A4 | http://pathcards. |
| | and | | | | genecards.org/card/ |
| | Autophagy in | | | | senescence_and_auto- |
| | Cancer | | | | phagy_in_cancer |
| 6.00 | Cytoskeletal | 304 | 3 | KRT18, ACTA1, | http://pathcards. |
| | Signaling | | | TNNI3 | genecards.org/card/ |
| | | | | | cytoskeletal_signaling |
| 5.86 | Myoclonic | 10 | 1 | GYS1 | http://pathcards. |
| | Epilepsy of | | | | genecards.org/card/ |
| | Lafora | | | | myoclonic_epilepsy_of_lafora |
| 5.85 | O-linked | 116 | 2 | ADAMTSL5, | http://pathcards. |
| | Glycosylation | | | ST6GALNAC4 | genecards.org/card/o- |
| | | | | | linked_glycosylation |
| 5.72 | VEGF | 122 | 2 | VTN, ACTA1 | http://pathcards. |
| | Pathway | | | | genecards.org/card/ |
| | (Qiagen) | | | | vegf_pathway_(qiagen) |

GeneAnalytics - Function-based Analysis, GO-Biological Process results
Statistics:

| | | |
|---|---|---|
| # Identified genes: 35 | # Matched genes: 13 | # Matched entities: 20 |

Matching score statistics:

| | | |
|---|---|---|
| # High score matches: 1 | # Med score matches: 19 | # Low score matches: 0 |

Results

| | | | # Matched | Matched Genes | |
|---|---|---|---|---|---|
| Score | Name | # Genes | Genes | (Symbols) | Evidence URL |
| 14.55 | Muscle | 38 | 3 | MYL2, ACTA1, | http://amigo.geneontology.org/ |
| | Filament | | | TNNI3 | amigo/term/GO:0030049 |
| | Sliding | | | | |
| 11.60 | Heart | 15 | 2 | MYL2, TNNI3 | http://amigo.geneontology.org/ |
| | Contraction | | | | amigo/term/GO:0060047 |
| 10.14 | Ventricular | 25 | 2 | MYL2, TNNI3 | http://amigo.geneontology.org/ |
| | Cardiac | | | | amigo/term/GO:0055010 |
| | Muscle | | | | |
| | Tissue | | | | |
| | Morphogenesis | | | | |
| 9.35 | Liver | 33 | 2 | PRMT5, VTN | http://amigo.geneontology.org/ |
| | Regeneration | | | | amigo/term/GO:0097421 |
| 9.17 | Regulation of | 1 | 1 | TNNI3 | http://amigo.geneontology.org/ |
| | Systemic | | | | amigo/term/GO:0001980 |
| | Arterial Blood | | | | |
| | Pressure By | | | | |
| | Ischemic | | | | |
| | Conditions | | | | |
| 9.17 | Positive | 1 | 1 | MIF | http://amigo.geneontology.org/ |
| | Regulation of | | | | amigo/term/GO:0061081 |
| | Myeloid | | | | |
| | Leukocyte | | | | |
| | Cytokine | | | | |
| | Production | | | | |
| | Involved in | | | | |
| | Immune | | | | |
| | Response | | | | |
| 9.17 | Positive | 1 | 1 | MIF | http://amigo.geneontology.org/ |
| | Regulation of | | | | amigo/term/GO:0061078 |
| | Prostaglandin | | | | |
| | Secretion | | | | |
| | Involved in | | | | |
| | Immune | | | | |
| | Response | | | | |
| 9.17 | Golgi to | 1 | 1 | KRT18 | http://amigo.geneontology.org/ |
| | Plasma | | | | amigo/term/GO:0043000 |
| | Membrane | | | | |
| | CFTR Protein | | | | |
| | Transport | | | | |
| 9.17 | Muscle Cell | 1 | 1 | MYL2 | http://amigo.geneontology.org/ |
| | Fate | | | | amigo/term/GO:0042694 |
| | Specification | | | | |
| 9.17 | Positive | 1 | 1 | PRMT5 | http://amigo.geneontology.org/ |
| | Regulation of | | | | amigo/term/GO:1904992 |
| | Adenylate | | | | |
| | Cyclase- | | | | |
| | inhibiting | | | | |
| | Dopamine | | | | |
| | Receptor | | | | |
| | Signaling | | | | |
| | Pathway | | | | |
| 8.60 | Protein N- | 43 | 2 | ST6GALNAC4, | http://amigo.geneontology.org/ |
| | linked | | | DPM2 | amigo/term/GO:0018279 |
| | Glycosylation | | | | |
| | Via | | | | |
| | Asparagine | | | | |
| 8.41 | Cardiac | 46 | 2 | MYL2, TNNI3 | http://amigo.geneontology.org/ |
| | Muscle | | | | amigo/term/GO:0060048 |
| | Contraction | | | | |
| 8.17 | Skeletal | 2 | 1 | ACTA1 | http://amigo.geneontology.org/ |
| | Muscle Fiber | | | | amigo/term/GO:0043503 |
| | Adaptation | | | | |
| 8.17 | Vesicle | 2 | 1 | WIPI1 | http://amigo.geneontology.org/ |
| | Targeting, | | | | amigo/term/GO:0048203 |
| | Trans-Golgi | | | | |
| | to Endosome | | | | |
| 8.17 | Production of | 2 | 1 | IL4R | http://am igo. geneontology.org/ |
| | Molecular | | | | amigo/term/GO:0002532 |
| | Mediator | | | | |
| | Involved in | | | | |
| | Inflammatory | | | | |
| | Response | | | | |
| 8.17 | Smooth | 2 | 1 | VTN | http://amigo.geneontology.org/ |
| | Muscle Cell- | | | | amigo/term/GO:0061302 |
| | matrix | | | | |
| | Adhesion | | | | |
| 8.17 | Cellular | 2 | 1 | SLC39A4 | http://amigo.geneontology.org/ |
| | Response to | | | | amigo/term/GO:0034224 |
| | Zinc Ion | | | | |
| | Starvation | | | | |
| 7.59 | Negative | 3 | 1 | IL4R | http://amigo.geneontology.org/ |
| | Regulation of | | | | amigo/term/GO:0045626 |
| | T-helper 1 | | | | |
| | Cell | | | | |
| | Differentiation | | | | |
| 7.59 | Pentose- | 3 | 1 | PGLS | http://amigo.geneontology.org/ |
| | phosphate | | | | amigo/term/GO:0009051 |
| | Shunt, | | | | |
| | Oxidative | | | | |
| | Branch | | | | |
| 7.18 | Dolichol | 4 | 1 | DPM2 | http://amigo.geneontology.org/ |
| | Metabolic | | | | amigo/term/GO:0019348 |
| | Process | | | | |

GeneAnalytics - Function-based Analysis, GO-Molecular Process results
Statistics:

| | | |
|---|---|---|
| # Identified genes: 35 | # Matched genes: 15 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 9.17 | TAP Binding | 1 | 1 | HLA-A | http://amigo.geneontology.org/ |
| | | | | | amigo/term/GO:0046977 |
| 9.17 | (alpha-N- | 1 | 1 | ST6GALNAC4 | http://amigo.geneontology.org/ |
| | acetyl- | | | | amigo/term/GO:0047290 |
| | neuraminyl- | | | | |
| | 2,3-beta- | | | | |
| | galactosyl- | | | | |
| | 1,3)-N-acetyl- | | | | |
| | galactosaminide | | | | |
| | 6-alpha- | | | | |
| | sialyltransferase | | | | |
| | Activity | | | | |
| 9.17 | Phenylpyruvate | 1 | 1 | MIF | http://amigo.geneontology.org/ |
| | Tautomerase | | | | amigo/term/GO:0050178 |
| | Activity | | | | |
| 8.17 | Glycogen | 2 | 1 | GYS1 | http://amigo.geneontology.org/ |
| | (starch) | | | | amigo/term/GO:0004373 |
| | Synthase | | | | |
| | Activity | | | | |
| 8.17 | Glycogen | 2 | 1 | GYS1 | http://amigo.geneontology.org/ |
| | Synthase | | | | amigo/term/GO:0061547 |
| | Activity, | | | | |
| | Transferring | | | | |
| | Glucose-1- | | | | |
| | phosphate | | | | |
| 8.17 | 6- | 2 | 1 | PGLS | http://amigo.geneontology.org/ |
| | phosphoglucono- | | | | amigo/term/GO:0017057 |
| | lactonase | | | | |
| | Activity | | | | |
| 8.17 | Interleukin-4 | 2 | 1 | IL4R | http://amigo.geneontology.org/ |
| | Receptor | | | | amigo/term/GO:0004913 |
| | Activity | | | | |
| 7.69 | Receptor | 398 | 4 | HLA-A, MIF, | http://amigo.geneontology.org/ |
| | Binding | | | WIPI1, NXPH4 | amigo/term/GO:0005102 |
| 7.59 | Dopachrome | 3 | 1 | MIF | http://amigo.geneontology.org/ |
| | Isomerase | | | | amigo/term/GO:0004167 |
| | Activity | | | | |
| 7.59 | Dolichyl- | 3 | 1 | DPM2 | http://amigo.geneontology.org/ |
| | phosphate | | | | amigo/term/GO:0004582 |
| | Beta-D- | | | | |
| | mannosyltransferase | | | | |
| | Activity | | | | |
| 7.59 | Troponin C | 3 | 1 | TNNI3 | http://amigo.geneontology.org/ |
| | Binding | | | | amigo/term/GO:0030172 |
| 7.18 | Troponin T | 4 | 1 | TNNI3 | http://amigo.geneontology.org/ |
| | Binding | | | | amigo/term/GO:0031014 |
| 7.18 | Pyrroline-5- | 4 | 1 | PYCR1 | http://amigo.geneontology.org/ |
| | carboxylate | | | | amigo/term/GO:0004735 |
| | Reductase | | | | |
| | Activity | | | | |
| 7.18 | Histone | 4 | 1 | PRMT5 | http://amigo.geneontology.org/ |
| | Methyltransferase | | | | amigo/term/GO:0044020 |
| | Activity | | | | |
| | (H4-R3 | | | | |
| | Specific) | | | | |
| 7.18 | Protein- | 4 | 1 | PRMT5 | http://amigo.geneontology.org/ |
| | arginine | | | | amigo/term/GO:0035243 |
| | Omega-N | | | | |
| | Symmetric | | | | |
| | Methyltransfe | | | | |
| | rase Activity | | | | |
| 6.86 | Inositol | 5 | 1 | ADAP1 | http://amigo.geneontology.org/ |
| | 1,3,4,5 | | | | amigo/term/GO:0043533 |
| | Tetrakis- | | | | |
| | phosphate | | | | |
| | Binding | | | | |
| 6.59 | Histone- | 6 | 1 | PRMT5 | http://amigo.geneontology.org/ |
| | arginine N- | | | | amigo/term/GO:0008469 |
| | methyl- | | | | |
| | transferase | | | | |
| | Activity | | | | |
| 6.59 | Alpha-N- | 6 | 1 | ST6GALNAC4 | http://amigo.geneontology.org/ |
| | acetylgalacto | | | | amigo/term/GO:0001665 |
| | saminide | | | | |
| | Alpha-2,6- | | | | |
| | sialyltransfer | | | | |
| | ase Activity | | | | |
| 6.18 | Myosin | 8 | 1 | MYL2 | http://amigo.geneontology.org/ |
| | Heavy Chain | | | | amigo/term/GO:0032036 |
| | Binding | | | | |
| 5.86 | G-protein | 10 | 1 | KCNJ4 | http://amigo.geneontology.org/ |
| | Activated | | | | amigo/term/GO:0015467 |
| | Inward | | | | |
| | Rectifier | | | | |
| | Potassium | | | | |
| | Channel | | | | |
| | Activity | | | | |

GeneAnalytics - Function-based Analysis, Phenotype results
Statistics:

| | | |
|---|---|---|
| # Identified genes: 35 | # Matched genes: 9 | # Matched entities: 20 |

Matching score statistics:

| | | |
|---|---|---|
| # High score matches: 1 | # Med score matches: 19 | # Low score matches: 0 |

Results

| | | | # Matched | Matched Genes | |
|---|---|---|---|---|---|
| Score | Name | # Genes | Genes | (Symbols) | Evidence URL |
| 14.75 | Decreased | 5 | 2 | ACTA1, GYS1 | http://www.informatics.jax.org/ |
| | Skeletal | | | | searches/ |
| | Muscle | | | | Phat.cgi?id=MP:0010399 |
| | Glycogen | | | | |
| | Level | | | | |
| 10.03 | Thick | 26 | 2 | SLC39A4, TNNI3 | http://www.informatics.jax.org/ |
| | Interventricular | | | | searches/ |
| | Septum | | | | Phat.cgi?id=MP:0010724 |
| 9.78 | Increased | 118 | 3 | MYL2, GYS1, | http://www.informatics.jax.org/ |
| | Heart Weight | | | TNNI3 | searches/ |
| | | | | | Phat.cgi?id=MP:0002833 |
| 9.35 | Abnormal | 33 | 2 | ACTA1, TNNI3 | http://www.informatics.jax.org/ |
| | Sarcomere | | | | searches/ |
| | Morphology | | | | Phat.cgi?id=MP:0004090 |
| 9.17 | Abnormal | 1 | 1 | TEAD4 | http://www.informatics.jax.org/ |
| | Morula | | | | searches/ |
| | Morphology | | | | Phat.cgi?id=MP:0012058 |
| 9.17 | Abnormal | 1 | 1 | IL4R | http://www.informatics.jax.org/ |
| | Circulating | | | | searches/ |
| | Interferon- | | | | Phat.cgi?id=MP:0008551 |
| | gamma Level | | | | |
| 9.10 | Decreased | 36 | 2 | ACTA1, GYS1 | http://www.informatics.jax.org/ |
| | Glycogen | | | | searches/ |
| | Level | | | | Phat.cgi?id=MP:0005439 |
| 8.29 | Increased | 48 | 2 | MIF, IL4R | http://www.informatics.jax.org/ |
| | Interleukin-4 | | | | searches/ |
| | Secretion | | | | Phat.cgi?i=MP:0008699 |
| 8.29 | Congestive | 48 | 2 | MYL2, TNNI3 | http://www.informatics.jax.org/ |
| | Heart Failure | | | | searches/ |
| | | | | | Phat.cgi?id=MP:0006138 |
| 8.17 | Increased | 2 | 1 | GYS1 | http://www.informatics.jax.org/ |
| | Glycogen | | | | searches/ |
| | Catabolism | | | | Phat.cgi?id=MP:0002714 |
| | Rate | | | | |
| 7.59 | Increased | 3 | 1 | NXPH4 | http://www.informatics.jax.org/ |
| | Urine Nitrite | | | | searches/ |
| | Level | | | | Phat.cgi?id=MP:0011741 |
| 7.18 | Increased | 4 | 1 | IL4R | http://www.informatics.jax.org/ |
| | Respiratory | | | | searches/ |
| | Mucosa | | | | Phat.cgi?id=MP:0010861 |
| | Goblet Cell | | | | |
| | Number | | | | |
| 6.86 | Increased | 5 | 1 | IL4R | http://www.informatics.jax.org/ |
| | Transforming | | | | searches/ |
| | Growth | | | | Phat.cgi?id=MP:0008837 |
| | Factor Level | | | | |
| 6.80 | Increased | 82 | 2 | MIF, IL4R | http://www.informatics.jax.org/ |
| | Susceptibility | | | | searches/ |
| | to Parasitic | | | | Phat.cgi?id=MP:0005027 |
| | Infection | | | | |
| 6.67 | Cardiac | 86 | 2 | MYL2, GYS1 | http://www.informatics.jax.org/ |
| | Fibrosis | | | | searches/ |
| | | | | | Phat.cgi?id=MP:0003141 |
| 6.59 | Decreased T- | 6 | 1 | IL4R | http://www.informatics.jax.org/ |
| | helper 2 Cell | | | | searches/ |
| | Number | | | | Phat.cgi?id=MP:0008091 |
| 6.37 | Decreased | 7 | 1 | MIF | http://www.informatics.jax.org/ |
| | Mitotic Index | | | | searches/ |
| | | | | | Phat.cgi?id=MP:0004759 |
| 6.37 | Abnormal | 7 | 1 | IL4R | http://www.informatics.jax.org/ |
| | Macrophage | | | | searches/ |
| | Activation | | | | Phat.cgi?id=MP:0011075 |
| | Involved in | | | | |
| | Immune | | | | |
| | Response | | | | |
| 6.37 | Abnormal | 7 | 1 | MYL2 | http://www.informatics.jax.org/ |
| | Interventricular | | | | searches/ |
| | Groove | | | | Phat.cgi?id=MP:0004032 |
| | Morphology | | | | |
| 6.18 | Absent | 8 | 1 | TEAD4 | http://www.informatics.jax.org/ |
| | Trophectoderm | | | | searches/ |
| | | | | | Phat.cgi?id=MP:0012102 |

Data File 3. GeneAnalytics characterization of “Innate Immunity
Lobe” of RVF-associated module.
GeneAnalytics-Pathway results
Statistics:

| | | |
|---|---|---|
| # Identified genes: 51 | # Matched genes: 25 | # Matched entities: 20 |

Matching score statistics:

| | | |
|---|---|---|
| # High score matches: 1 | # Med score matches: 19 | # Low score matches: 0 |

Results

| | | | # SuperPath | | |
|---|---|---|---|---|---|
| | SuperPath | # SuperPath | Matched | Matched Genes | |
| Score | Name | Total Genes | Genes | (Symbols) | Evidence URL |
| 23.45 | Innate | 2132 | 20 | ADAM8, IFI30, | http://pathcards. |
| | Immune | | | FPR1, IFITM2, | genecards.org/ |
| | System | | | HLA-G, HLA- | card/innate immune_ |
| | | | | DQA2, CSF3R, | system |
| | | | | SLC11A1, CTSH, | |
| | | | | FGF18, | |
| | | | | HSP90B1, GRN, | |
| | | | | LILRA5, TCIRG1, | |
| | | | | LILRB3, C5AR1, | |
| | | | | PDXK, PLAUR, | |
| | | | | CDA, TREM1 | |
| 11.33 | Immunoregulatory | 135 | 4 | HLA-G, LILRA5, | http://pathcards. |
| | Interactions | | | LILRB3, TREM1 | genecards.org/ |
| | Between A | | | | card/ |
| | Lymphoid | | | | immunoregulatory_ |
| | and A Non- | | | | interactions_ |
| | Lymphoid | | | | between_a_ |
| | Cell | | | | lymphoid_and_ |
| | | | | | a_non- |
| | | | | | lymphoid_cell |
| 11.28 | Staphylococcus | 56 | 3 | FPR1, HLA- | http://pathcards. |
| | Aureus | | | DQA2, C5AR1 | genecards.org/ |
| | Infection | | | | card/staphylococcus_ |
| | | | | | aureus_ |
| | | | | | infection |
| 10.69 | Cytokine | 761 | 8 | IFI30, FPR1, | http://pathcards. |
| | Signaling in | | | IFITM2, HLA-G, | genecards.org/ |
| | Immune | | | HLA-DQA2, | card/cytokine_ |
| | System | | | CSF3R, FGF18, | signaling_in_ |
| | | | | HSP90B1 | immune_system |
| 9.95 | Class I MHC | 823 | 8 | IFI30, HLA-G, | http://pathcards. |
| | Mediated | | | HLA-DQA2, | genecards.org/ |
| | Antigen | | | CTSH, FGF18, | card/class_i_mhc_ |
| | Processing | | | LILRA5, LILRB3, | mediated_ |
| | and | | | TREM1 | antigen_processing_ |
| | Presentation | | | | and_presentation |
| 9.18 | Interferon | 202 | 4 | IFI30, IFITM2, | http://pathcards. |
| | Gamma | | | HLA-G, HLA- | genecards.org/ |
| | Signaling | | | DQA2 | card/interferon_ |
| | | | | | gamma_signaling |
| 8.76 | MHC Class II | 103 | 3 | IFI30, HLA- | http://pathcards. |
| | Antigen | | | DQA2, CTSH | genecards.org/ |
| | Presentation | | | | card/mhc_class_ii_ |
| | | | | | antigen_presentation |
| 8.05 | Lysosome | 123 | 3 | SLC11A1, CTSH, | http://pathcards. |
| | | | | TCIRG1 | genecards.org/ |
| | | | | | card/lysosome |
| 7.89 | Osteoclast | 128 | 3 | LILRA5, LILRB3, | http://pathcards. |
| | Differentiation | | | SPI1 | genecards.org/ |
| | | | | | card/osteoclast_ |
| | | | | | differentiation |
| 7.20 | Phagosome | 152 | 3 | HLA-G, HLA- | http://pathcards. |
| | | | | DQA2, TCIRG1 | genecards.org/ |
| | | | | | card/phagosome |
| 6.81 | Ras | 322 | 4 | FPR1, FGF18, | http://pathcards. |
| | Signaling | | | RGS14, RIN1 | genecards.org/ |
| | Pathway | | | | card/ras_signaling_ |
| | | | | | pathway |
| 6.61 | G-protein | 60 | 2 | IFI30, HLA-DQA2 | http://pathcards. |
| | Signaling N- | | | | genecards.org/ |
| | RAS | | | | card/g- |
| | Regulation | | | | protein_signaling_ |
| | Pathway | | | | n- |
| | | | | | ras_regulation_ |
| | | | | | pathway |
| 6.57 | Validated | 61 | 2 | SLC11A1, SPI1 | http://pathcards. |
| | Targets of C- | | | | genecards.org/ |
| | MYC | | | | card/validated_ |
| | Transcriptional | | | | targets_of_c- |
| | Repression | | | | myc_transcriptional_ |
| | | | | | repression |
| 6.48 | Signaling By | 63 | 2 | FGF18, CUX1 | http://pathcards. |
| | FGFR2 in | | | | genecards.org/ |
| | Disease | | | | card/signaling_by_ |
| | | | | | fgfr2_in_disease |
| 6.32 | Pyrimidine | 5 | 1 | CDA | http://pathcards. |
| | Deoxyribonucleosides | | | | genecards.org/ |
| | Degradation | | | | card/pyrimidine_ |
| | | | | | deoxyribonucleosides_ |
| | | | | | degradation |
| 6.08 | Epstein-Barr | 203 | 3 | HLA-G, HLA- | http://pathcards. |
| | Virus | | | DQA2, SPI1 | genecards.org/ |
| | Infection | | | | card/epstein- |
| | | | | | barr_virus_infection |
| 5.82 | Pathways in | 395 | 4 | CSF3R, FGF18, | http://pathcards. |
| | Cancer | | | HSP90B1, SPI1 | genecards.org/ |
| | | | | | card/pathways_in_ |
| | | | | | cancer |
| 5.76 | Complement | 82 | 2 | C5AR1, PLAUR | http://pathcards. |
| | and | | | | genecards.org/ |
| | Coagulation | | | | card/complement_ |
| | Cascades | | | | and_coagulation_ |
| | | | | | cascades |
| 5.64 | Free Fatty | 8 | 1 | GNA15 | http://pathcards. |
| | Acids | | | | genecards.org/ |
| | Regulate | | | | card/free_fatty_ |
| | Insulin | | | | acids_regulate_ |
| | Secretion | | | | insulin_secretion |
| 5.64 | Vitamin B6 | 8 | 1 | PDXK | http://pathcards. |
| | Metabolism | | | | genecards.org/ |
| | | | | | card/vitamin_b6_ |
| | | | | | metabolism |

GeneAnalytics-Function-based Analysis, GO-Biological Process results
Statistics:

| | | |
|---|---|---|
| # Identified genes: 51 | # Matched genes: 29 | # Matched entities: 20 |

Matching score statistics:

| | | |
|---|---|---|
| # High score matches: 2 | # Med score matches: 18 | # Low score matches: 0 |

Results

| | | | # Matched | Matched Genes | |
|---|---|---|---|---|---|
| Score | Name | # Genes | Genes | (Symbols) | Evidence URL |
| 25.02 | Neutrophil | 484 | 11 | ADAM8, FPR1, | http://amigo. |
| | Degranulation | | | SLC11A1, CTSH, | geneontology.org/ |
| | | | | GRN, TCIRG1, | amigo/term/GO: |
| | | | | LILRB3, C5AR1, | 0043312 |
| | | | | PDXK, PLAUR, | |
| | | | | CDA | |
| 13.65 | Negative | 5 | 2 | RGS14, RIN1 | http://amigo. |
| | Regulation of | | | | geneontology.org/ |
| | Synaptic | | | | amigo/term/GO: |
| | Plasticity | | | | 0031914 |
| 11.39 | Complement | 11 | 2 | FPR1, C5AR1 | http://amigo. |
| | Receptor | | | | geneontology.org/ |
| | Mediated | | | | amigo/term/GO: |
| | Signaling | | | | 0002430 |
| | Pathway | | | | |
| 11.36 | Defense | 55 | 3 | SSC5D, | http://amigo. |
| | Response to | | | SLC11A1, | geneontology.org/ |
| | Gram- | | | HIST1H2BK | amigo/term/GO: |
| | negative | | | | 0050829 |
| | Bacterium | | | | |
| 11.15 | Vacuolar | 12 | 2 | SLC11A1, | http://amigo. |
| | Acidification | | | TCIRG1 | geneontology.org/ |
| | | | | | amigo/term/GO: |
| | | | | | 0007035 |
| 10.92 | Cellular | 61 | 3 | HLA-G, TCIRG1, | http://amigo. |
| | Defense | | | C5AR1 | geneontology.org/ |
| | Response | | | | amigo/term/GO: |
| | | | | | 0006968 |
| 10.92 | Phospholipase | 13 | 2 | GNA15, | http://amigo. |
| | C-activating | | | SLC9A3R1 | geneontology.org/ |
| | Dopamine | | | | amigo/term/GO: |
| | Receptor | | | | 0060158 |
| | Signaling | | | | |
| | Pathway | | | | |
| 10.59 | Neutrophil | 66 | 3 | CSF3R, C5AR1, | http://amigo. |
| | Chemotaxis | | | TREM1 | geneontology.org/ |
| | | | | | amigo/term/GO: |
| | | | | | 0030593 |
| 10.59 | Cell | 66 | 3 | FPR1, CXCL14, | http://amigo. |
| | Chemotaxis | | | C5AR1 | geneontology.org/ |
| | | | | | amigo/term/GO: |
| | | | | | 0060326 |
| 10.48 | Chemotaxis | 158 | 4 | FPR1, CXCL14, | http://amigo. |
| | | | | CSAR1, PLAUR | geneontology.org/ |
| | | | | | amigo/term/GO: |
| | | | | | 0006935 |
| 10.47 | Phospholipase | 68 | 3 | FPR1, GNA15, | http://amigo. |
| | C-activating | | | CSAR1 | geneontology.org/ |
| | G-protein | | | | amigo/term/GO: |
| | Coupled | | | | 0007200 |
| | Receptor | | | | |
| | Signaling | | | | |
| | Pathway | | | | |
| 10.29 | Interferon- | 71 | 3 | IFI30, HLA-G, | http://amigo. |
| | gamma- | | | HLA-DQA2 | geneontology.org/ |
| | mediated | | | | amigo/term/GO: |
| | Signaling | | | | 0060333 |
| | Pathway | | | | |
| 10.29 | Defense | 71 | 3 | SSC5D, | http://amigo. |
| | Response to | | | HIST1H2BK, | geneontology.org/ |
| | Gram- | | | C5AR1 | amigo/term/GO: |
| | positive | | | | 0050830 |
| | Bacterium | | | | |
| 10.15 | Microvillus | 17 | 2 | SLC9A3R1, | http://amigo. |
| | Assembly | | | FXYD5 | geneontology.org/ |
| | | | | | amigo/term/GO: |
| | | | | | 0030033 |
| 10.15 | Chondrocyte | 17 | 2 | SFRP2, FGF18 | http://amigo. |
| | Development | | | | geneontology.org/ |
| | | | | | amigo/term/GO: |
| | | | | | 0002063 |
| 9.69 | Defense | 20 | 2 | SLC11A1, | http://amigo. |
| | Response to | | | CCDC88B | geneontology.org/ |
| | Protozoan | | | | amigo/term/GO: |
| | | | | | 0042832 |
| 9.36 | Immune | 495 | 6 | IFITM2, HLA- | http://amigo. |
| | Response | | | DQA2, SLC11A1, | geneontology.org/ |
| | | | | CST7, CXCL14, | amigo/term/GO: |
| | | | | C5AR1 | 0006955 |
| 9.06 | Response to | 25 | 2 | IFITM2, SLC11A1 | http://amigo. |
| | Interferon- | | | | geneontology.org/ |
| | gamma | | | | amigo/term/GO: |
| | | | | | 0034341 |
| 8.95 | Activation of | 26 | 2 | GNA15, C5AR1 | http://amigo. |
| | Phospholipase | | | | geneontology.org/ |
| | C Activity | | | | amigo/term/GO: |
| | | | | | 0007202 |
| 8.74 | Positive | 28 | 2 | SLC11A1, | http://amigo. |
| | Regulation of | | | CCDC88B | geneontology.org/ |
| | Cytokine | | | | amigo/term/GO: |
| | Production | | | | 0001819 |

GeneAnalytics-Function-based Analysis, GO-Molecular Process results
Statistics:

| | | |
|---|---|---|
| # Identified genes: 51 | # Matched genes: 19 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 11.97 | Complement | 9 | 2 | FPR1, C5AR1 | http://amigo. |
| | Receptor | | | | geneontology.org/ |
| | Activity | | | | amigo/term/GO: |
| | | | | | 0004875 |
| 10.70 | GTPase | 14 | 2 | RGS14, FMNL1 | http://amigo. |
| | Activating | | | | geneontology.org/ |
| | Protein | | | | amigo/term/GO: |
| | Binding | | | | 0032794 |
| 8.84 | Fibronectin | 27 | 2 | SSC5D, SFRP2 | http://amigo. |
| | Binding | | | | geneontology.org/ |
| | | | | | amigo/term/GO: |
| | | | | | 0001968 |
| 8.63 | Granulocyte | 1 | 1 | CSF3R | http://amigo. |
| | Colony- | | | | geneontology.org/ |
| | stimulating | | | | amigo/term/GO: |
| | Factor | | | | 0051916 |
| | Binding | | | | |
| 8.63 | HLA-A | 1 | 1 | CTSH | http://amigo. |
| | Specific | | | | geneontology.org/ |
| | Activating | | | | amigo/term/GO: |
| | MHC Class I | | | | 0030108 |
| | Receptor | | | | |
| | Activity | | | | |
| 8.63 | Complement | 1 | 1 | C5AR1 | http://amigo. |
| | Component | | | | geneontology.org/ |
| | C5a Binding | | | | amigo/term/GO: |
| | | | | | 0001856 |
| 8.63 | Metal Ion: | 1 | 1 | SLC11A1 | http://amigo. |
| | proton | | | | geneontology.org/ |
| | Antiporter | | | | amigo/term/GO: |
| | Activity | | | | 0051139 |
| 8.63 | RNA | 1 | 1 | PRDX5 | http://amigo. |
| | Polymerase | | | | geneontology.org/ |
| | III Regulatory | | | | amigo/term/GO: |
| | Region DNA | | | | 0001016 |
| | Binding | | | | |
| 8.63 | Urokinase | 1 | 1 | PLAUR | http://amigo. |
| | Plasminogen | | | | geneontology.org/ |
| | Activator | | | | amigo/term/GO: |
| | Receptor | | | | 0030377 |
| | Activity | | | | |
| 8.63 | Pyridoxal | 1 | 1 | PDXK | http://amigo. |
| | Kinase | | | | geneontology.org/ |
| | Activity | | | | amigo/term/GO: |
| | | | | | 0008478 |
| 8.60 | Receptor | 226 | 4 | CSF3R, LILRB3, | http://amigo. |
| | Activity | | | PLAUR, TREM1 | geneontology.org/ |
| | | | | | amigo/term/GO: |
| | | | | | 0004872 |
| 7.63 | Transition | 2 | 1 | SLC11A1 | http://amigo. |
| | Metal Ion | | | | geneontology.org/ |
| | Transmembrane | | | | amigo/term/GO: |
| | Transporter | | | | 0046915 |
| | Activity | | | | |
| 7.63 | Lithium Ion | 2 | 1 | PDXK | http://amigo. |
| | Binding | | | | geneontology.org/ |
| | | | | | amigo/term/GO: |
| | | | | | 0031403 |
| 7.63 | Oxidoreductase | 2 | 1 | IFI30 | http://amigo. |
| | Activity, | | | | geneontology.org/ |
| | Acting on A | | | | amigo/term/GO: |
| | Sulfur Group | | | | 0016667 |
| | of Donors | | | | |
| 7.63 | Complement | 2 | 1 | C5AR1 | http://amigo. |
| | Component | | | | geneontology.org/ |
| | C5a | | | | amigo/term/GO: |
| | Receptor | | | | 0004878 |
| | Activity | | | | |
| 7.63 | Dopamine | 2 | 1 | SLC9A3R1 | http://amigo. |
| | Receptor | | | | geneontology.org/ |
| | Binding | | | | amigo/term/GO: |
| | | | | | 0050780 |
| 7.05 | AMP | 3 | 1 | AMPD2 | http://amigo. |
| | Deaminase | | | | geneontology.org/ |
| | Activity | | | | amigo/term/GO: |
| | | | | | 0003876 |
| 7.05 | Type 2 | 3 | 1 | FGF18 | http://amigo. |
| | Fibroblast | | | | geneontology.org/ |
| | Growth | | | | amigo/term/GO: |
| | Factor | | | | 0005111 |
| | Receptor | | | | |
| 6.85 | Binding | 55 | 2 | ADAM8, | http://amigo. |
| | Protein Self- | | | SLC9A3R1 | geneontology.org/ |
| | association | | | | amigo/term/GO: |
| | | | | | 0043621 |
| 6.64 | Myosin II | 4 | 1 | SLC9A3R1 | http://amigo. |
| | Binding | | | | geneontology.org/ |
| | | | | | amigo/term/GO: |
| | | | | | 0045159 |

GeneAnalytics-Function-based Analysis, Phenotype results
Statistics:

| | | |
|---|---|---|
| # Identified genes: 51 | # Matched genes: 18 | # Matched entities: 20 |

Matching score statistics:

| | | |
|---|---|---|
| # High score matches: 1 | # Med score matches: 19 | # Low score matches: 0 |

Results

| | | | # Matched | Matched Genes | |
|---|---|---|---|---|---|
| Score | Name | # Genes | Genes | (Symbols) | Evidence URL |
| 18.60 | Increased | 241 | 7 | FPR1, HLA-G, | http://www. |
| | Susceptibility | | | SLC11A1, CUX1, | informatics.jax.org/ |
| | to Bacterial | | | GRN, C5AR1, | searches/Phat. |
| | Infection | | | PLAUR | cgi?id=MP:0002412 |
| 13.13 | Abnormal | 6 | 2 | CSF3R, SPI1 | http://www.informatics. |
| | Monocyte | | | | jax.org/ |
| | Morphology | | | | searches/Phat. |
| | | | | | cgi?id=MP:0002620 |
| 11.97 | Abnormal | 9 | 2 | CSF3R, SPI1 | http://www. |
| | Neutrophil | | | | informatics.jax.org/ |
| | Differentiation | | | | searches/Phat. |
| | | | | | cgi?id=MP:0002415 |
| 11.15 | Increased B- | 12 | 2 | LILRB3, SPI1 | http://www. |
| | 1 B Cell | | | | informatics.jax.org/ |
| | Number | | | | searches/Phat. |
| | | | | | cgi?id=MP:0004977 |
| 10.32 | Failure of | 16 | 2 | TCIRG1, SPI1 | http://www. |
| | Tooth | | | | informatics.jax.org/ |
| | Eruption | | | | searches/Phat. |
| | | | | | cgi?id=MP:0000121 |
| 9.85 | Decreased | 79 | 3 | CSF3R, SPI1, | http://www.informatics. |
| | Neutrophil | | | TREM1 | jax.org/ |
| | Cell Number | | | | searches/Phat. |
| | | | | | cgi?id=MP:0000222 |
| 9.84 | Abnormal | 19 | 2 | FPR1, HLA-G | http://www.informatics. |
| | Innate | | | | jax.org/ |
| | Immunity | | | | searches/Phat. |
| | | | | | cgi?id=MP:0002419 |
| 9.73 | Abnormal | 182 | 4 | HPS1, GRN, | http://www.informatics. |
| | Macrophage | | | TCIRG1, PLAUR | jax.org/ |
| | Physiology | | | | searches/Phat. |
| | | | | | cgi?id=MP:0002451 |
| 9.55 | Increased | 21 | 2 | TCIRG1, C5AR1 | http://www.informatics. |
| | Spleen | | | | jax.org/ |
| | Germinal | | | | searches/Phat. |
| | Center | | | | cgi?id=MP:0008481 |
| | Number | | | | |
| 9.42 | Increased | 22 | 2 | FGF18, TCIRG1 | http://www. |
| | Long Bone | | | | informatics.jax.org/ |
| | Epiphyseal | | | | searches/Phat. |
| | Plate Size | | | | cgi?id=MP:0006398 |
| 9.34 | Decreased B | 196 | 4 | IGLL5, CUX1, | http://www. |
| | Cell Number | | | TCIRG1, SPI1 | informatics.jax.org/ |
| | | | | | searches/Phat. |
| | | | | | cgi?id=MP:0005017 |
| 9.31 | Abnormal | 90 | 3 | CSF3R, C5AR1, | http://www. |
| | Neutrophil | | | PLAUR | informatics.jax.org/ |
| | Physiology | | | | searches/Phat. |
| | | | | | cgi?id=MP:0002463 |
| 9.06 | Decreased | 25 | 2 | CSF3R, SPI1 | http://www. |
| | Granulocyte | | | | informatics.jax.org/ |
| | Number | | | | searches/Phat. |
| | | | | | cgi?id=MP:0000334 |
| 9.06 | Abnormal | 25 | 2 | SEMA5B, GRN | http://www. |
| | Neurite | | | | informatics.jax.org/ |
| | Morphology | | | | searches/Phat. |
| | | | | | cgi?id=MP:0008415 |
| 8.64 | Increased | 29 | 2 | FGF18, TCIRG1 | http://www. |
| | Width of | | | | informatics.jax.org/ |
| | Hypertrophic | | | | searches/Phat. |
| | Chondrocyte | | | | cgi?id=MP:0003408 |
| | Zone | | | | |
| 8.63 | Absent Awl | 1 | 1 | CUX1 | http://www. |
| | Hair | | | | informatics.jax.org/ |
| | | | | | searches/Phat. |
| | | | | | cgi?id=MP:0006364 |
| 8.45 | Osteopetrosis | 31 | 2 | TCIRG1, SPI1 | http://www. |
| | | | | | informatics.jax.org/ |
| | | | | | searches/Phat. |
| | | | | | cgi?id=MP:0000067 |
| 8.11 | Short Radius | 35 | 2 | SFRP2, FGF18 | http://www. |
| | | | | | informatics.jax.org/ |
| | | | | | searches/Phat. |
| | | | | | cgi?id=MP:0004355 |
| 8.03 | Decreased | 36 | 2 | CSF3R, TREM1 | http://www. |
| | Monocyte | | | | informatics.jax.org/ |
| | Cell Number | | | | searches/Phat. |
| | | | | | cgi?id=MP:0000223 |
| 7.95 | Increased | 37 | 2 | FKBP11, TCIRG1 | http://www. |
| | Bone Mineral | | | | informatics.jax.org/ |
| | Content | | | | searches/Phat. |
| | | | | | cgi?id=MP:0010123 |

Data File 4. GeneAnalytics characterization of “Metabolism and
Intracellular Signaling Lobe” of RVFassociated module.
GeneAnalytics-Pathway results
Statistics:

| | | |
|---|---|---|
| # Identified genes: 60 | # Matched genes: 28 | # Matched entities: 20 |

Matching score statistics:

| | | |
|---|---|---|
| # High score matches: 0 | # Med score matches: 20 | # Low score matches: 0 |

Results

| | | | # SuperPath | | |
|---|---|---|---|---|---|
| | SuperPath | # SuperPath | Matched | Matched Genes | |
| Score | Name | Total Genes | Genes | (Symbols) | Evidence URL |
| 11.50 | NOTCH2 | 45 | 3 | DTX2, UBB, | http://pathcards. |
| | Activation | | | DLL4 | genecards.org/ |
| | and | | | | card/notch2_ |
| | Transmission | | | | activation_and_ |
| | of Signal to | | | | transmission_of_signal_ |
| | The Nucleus | | | | to_the_nucleus |
| 11.43 | FMLP | 317 | 6 | UBB, PSMB10, | http://pathcards. |
| | Pathway | | | GAPDH, GNB2, | genecards.org/ |
| | | | | DCTN1, GRINA | card/fmlp_pathway |
| 9.86 | Cori Cycle | 16 | 2 | GAPDH, PFKP | http://pathcards. |
| | | | | | genecards.org/ |
| | | | | | card/cori_cycle |
| 8.88 | Cytoskeletal | 304 | 5 | DCTN1, MYL9, | http://pathcards. |
| | Signaling | | | AP2M1, MYH9, | genecards.org/ |
| | | | | TPM1 | card/cytoskeletal_ |
| | | | | | signaling |
| 8.08 | Surfactant | 30 | 2 | ABCA3, ADRA2C | http://pathcards. |
| | Metabolism | | | | genecards.org/ |
| | | | | | card/surfactant_ |
| | | | | | metabolism |
| 7.72 | Signaling By | 113 | 3 | DTX2, UBB, | http://pathcards. |
| | NOTCH1 | | | DLL4 | genecards.org/ |
| | | | | | card/signaling_by_ |
| | | | | | notch1 |
| 7.57 | Smooth | 36 | 2 | MYL9, TPM1 | http://pathcards. |
| | Muscle | | | | genecards.org/ |
| | Contraction | | | | card/smooth_ |
| | | | | | muscle_contraction |
| 7.42 | Integrin | 38 | 2 | ADRA2C, VWF | http://pathcards. |
| | AlphaIIb | | | | genecards.org/ |
| | Beta3 | | | | card/integrin_ |
| | Signaling | | | | alphaiib_beta3_ |
| | | | | | signaling |
| 7.29 | Glucose | 126 | 3 | UBB, GAPDH, | http://pathcards. |
| | Metabolism | | | PFKP | genecards.org/ |
| | | | | | card/glucose_ |
| | | | | | metabolism |
| 7.28 | Proteolysis | 40 | 2 | UBB, PML | http://pathcards. |
| | Putative | | | | genecards.org/ |
| | SUMO-1 | | | | card/proteolysis_ |
| | Pathway | | | | putative_sumo- |
| | | | | | 1_pathway |
| 7.21 | Striated | 41 | 2 | MYL9, TPM1 | http://pathcards. |
| | Muscle | | | | genecards.org/ |
| | Contraction | | | | card/striated_ |
| | | | | | muscle_contraction |
| 6.91 | Signaling By | 139 | 3 | UBB, PSMB10, | http://pathcards. |
| | Hedgehog | | | P4HB | genecards.org/ |
| | | | | | card/signaling_by_ |
| | | | | | hedgehog |
| 6.82 | RNAi | 3 | 1 | TARBP2 | http://pathcards. |
| | Pathway | | | | genecards.org/ |
| | | | | | card/mai_pathway |
| 6.74 | EPH-Ephrin | 145 | 3 | MYL9, AP2M1, | http://pathcards. |
| | Signaling | | | MYH9 | genecards.org/ |
| | | | | | card/eph- |
| | | | | | ephrin_signaling |
| 6.72 | Immune | 146 | 3 | GNB2, MYL9, | http://pathcards. |
| | Response | | | MYH9 | genecards.org/ |
| | CCR3 | | | | card/immune_ |
| | Signaling in | | | | response_ccr3_ |
| | Eosinophils | | | | signaling_in_ |
| | | | | | eosinophils |
| 6.56 | AMPK | 152 | 3 | ADRA2C, GNB2, | http://pathcards. |
| | Enzyme | | | PFKP | genecards.org/ |
| | Complex | | | | card/ampk_ |
| | Pathway | | | | enzyme_complex_ |
| | | | | | pathway |
| 6.22 | Protein | 166 | 3 | RRBP1, P4HB, | http://pathcards. |
| | Processing in | | | HYOU1 | genecards.org/ |
| | Endoplasmic | | | | card/protein_ |
| | Reticulum | | | | processing_in_ |
| | | | | | endoplasmic_reticulum |
| 6.02 | Metabolism | 2543 | 14 | MBOAT7, | http://pathcards. |
| | | | | FADS2, AGPAT2, | genecards.org/ |
| | | | | ADRA2C, UBB, | card/metabolism |
| | | | | PSMB10, | |
| | | | | GAPDH, GNB2, | |
| | | | | AP2M1, P4HB, | |
| | | | | GUK1, HYI, | |
| | | | | PFKP, PIGT | |
| 5.89 | Wnt/ | 181 | 3 | DTX2, GDF15, | http://pathcards. |
| | Hedgehog/ | | | DLL4 | genecards.org/ |
| | Notch | | | | card/wnt_hedge |
| | | | | | hog_notch |
| 5.87 | ADP | 67 | 2 | UBB, GNB2 | http://pathcards. |
| | Signalling | | | | genecards.org/ |
| | Through P2Y | | | | card/adp_signalling_ |
| | Purinoceptor | | | | through_p2y_ |
| | 1 | | | | purinoceptor_1 |

GeneAnalytics-Function-based Analysis, GO-Biological Process results
Statistics:

| | | |
|---|---|---|
| # Identified genes: 60 | # Matched genes: 18 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 10.37 | Negative | 59 | 3 | UBB, MIIP, | http://amigo. |
| | Regulation of | | | PSMB10 | geneontology.org/ |
| | G2/M | | | | amigo/term/GO: |
| | Transition of | | | | 0010972 |
| | Mitotic Cell | | | | |
| | Cycle | | | | |
| 10.04 | Endoplasmic | 15 | 2 | GRINA, PML | http://amigo. |
| | Reticulum | | | | geneontology.org/ |
| | Calcium Ion | | | | amigo/term/GO: |
| | Homeostasis | | | | 0032469 |
| 9.69 | Regulation of | 17 | 2 | IRF7, UBB | http://amigo. |
| | Type I | | | | geneontology.org/ |
| | Interferon | | | | amigo/term/GO: |
| | Production | | | | 0032479 |
| 9.52 | Regulation of | 18 | 2 | MYL9, TPM1 | http://amigo. |
| | Muscle | | | | geneontology.org/ |
| | Contraction | | | | amigo/term/GO: |
| | | | | | 0006937 |
| 9.17 | Response to | 79 | 3 | SDF2L1, P4HB, | http://amigo. |
| | Endoplasmic | | | HYOU1 | geneontology.org/ |
| | Reticulum | | | | amigo/term/GO: |
| | Stress | | | | 0034976 |
| 8.51 | Transforming | 93 | 3 | UBB, GDF15, | http://amigo. |
| | Growth | | | PML | geneontology.org/ |
| | Factor Beta | | | | amigo/term/GO: |
| | Receptor | | | | 0007179 |
| | Signaling | | | | |
| | Pathway | | | | |
| 8.40 | Negative | 1 | 1 | MYH9 | http://amigo. |
| | Regulation of | | | | geneontology.org/ |
| | Actin | | | | amigo/term/GO: |
| | Filament | | | | 1903919 |
| | Severing | | | | |
| 8.40 | Regulation of | 1 | 1 | PML | http://amigo. |
| | Calcium Ion | | | | geneontology.org/ |
| | Transport | | | | amigo/term/GO: |
| | Into Cytosol | | | | 0010522 |
| 8.40 | Hypothalamus | 1 | 1 | UBB | http://amigo. |
| | Gonadotrophin- | | | | geneontology.org/ |
| | releasing | | | | amigo/term/GO: |
| | Hormone | | | | 0021888 |
| | Neuron | | | | |
| | Development | | | | |
| 8.40 | Positive | 1 | 1 | TPM1 | http://amigo. |
| | Regulation of | | | | geneontology.org/ |
| | Heart Rate | | | | amigo/term/GO: |
| | By | | | | 0003065 |
| | Epinephrine | | | | |
| 8.40 | Regulation of | 1 | 1 | TARBP2 | http://amigo. |
| | Viral | | | | geneontology.org/ |
| | Transcription | | | | amigo/term/GO: |
| | | | | | 0046782 |
| 8.40 | Positive | 1 | 1 | GAPDH | http://amigo. |
| | Regulation | | | | geneontology.org/ |
| | By Organism | | | | amigo/term/GO: |
| | of Apoptotic | | | | 0052501 |
| | Process in | | | | |
| | Other | | | | |
| | Organism | | | | |
| | Involved in | | | | |
| | Symbiotic | | | | |
| | Interaction | | | | |
| 8.40 | DGDP | 1 | 1 | GUK1 | http://amigo. |
| | Biosynthetic | | | | geneontology.org/ |
| | Process | | | | amigo/term/GO: |
| | | | | | 0006185 |
| 8.40 | GDP | 1 | 1 | GUK1 | http://amigo. |
| | Biosynthetic | | | | geneontology.org/ |
| | Process | | | | amigo/term/GO: |
| | | | | | 0046711 |
| 8.40 | DATP | 1 | 1 | GUK1 | http://amigo. |
| | Metabolic | | | | geneontology.org/ |
| | Process | | | | amigo/term/GO: |
| | | | | | 0046060 |
| 8.40 | DGMP | 1 | 1 | GUK1 | http://amigo. |
| | Metabolic | | | | geneontology.org/ |
| | Process | | | | amigo/term/GO: |
| | | | | | 0046054 |
| 8.40 | Regulation of | 1 | 1 | NECAB3 | http://amigo. |
| | Amyloid | | | | geneontology.org/ |
| | Precursor | | | | amigo/term/GO: |
| | Protein | | | | 0042984 |
| | Biosynthetic | | | | |
| | Process | | | | |
| 8.40 | Cellular | 1 | 1 | SBNO2 | http://amigo. |
| | Response to | | | | geneontology.org/ |
| | Leukemia | | | | amigo/term/GO: |
| | Inhibitory | | | | 1990830 |
| | Factor | | | | |
| 8.40 | Establishment | 1 | 1 | IRF7 | http://amigo. |
| | of Viral | | | | geneontology.org/ |
| | Latency | | | | amigo/term/GO: |
| | | | | | 0019043 |
| 8.40 | Regulation of | 1 | 1 | IRF7 | http://amigo. |
| | MyD88- | | | | geneontology.org/ |
| | independent | | | | amigo/term/GO: |
| | Toll-like | | | | 0034127 |
| | Receptor | | | | |
| | Signaling | | | | |
| | Pathway | | | | |

GeneAnalytics-Function-based Analysis, GO-Molecular Process results
Statistics:

| | | |
|---|---|---|
| # Identified genes: 60 | # Matched genes: 45 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 9.37 | 1- | 19 | 2 | MBOAT7, | http://amigo. |
| | acylglycerol- | | | AGPAT2 | geneontology.org/ |
| | 3-phosphate | | | | amigo/term/GO: |
| | O- | | | | 0003841 |
| | acyltransferase | | | | |
| | Activity | | | | |
| 8.73 | Chaperone | 88 | 3 | SDF2L1, VWF, | http://amigo. |
| | Binding | | | HYOU1 | geneontology.org/ |
| | | | | | amigo/term/GO: |
| | | | | | 0051087 |
| 8.40 | Linoleoyl- | 1 | 1 | FADS2 | http://amigo. |
| | CoA | | | | geneontology.org/ |
| | Desaturase | | | | amigo/term/GO: |
| | Activity | | | | 0016213 |
| 8.40 | Hydroxypyruvate | 1 | 1 | HYI | http://amigo. |
| | Isomerase | | | | geneontology.org/ |
| | Activity | | | | amigo/term/GO: |
| | | | | | 0008903 |
| 8.40 | Peptidyl- | 1 | 1 | GAPDH | http://amigo. |
| | cysteine S- | | | | geneontology.org/ |
| | nitrosylase | | | | amigo/term/GO: |
| | Activity | | | | 0035605 |
| 7.41 | Protein | 9013 | 37 | IRF7, DTX2, | http://amigo. |
| | Binding | | | MBOAT7, | geneontology.org/ |
| | | | | TSEN54, | amigo/term/GO: |
| | | | | ADRA2C, UBB, | 0005515 |
| | | | | MIIP, EIF3F, | |
| | | | | PSMB10, | |
| | | | | HOMER3, | |
| | | | | SH3GLB2, | |
| | | | | GDF15, CRELD2, | |
| | | | | SHPRH, GAPDH, | |
| | | | | VWF, GNB2, | |
| | | | | SRA1, DCTN1, | |
| | | | | NECAB3, | |
| | | | | AP2M1, | |
| | | | | DGCR14, P4HB, | |
| | | | | SWI5, DLL4, | |
| | | | | TBC1D22A, | |
| | | | | LENG1, DRG2, | |
| | | | | TARBP2, MYH9, | |
| | | | | INCA1, HYI, | |
| | | | | C7orf50, PML, | |
| | | | | TPM1, PIGT, | |
| | | | | CCDC85B | |
| 7.40 | Signal | 2 | 1 | AP2M1 | http://amigo. |
| | Sequence | | | | geneontology.org/ |
| | Binding | | | | amigo/term/GO: |
| | | | | | 0005048 |
| 7.40 | Lysophospho | 2 | 1 | MBOAT7 | http://amigo. |
| | lipid | | | | geneontology.org/ |
| | Acyltransferase | | | | amigo/term/GO: |
| | Activity | | | | 0071617 |
| 7.40 | Glyceraldehyde- | 2 | 1 | GAPDH | http://amigo. |
| | 3- | | | | geneontology.org/ |
| | phosphate | | | | amigo/term/GO: |
| | Dehydrogenase | | | | 0004365 |
| | (NAD+) | | | | |
| | (phosphorylating) | | | | |
| | Activity | | | | |
| 7.14 | Protein | 758 | 7 | ADRA2C, VWF, | http://amigo. |
| | Homodimerization | | | GNPTG, | geneontology.org/ |
| | Activity | | | TBC1D22A, | amigo/term/GO: |
| | | | | TARBP2, MYH9, | 0042803 |
| | | | | PML | |
| 6.89 | Structural | 46 | 2 | MYL9, TPM1 | http://amigo. |
| | Constituent | | | | geneontology.org/ |
| | of Muscle | | | | amigo/term/GO: |
| | | | | | 0008307 |
| 6.82 | Alpha2- | 3 | 1 | ADRA2C | http://amigo. |
| | adrenergic | | | | geneontology.org/ |
| | Receptor | | | | amigo/term/GO: |
| | Activity | | | | 0004938 |
| 6.82 | 6- | 3 | 1 | PFKP | http://amigo. |
| | phosphofructokinase | | | | geneontology.org/ |
| | Activity | | | | amigo/term/GO: |
| | | | | | 0003872 |
| 6.82 | Aspartic-type | 3 | 1 | GAPDH | http://amigo. |
| | Endopeptidase | | | | geneontology.org/ |
| | Inhibitor | | | | amigo/term/GO: |
| | Activity | | | | 0019828 |
| 6.82 | Stearoyl-CoA | 3 | 1 | FADS2 | http://amigo. |
| | 9-desaturase | | | | geneontology.org/ |
| | Activity | | | | amigo/term/GO: |
| | | | | | 0004768 |
| 6.82 | UDP-N- | 3 | 1 | GNPTG | http://amigo. |
| | acetylglucosamine- | | | | geneontology.org/ |
| | lysosomal- | | | | amigo/term/GO: |
| | enzyme N- | | | | 0003976 |
| | acetyl- | | | | |
| | glucosaminephospho | | | | |
| | transferase | | | | |
| | Activity | | | | |
| 6.82 | Immunoglobulin | 3 | 1 | VWF | http://amigo. |
| | Binding | | | | geneontology.org/ |
| | | | | | amigo/term/GO: |
| | | | | | 0019865 |
| 6.72 | SMAD | 49 | 2 | ZC3H3, PML | http://amigo. |
| | Binding | | | | geneontology.org/ |
| | | | | | amigo/term/GO: |
| | | | | | 0046332 |
| 6.40 | Pre-miRNA | 4 | 1 | TARBP2 | http://amigo. |
| | Binding | | | | geneontology.org/ |
| | | | | | amigo/term/GO: |
| | | | | | 0070883 |
| 6.40 | Procollagen- | 4 | 1 | P4HB | http://amigo. |
| | proline 4- | | | | geneontology.org/ |
| | dioxygenase | | | | amigo/term/GO: |
| | Activity | | | | 0004656 |

GeneAnalytics-Function-based Analysis, Phenotype results
Statistics:

| | | |
|---|---|---|
| # Identified genes: 60 | # Matched genes: 12 | # Matched entities: 20 |

Matching score statistics:

| | | |
|---|---|---|
| # High score matches: 0 | # Med score matches: 20 | # Low score matches: 0 |

Results

| | | | # Matched | Matched Genes | |
|---|---|---|---|---|---|
| Score | Name | # Genes | Genes | (Symbols) | Evidence URL |
| 9.97 | Abnormal | 65 | 3 | FADS2, VWF, | http://www. |
| | Blood | | | MYH9 | informatics.jax.org/ |
| | Coagulation | | | | searches/Phat. |
| | | | | | cgi?id=MP:0002551 |
| 9.73 | Increased | 69 | 3 | FADS2, VWF, | http://www. |
| | Bleeding | | | MYH9 | informatics.jax.org/ |
| | Time | | | | searches/Phat. |
| | | | | | cgi?id=MP:0005606 |
| 8.96 | Abnormal | 22 | 2 | DLL4, MYH9 | http://www. |
| | Vascular | | | | informatics.jax.org/ |
| | Branching | | | | searches/Phat. |
| | Morphogenesis | | | | cgi?id=MP:0003227 |
| 8.40 | Abnormal L4 | 1 | 1 | GDF15 | http://www. |
| | Dorsal Root | | | | informatics.jax.org/ |
| | Ganglion | | | | searches/Phat. |
| | Morphology | | | | cgi?id=MP:0001019 |
| 8.40 | Absent | 1 | 1 | FADS2 | http://www. |
| | Theca | | | | informatics.jax.org/ |
| | Folliculi | | | | searches/Phat. |
| | | | | | cgi?id=MP:0009368 |
| 8.40 | Decreased | 1 | 1 | GAPDH | http://www. |
| | Glyceraldehyde- | | | | informatics.jax.org/ |
| | 3- | | | | searches/Phat. |
| | phosphate | | | | cgi?id=MP:0011609 |
| | Dehydrogenase | | | | |
| | (NAD+) | | | | |
| | (phosphorylating) | | | | |
| | Activity | | | | |
| 7.90 | Decreased | 32 | 2 | AGPAT2, SRA1 | http://www. |
| | White Fat | | | | informatics.jax.org/ |
| | Cell Size | | | | searches/Phat. |
| | | | | | cgi?id=MP:0009133 |
| 7.80 | Abnormal | 111 | 3 | RASIP1, DLL4, | http://www. |
| | Vitelline | | | MYH9 | informatics.jax.org/ |
| | Vasculature | | | | searches/Phat. |
| | Morphology | | | | cgi?id=MP:0003229 |
| 7.40 | Absent Type | 2 | 1 | ABCA3 | http://www. |
| | I | | | | informatics.jax.org/ |
| | Pneumocytes | | | | searches/Phat. |
| | | | | | cgi?id=MP:0010817 |
| 7.35 | Increased | 39 | 2 | MYH9, INCA1 | http://www. |
| | Megakaryocyte | | | | informatics.jax.org/ |
| | Cell | | | | searches/Phat. |
| | Number | | | | cgi?id=MP:0008254 |
| 7.35 | Abnormal | 39 | 2 | FADS2, INCA1 | http://www. |
| | Spleen White | | | | informatics.jax.org/ |
| | Pulp | | | | searches/Phat. |
| | Morphology | | | | cgi?id=MP:0002357 |
| 7.17 | Abnormal | 130 | 3 | ABCA3, | http://www. |
| | Lipid | | | MBOAT7, | informatics.jax.org/ |
| | Homeostasis | | | AGPAT2 | searches/Phat. |
| | | | | | cgi?id=MP:0002118 |
| 7.02 | Complete | 135 | 3 | GAPDH, DLL4, | http://www. |
| | Preweaning | | | MYH9 | informatics.jax.org/ |
| | Lethality | | | | searches/Phat. |
| | | | | | cgi?id=MP:0011100 |
| 7.02 | Decreased | 44 | 2 | AGPAT2, SRA1 | http://www. |
| | Brown | | | | informatics.jax.org/ |
| | Adipose | | | | searches/Phat. |
| | Tissue | | | | cgi?id=MP:0001780 |
| | Amount | | | | |
| 6.89 | Increased | 46 | 2 | AGPAT2, GDF15 | http://www. |
| | Kidney | | | | informatics.jax.org/ |
| | Weight | | | | searches/Phat. |
| | | | | | cgi?id=MP:0003917 |
| 6.82 | Abnormal | 3 | 1 | DLL4 | http://www. |
| | Vein | | | | informatics.jax.org/ |
| | Development | | | | searches/Phat. |
| | | | | | cgi?id=MP:0003411 |
| 6.82 | Atretic | 3 | 1 | DLL4 | http://www. |
| | Vasculature | | | | informatics.jax.org/ |
| | | | | | searches/Phat. |
| | | | | | cgi?id=MP:0000265 |
| 6.82 | Abnormal | 3 | 1 | DLL4 | http://www. |
| | Common | | | | informatics.jax.org/ |
| | Cardinal Vein | | | | searches/Phat. |
| | Morphology | | | | cgi?id=MP:0004786 |
| 6.82 | Increased | 3 | 1 | ABCA3 | http://www. |
| | Wet-to-dry | | | | informatics.jax.org/ |
| | Lung Weight | | | | searches/Phat. |
| | Ratio | | | | cgi?id=MP:0011163 |
| 6.82 | Abnormal L5 | 3 | 1 | GDF15 | http://www. |
| | Dorsal Root | | | | informatics.jax.org/ |
| | Ganglion | | | | searches/Phat. |
| | Morphology | | | | cgi?id=MP:0001022 |

### Example 4

### WIPI1, HSPB6, MAP4, SNAP47, and PRDX5 are Potential Determinants of RVF

To elucidate the mechanisms by which the RVF-associated module may regulate RV failure, we focused on a) those genes with high connectivity to other genes (“hubs”) and b) those with high positive or negative correlations to hemodynamic indices of RVF (“drivers” or “repressors”). Of the 10 hubs, only WIPI1 was: a) differentially expressed in RV of BiV-HF hearts versus the RV of either LV-HF or NF hearts; and b) differentially expressed in RV versus LV of BiV-HF hearts (Table 6). Moreover, the expression of WIPI1 correlated with multiple RVF-associated hemodynamic indices (Table 5). WIPI1 encodes WD repeat domain phosphoinositide interacting protein 1, which plays a role in autophagy and mitophagy (Mleczak et al. 2013; Tsuyuki et al. 2014). To identify genetic drivers and repressors of RVF, we examined the correlation of each of the 279 transcripts within the RVF-associated module to RAP, RA:PCWP, MAP:RA, PASP, SBP, and CI (see Data File 1). HSPB6, SNAP47 and MAP4 emerged as significant genetic drivers of RVF, and PRDX5 as a significant genetic repressor of RVF (Table 5). Increased HSPB6, SNAP47 and MAP4 expression and decreased PRDX5 expression were associated with increased RAP, PASP, and RA:PCWP ratio and with decreased MAP:RA ratio, SBP, and CI—all hemodynamic markers of RVF. HSPB6, heat shock protein beta-6 (also known as Hsp20), is a ubiquitous small heat shock protein that is most highly expressed in skeletal, cardiac, and smooth muscle. Increased tissue expression and plasma levels of Hspb6 have been reported in patients with advanced HFrEF patients (Qian et al. 2009) and cardiomyopathic animals (Kozawa et al. 2002), respectively. Studies in isolated cardiac myocytes and transgenic mice suggest that Hspb6 plays a role in cardiac contractile function (Chu et al. 2004; Pipkin et al. 2003; Wang et al. 2009) and cardioprotection (Qian et al. 2009; Fan et al. 2005). SNAP47, a part of the intracellular membrane fusion machinery, mediates intracellular transport and vesicular secretion, but its role in non-neuronal cells is unknown (Jurado et al. 2013; Kuster et al. 2015; Shimojo et al. 2015). MAP4 encodes microtubule associated protein 4, which has been shown to be involved in microtubule stabilization (Cheng et al. 2010; Fassett et al. 2013), myogenesis (Mangan et al. 1996; Mogessie et al. 2015), myocyte metabolism (Teng et al. 2010), and inhibition of microtubule-based mRNA active transport (Scholz et al. 2006; Scholz et al. 2008). PRDX5, peroxiredoxin 5, is a ubiquitously expressed thioredoxin peroxidase and peroxynitrite reductase that can protect mitochondrial DNA from oxidative damage (Banmeyer et al. 2005; Dubuisson et al. 2004).

TABLE 5
Pearson's correlation coefficients of RVF-associated drivers, repressor, and hub with hemodynamic indices.

| | Correlation Coefficient (Pearson's p value) | | | | | | RVF |
|---|---|---|---|---|---|---|---|
| Gene | RA | RA:PCWP | MAP:RA | PASP | SBP | CI | Index |
| HSPB6 | 0.810 | 0.803 | −0.583 | 0.861 | −0.642 | −0.688 | 0.75 |
| | (<0.001) | (<0.001) | (0.023) | (<0.001) | (0.010) | (0.005) | |
| SNAP47 | 0.735 | 0.727 | −0.689 | 0.686 | −0.761 | −0.575 | 0.70 |
| | (0.002) | (0.002) | (0.005) | (0.005) | (0.001) | (0.025) | |
| MAP4 | 0.703 | 0.592 | −0.500 | 0.746 | −0.697 | −0.624 | 0.65 |
| | (0.003) | (0.020) | (0.058) | (0.001) | (0.004) | (0.013) | |
| PRDX5 | −0.679 | −0.660 | 0.600 | −0.630 | 0.602 | 0.745 | −0.64 |
| | (0.009) | (0.007) | (0.019) | (0.012) | (0.018) | (0.001) | |
| WIPI1 | 0.528 | 0.477 | −0.455 | 0.664 | −0.624 | −0.442 | 0.55 |
| | (0.043) | (0.072) | (0.088) | (0.007) | (0.013) | (0.099) | |

RA, right atrial pressure; RA:PCWP, ratio of right atrial pressure to pulmonary capillary wedge pressure; MAP:RA, ratio of mean arterial pressure to right atrial pressure; PASP, pulmonary artery systolic pressure; SBP, systolic blood pressure; CI, cardiac index; RVF, right ventricular failure; RVF index is calculated as the average of three coefficients-the correlation coefficients for RA and PASP and the negative value of the correlation coefficient for MAP:RA. Pearson's p values are presented in parentheses.

TABLE 6
Genetic hubs of RVF−associated module.

| Hub Gene | Betweenness | Significance | FPKM (Mean ± SEM) | | | |
|---|---|---|---|---|---|---|
| Name | Centrality | P-value | NF_RV | LV-HF_RV | BiV-HF_RV | NF_LV |
| SBNO2 | 0.1274 | 0 | 10.2 ± 1.4 | 9.5 ± 2.2 | 16.4 ± 5.2 | 11.3 ± 2.8 |
| TNNI3 | 0.0854 | 0 | 7280.2 ± 790.3 | 7911.4 ± 860.5 | 11788.6 ± 2719.9 | 8702.3 ± 580.7 |
| ADAP1 | 0.0558 | 0.00001 | 3.8 ± 0.8 | 5.9 ± 0.8 | 9.8 ± 2.2 | 5.1 ± 1.0 |
| RRBP1 | 0.077 | 0.00002 | 27.2 ± 2.1 | 32.5 ± 4.0 | 39.3 ± 8.5 | 30.0 ± 6.3 |
| WIPI1 | 0.0507 | 0.00002 | 32.4 ± 3.2 | 33.6 ± 1.5 | 46.2 ± 6.2 | 40.2 ± 4.0 |
| ANKRD13D | 0.0961 | 0.00004 | 7.1 ± 0.6 | 7.2 ± 1.5 | 9.3 ± 2.4 | 6.9 ± 0.6 |
| ADRA2C | 0.0584 | 0.00004 | 3.7 ± 0.9 | 3.6 ± 0.9 | 6.3 ± 1.6 | 3.4 ± 0.8 |
| JBTS26 | 0.0396 | 0.00013 | 3.8 ± 0.3 | 3.5 ± 0.3 | 6.2 ± 0.8 | 5.5 ± 1.0 |
| DRG2 | 0.0566 | 0.0002 | 12.9 ± 1.0 | 13.0 ± 0.7 | 15.1 ± 2.3 | 13.1 ± 1.1 |
| XLOC_007409 | 0.0866 | 0.00026 | 1.3 ± 0.6 | 1.2 ± 0.5 | 3.9 ± 2.2 | 0.0 ± 0.0 |

| | | | P values | | |
|---|---|---|---|---|---|
| Hub Gene | FPKM (Mean ± SEM) | | ^{*}RV (BiV-HF vs. | ^{*}LV (BiV-HF, | ^{†}BiV-HF RV |
| Name | LV-HF_LV | BiV-HF_LV | NF, LV-HF) | LVHF vs. NF) | vs. LV |
| SBNO2 | 9.1 ± 1.1 | 16.7 ± 6.5 | 0.122 | 0.769 | 0.914 |
| TNNI3 | 10755.1 ± 1651.7 | 11279.0 ± 668.9 | 0.058 | 0.094 | 0.851 |
| ADAP1 | 6.7 ± 0.8 | 8.6 ± 1.3 | 0.014 | 0.079 | 0.415 |
| RRBP1 | 35.2 ± 2.6 | 38.4 ± 12.5 | 0.176 | 0.496 | 0.854 |
| WIPI1 | 36.6 ± 3.6 | 34.8 ± 3.6 | 0.017 | 0.329 | 0.048 |
| ANKRD13D | 7.4 ± 1.6 | 9.0 ± 2.3 | 0.279 | 0.521 | 0.728 |
| ADRA2C | 3.0 ± 0.4 | 6.3 ± 1.3 | 0.077 | 0.384 | 0.947 |
| JBTS26 | 4.7 ± 0.4 | 5.3 ± 1.0 | 0.001 | 0.634 | 0.234 |
| DRG2 | 12.4 ± 1.0 | 13.5 ± 0.8 | 0.261 | 0.91 | 0.363 |
| XLOC_007409 | 0.0 ± 0.0 | 3.3 ± 2.7 | 0.112 | 0.419 | 0.359 |

FPKM, fragments per kilobase of transcript per million mapped read; NF, non-failing; LV-HF, left ventricular heart failure; BiV-HF, biventricular heart failure; RV, right ventricle; LV left ventricle.
^{*}Unpaired two-tailed Student's t-test p value, ^{†}denotes paired two-tailed Student's t-test p value.

### Example 5

### Wipi1, Hspb6, and Map4 are Upregulated Only in the Failing RV and not in the Merely Dysfunctional RV

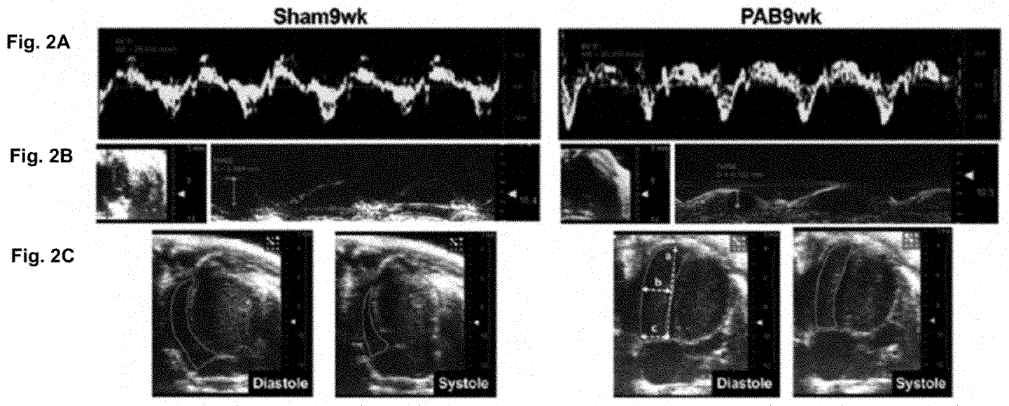

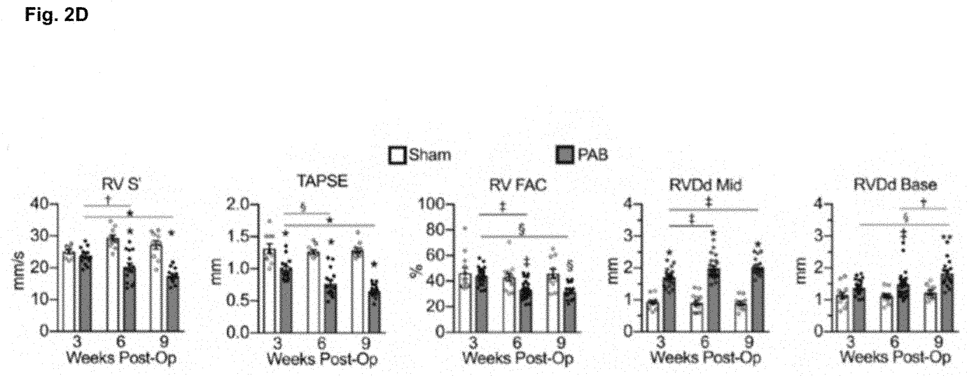

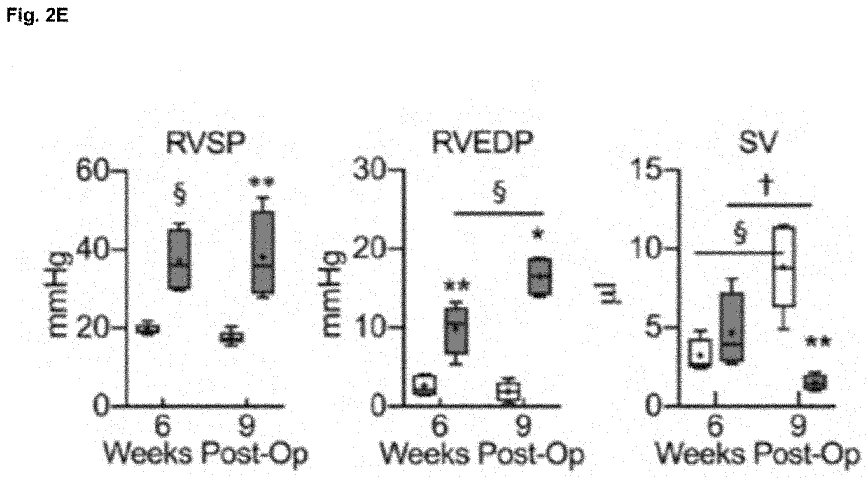

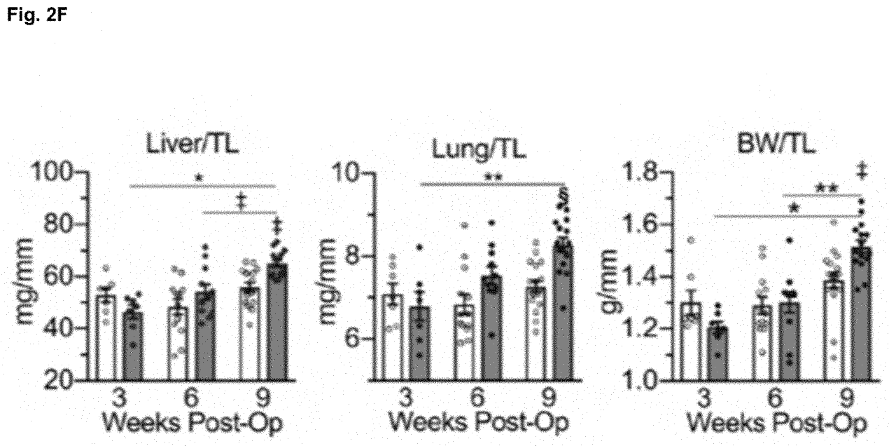

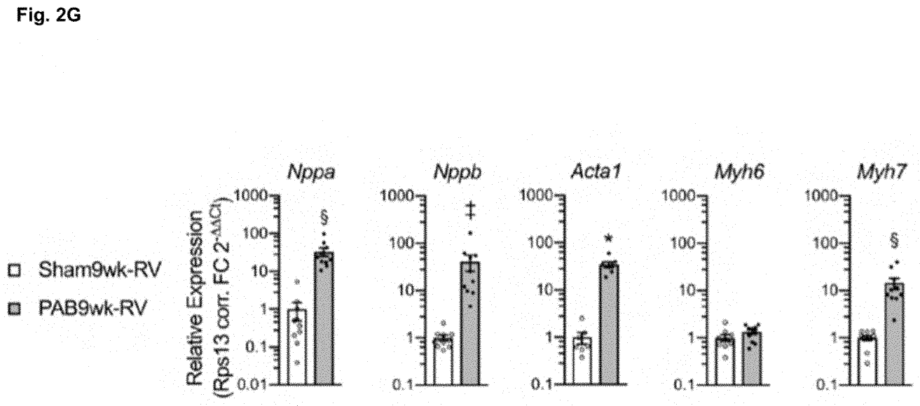

To validate their associations with RVF, we measured the ventricular expression of Wipi1, Hspb6, Snap47, Map4, and Prdx5 in a mouse model of pressure overload induced RVF. Adult male C57BL/6J mice (age 10-12 wks) were subjected to moderate pulmonary artery banding (PAB, 25g) or thoracotomy alone (Sham) and assessed at 3-wk intervals following surgery. By 3 wks post-PAB, RV systolic dysfunction and mild RV dilatation were echocardiographically evident (FIGS. 2A-2D). However, RV failure, as defined by marked peripheral edema, hepatic congestion, and pulmonary edema on terminal morphometrics, did not manifest until 9 wks post-PAB (FIGS. 2E-2F). By 9 wk post-surgery, PAB mice also developed mild pulmonary edema (increased lung weight/tibia length) and peripheral edema (increased BW/tibia length). Failing PAB9wk mice also demonstrated marked induction of the fetal gene program associated with heart failure, namely upregulation of atrial natriuretic factor (Nppa), brain natriuretic peptide (Nppb), and skeletal alpha actin (Acta1), and switching of cardiac myosin heavy chain isoforms to a predominance of βMHC, encoded by Myhc7 (FIG. 2G).

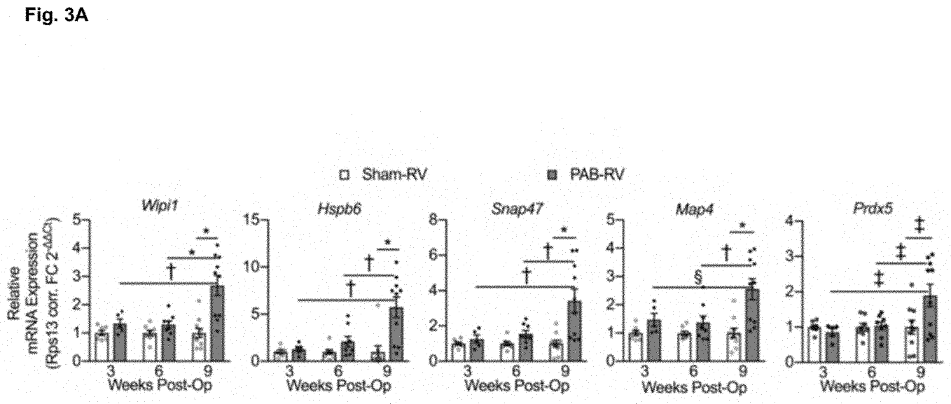

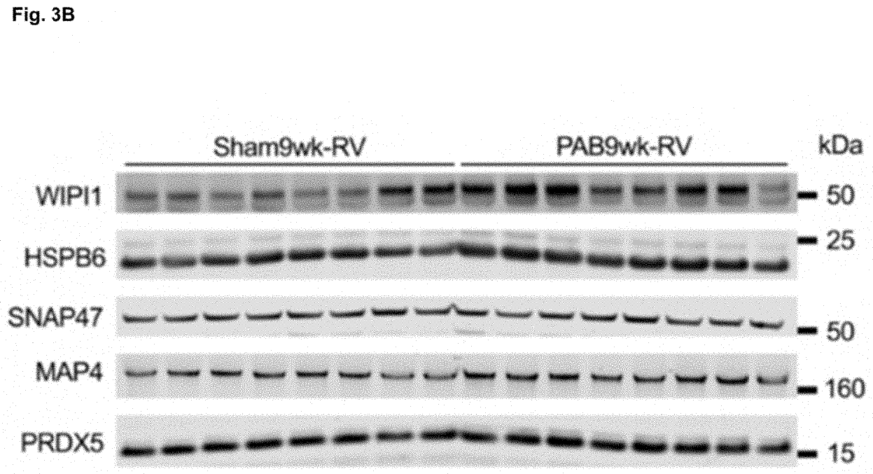

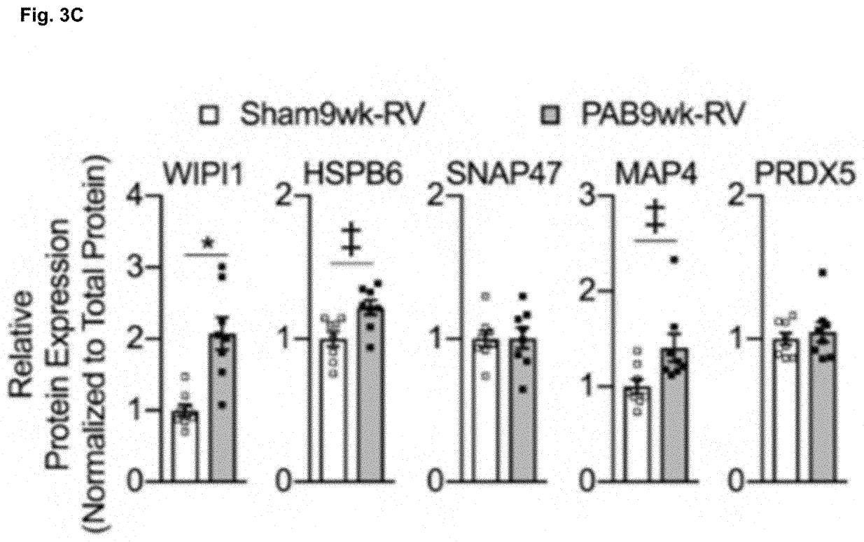

As in the human BiV-HF hearts, Wipi1, Hspb6, Snap47, and Map4 mRNA expression were increased in the failing RV of PAB mice (PAB9wk-RV) compared to that of time-matched Sham (Sham9wk-RV) (FIG. 3A). Transcriptional analyses at 3- and 6-wks post-surgery confirmed that Wipi1, Hspb6, and Map4 inductions were indeed specific to RVF (PAB9wk) and not associated with simply RV pressure overload or RV dysfunction. Western analysis confirmed increased protein expression at PAB9wk of Wipi1, Hspb6, and Map4 but not Snap47, thereby drawing into question the significance of Snap47 as a genetic driver of RVF (FIGS. 3B-3C). In contrast to the findings in human BiV-HF RV, there was no difference in Prdx5 expression between PAB9wk-RV and Sham9wk-RV, at either the transcript or protein level.

### Example 6

### Transcriptional Upregulation of Wipi1, Hspb6, and Map4 are Specific to the Failing RV and not Evidenced in the Failing LV

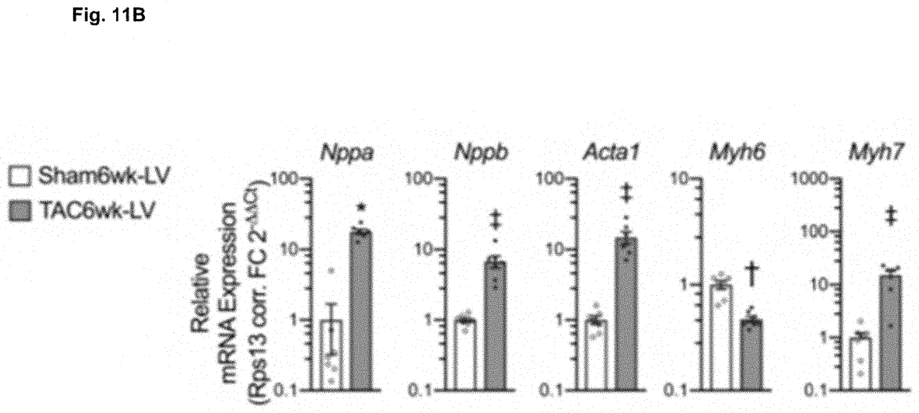

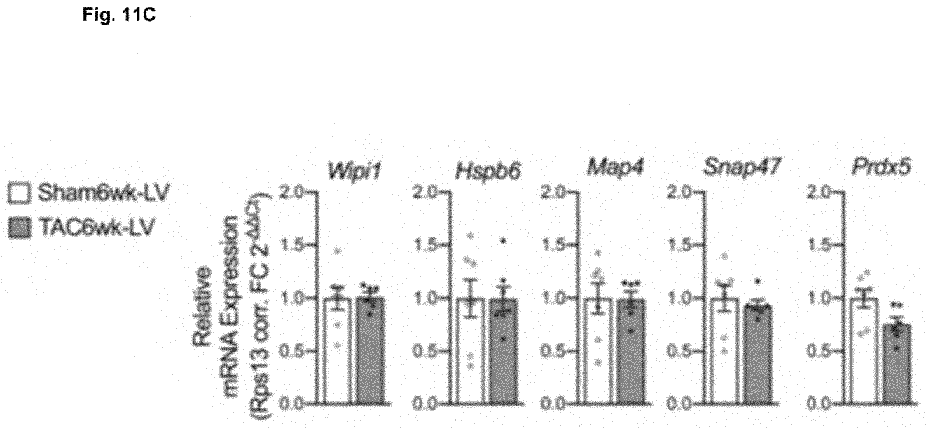

To confirm that these transcriptional changes were specific to the failing RV and not shared by the failing LV, we also assessed the expression of these gene hub and drivers/repressors in a mouse model of pressure overload induced LVF (FIGS. 11A-11C). Adult male C57BL/6J mice (age 10-12 wks) were subjected to severe transverse aortic constriction (TAC, 27g) or thoracotomy alone (Sham) and assessed at 3-wk intervals following surgery. By 3 wks post-surgery, TAC mice developed increased LV mass and depressed LV systolic function. By 6 wks post-surgery, TAC mice progressed to severe LV dysfunction, LV dilatation, and overt LVF, as manifested by severe pulmonary edema on terminal morphometric analysis; induction of the fetal gene program was also confirmed. Expressions of Wipi1, Hspb6, Snap47, Map4, and Prdx5 in the failing LV of TAC6wk mice were similar to that in LV of Sham6wk mice.

### Example 7

### In Vitro Silencing of Wipi1 Partially Protects Against RVF-Associated Neurohormone-Induced HF

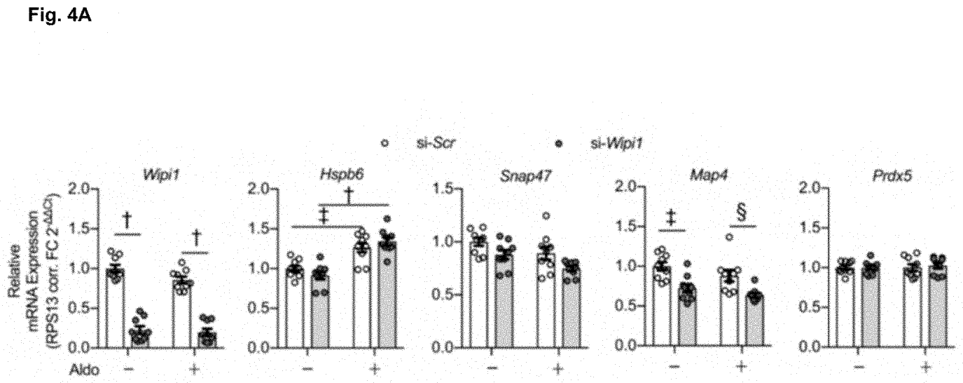

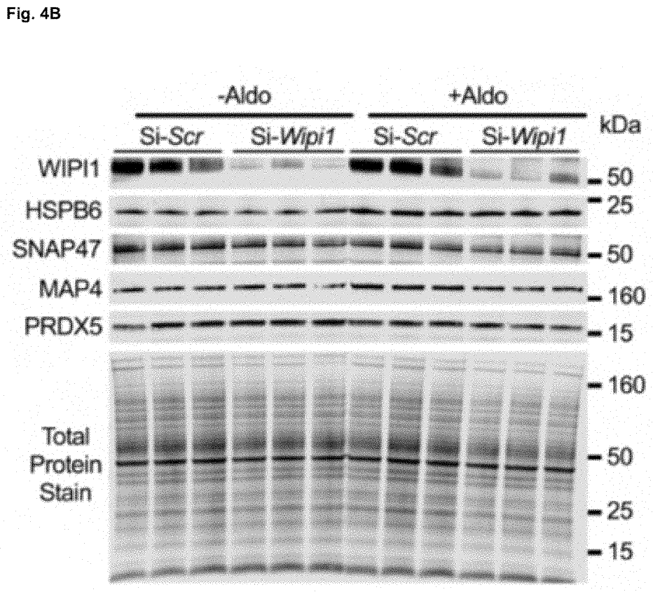

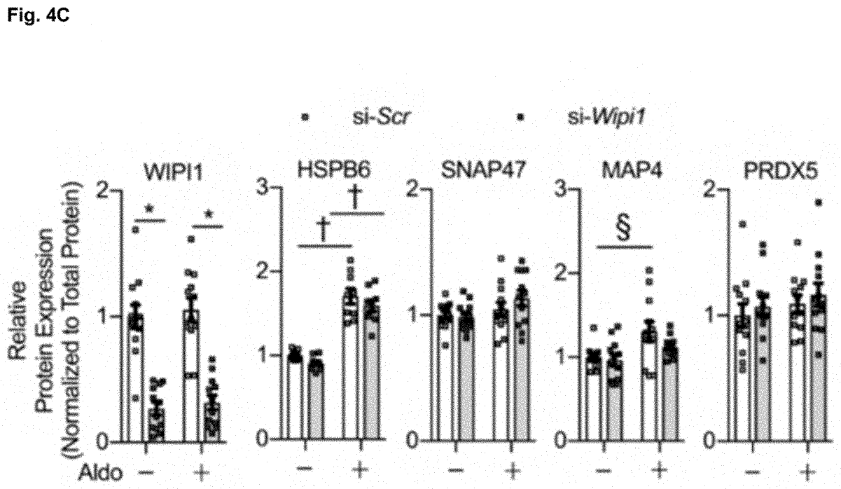

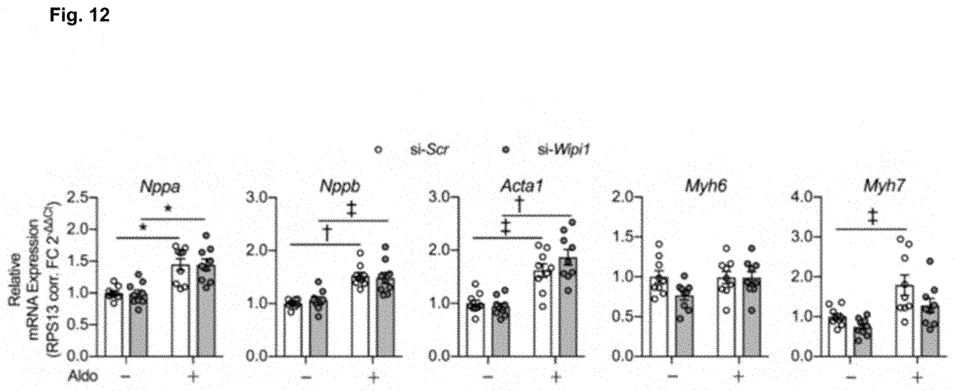

WGCNA discovered modest to strong correlations between RVF-associated hub Wipi1 and each of our candidate RVF-associated drivers and repressors, suggesting potential functional or biological interactions between them (Map4 R²=0.802, Snap47 R²=0.726, Hsbp6 R²=0.672, and Prdx5 R²=0.608.) Our in vivo RVF mouse model validated that transcriptional changes in Wipi1 correlated with that of Hspb6 and Map4, thereby raising the hypothesis that Wipi1 might potentially regulate one or both of these RVF-associated drivers. As a genetic hub of RVF, Wipi1 may be a potential target for RVF therapy. Thus, we sought to ascertain the cardioprotective potential of silencing Wipi1 in an in vitro model of heart failure and its effect on RVF-associated drivers. Specifically, we cultured isolated neonatal rat ventricular myocytes (NRVMs) with aldosterone to mimic the neurohormonal activation predominantly associated with right ventricular dysfunction and failure (Aguero et al. 2014; Gregori et al. 2014; Gregori et al. 2015; Maron et al. 2013; Safdar et al. 2015). In control NRVMs transfected with non-targeting small interfering RNA (si-scramble), neurohormonal stimulation with aldosterone significantly increased the expression of Nppa, Nppb, Acta1, and Myh7, consistent with induction of the fetal gene program in heart failure (FIGS. 11A-11C). Specific small interfering RNA (si-Wipi1) significantly knocked down Wipi1 expression in NRVMs by ˜70% at both the mRNA and protein levels (FIGS. 4A-4C). Notably, silencing Wipi1 prevented aldosterone-induced upregulation of Myh7, suggesting that silencing Wipi1 is partially protective against the neurohormone-activation associated with RVF (FIG. 12).

### Example 8

### Wipi1 Regulates Map4 Expression Under Conditions of Aldosterone Activation

To determine whether RVF hub Wipi1 might regulate Hspb6, Map4, Snap47, and/or Prdx5, we also assessed the mRNA and protein expression of these genes in si-Wipi1 versus si-scramble transfected NRVMs. The effect of silencing Wipi1 on Map4, Hspb6, Snap47, and Prdx5 varied significantly. Most interestingly, silencing Wipi1 in NRVMs decreased Map4 transcript expression by about 25-30% relative to its expression in si-scramble control NRVMs, under both basal and aldosterone stimulated conditions (FIGS. 4A-4B). Aldosterone stimulation did not affect Map4 transcript levels in either si-Wipi1 or si-scramble NRVMs. However, aldosterone did induce Map4 protein expression in si-scramble control NRVMs. This aldosterone-induced upregulation of MAP4 protein expression was not observed in si-Wipi1 NRVMs (FIG. 4C). In contrast, silencing Wipi1 had no impact on Hspb6 transcript expression. Aldosterone stimulated Hspb6 transcript expression in both si-Wipi1 and si-scramble NRVMs. Hspb6 protein expression was consistent with transcript data. Neither silencing Wipi1 nor stimulation with aldosterone affected Snap47 or Prdx5 transcript or protein expression in any fashion. Taken together, our in vivo and in vitro findings validated the calculated correlation between Wipi1 and Map4 transcripts.

### Example 9

### Wipi1 Upregulation Correlates with Increased Autophagy in the Failing RV

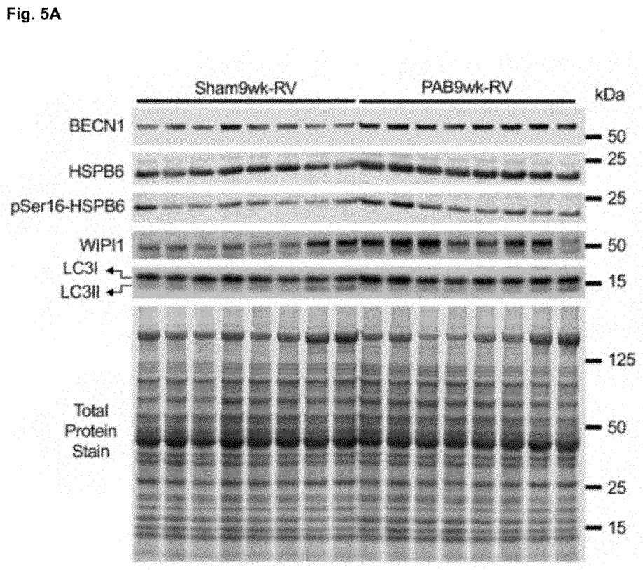

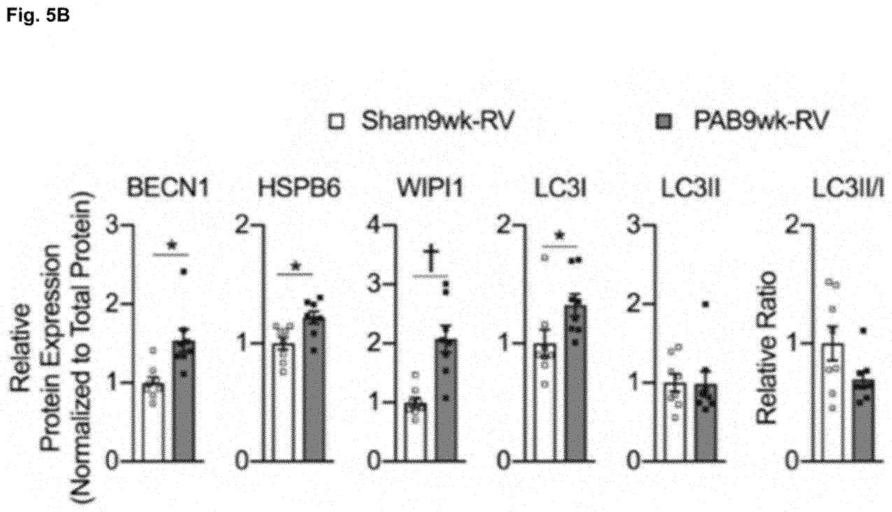

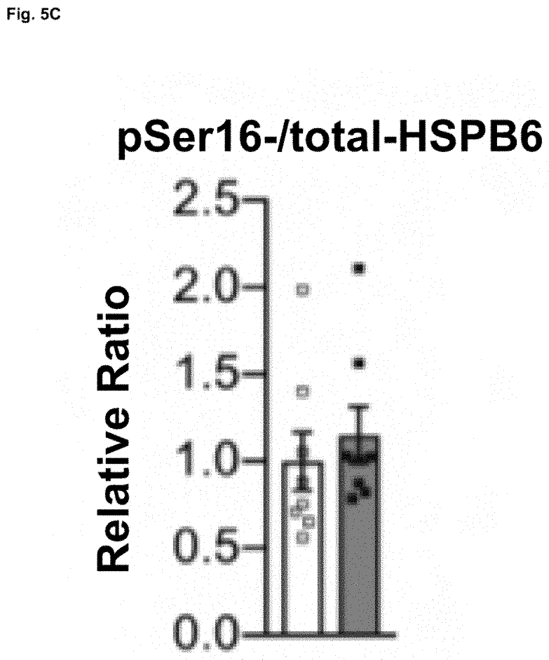

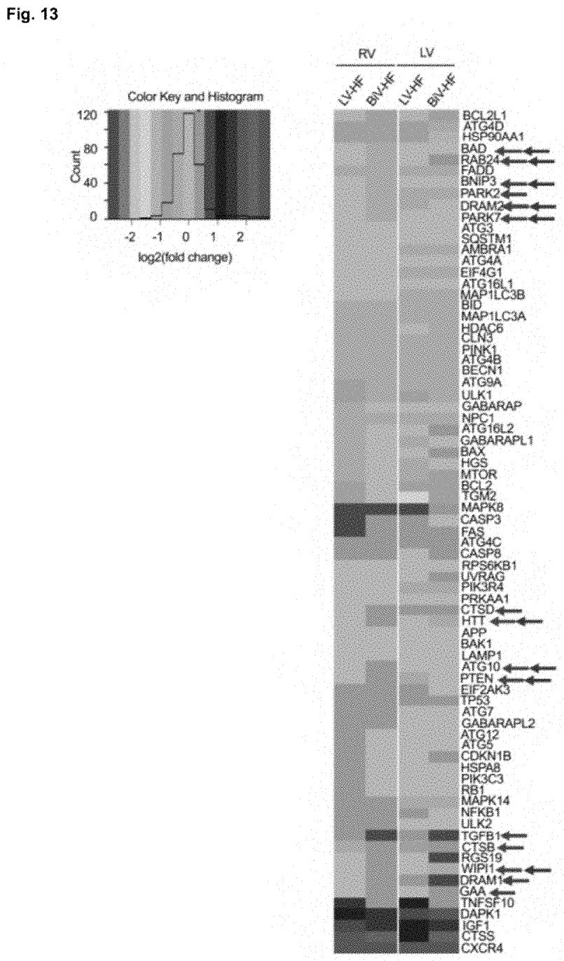

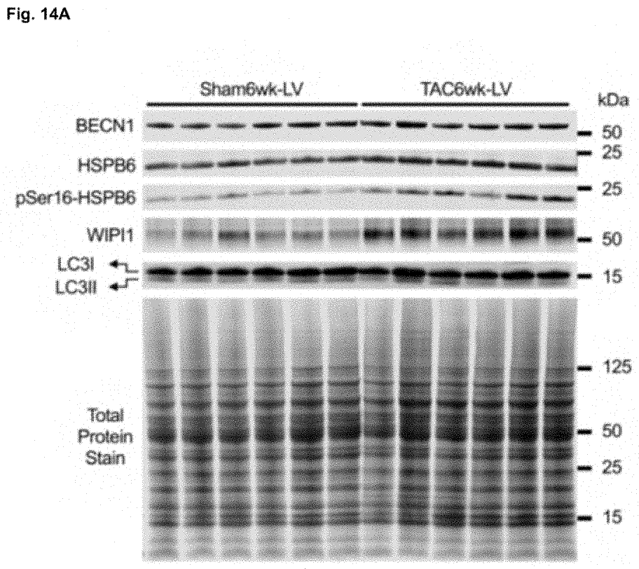

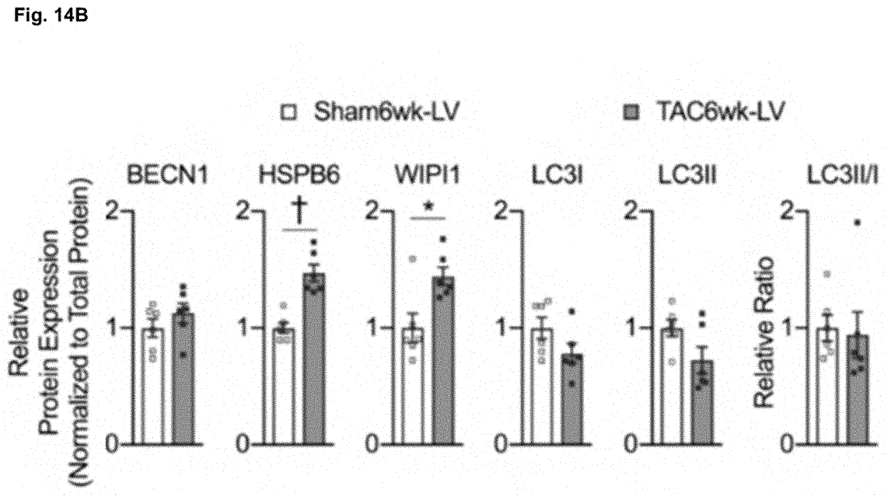

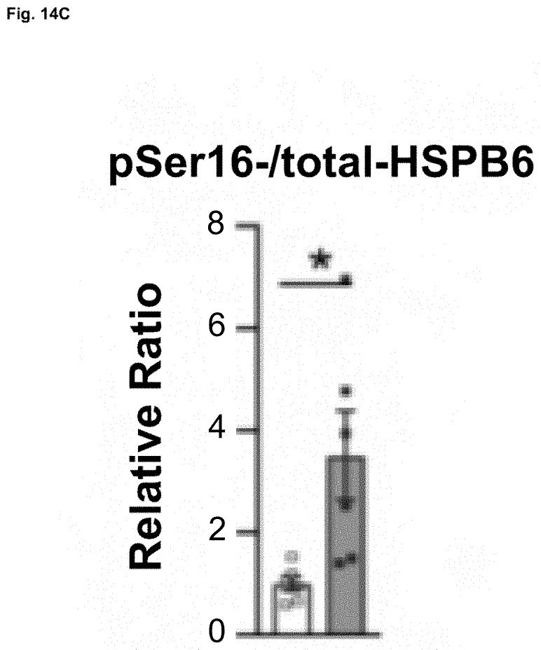

Since Wipi1 has been implicated in early autophagosome formation (Mleczak et al. 2013; Tsuyuki et al. 2014), we investigated the potential impact of Wipi1 expression on cardiac autophagy. An autophagy focused heat map of our human ventricular transcriptomic data indeed suggested differential dysregulation of autophagy pathways in the failing RV versus the failing LV (FIG. 13). To substantiate this hypothesis, we sought to characterize the differential dysregulation of autophagy in our mouse models of RV versus LV failure. We analyzed the expression of autophagy proteins beclin-1 (BECN1) and microtubule-associated protein light-chain 3 (LC3), the ratio of the phosphatidylethanolamine conjugate to the cytosolic isoform of LC3 (LC3-II/I) as an index of LC3-lipidation and autophagic flux (Tanida et al. 2008), and serine 16-phosphorylation of heat shock protein B6 (HSPB6) as a marker of canonical autophagy (Qian et al. 2009) (FIG. 5A). BECN1, HSPB6, WIPI1, and LC3I were all upregulated in PAB9wk-RV compared to Sham9wk-RV (FIG. 5B). However, LC3II expression and LC3-II/I ratio remained similar in PAB9wk-RV and Sham9wk-RV. Coupled with the finding that Ser16-phosphorylated-/total-HSPB6 ratio remained low and unchanged in PAB9wk-RV relative to Sham9wk-RV (FIG. 5C), these findings suggest that in the failing RV, non-canonical autophagy is upregulated while canonical autophagy is not. In contrast, in the failing TAC6wk-LV, both HSPB6 and its Ser16 phosphorylation were increased relative to levels in Sham6wk-LV (FIGS. 14A-14C), suggesting an upregulation of canonical autophagy in the failing LV. Hence, non-canonical autophagy may play a more significant role in the pathophysiology of RVF, while phospho-Ser16 HSPB6-mediated, BECN1-dependent, canonical autophagy is more predominant in LVF.

### Example 10

### Silencing Wipi1 Prevents Aldosterone-Induced Non-Canonical Autophagy in NRVMs

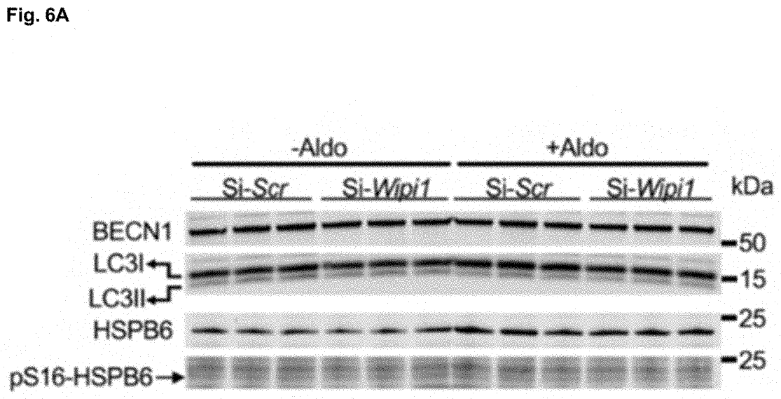

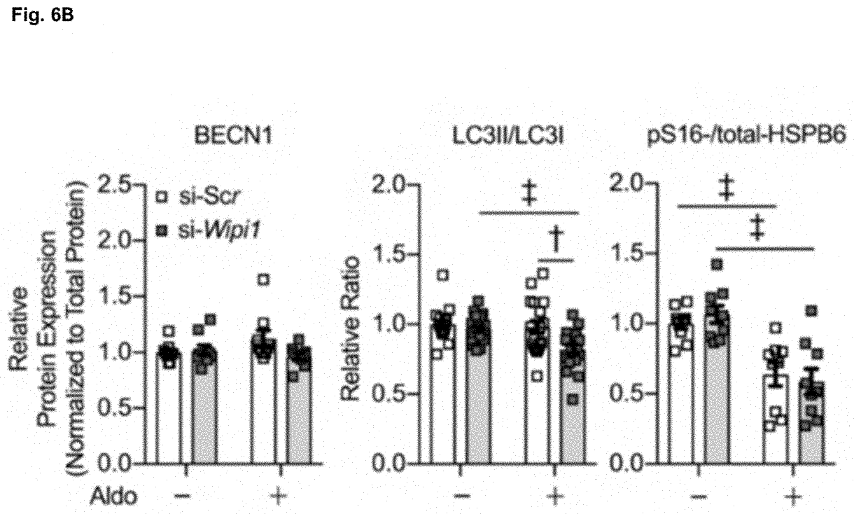

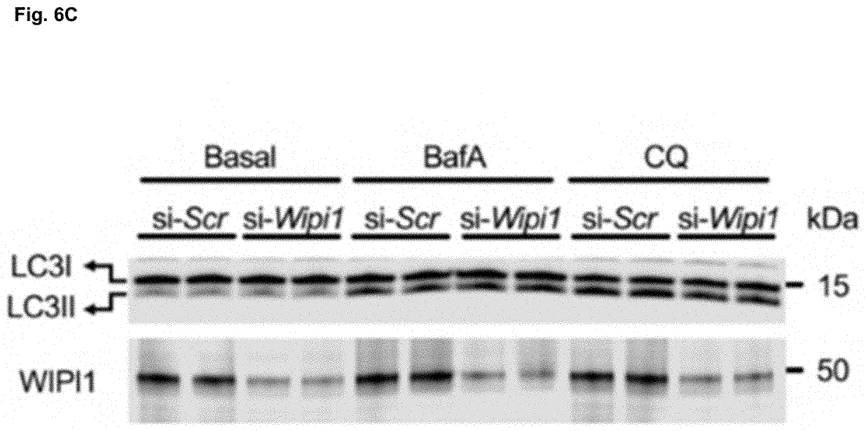

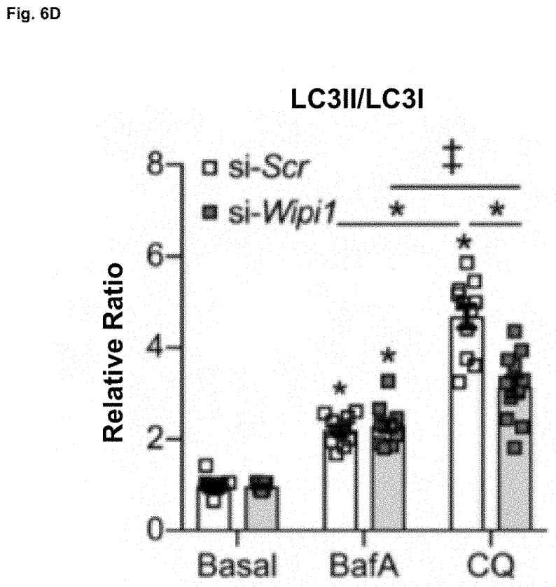

Wipi1 has been implicated in both canonical and non-canonical autophagy pathways (Codogno et al. 2012) as well as mitophagy (Lazarou et al. 2015). Given the central role for Wipi1 across multiple autophagy pathways and our findings of increased autophagic flux in the RVF mouse model, we hypothesized that the cardioprotective potential of silencing Wipi1 might be related to restoration of a physiological balance of canonical versus non-canonical autophagy or a blunting of excessive pathological autophagy. Hence, we assessed the effect of silencing Wipi1 upon the expression of Beclin1, LC3I, and LC3II as well as Ser16 phosphorylation of Hspb6 in our in vitro model of RVF-associated neurohormone activation. Isolated NRVMs were transfected with either si-scramble or si-Wipi1 and subsequently cultured for 48 h, under serum starved conditions, with or without aldosterone. In si-scramble control NRVMs, aldosterone did not affect BECN1 expression or LC3-lipidation but did decrease Ser16-HSPB6 phosphorylation in si-scramble NRVMs (FIGS. 6A-6B). These results suggest that aldosterone inhibits pSer16-HSPB6/BECN1-dependent (canonical) autophagy while increasing non-canonical autophagy in cardiac myocytes for a net constant overall autophagic flux. Under basal conditions, silencing Wipi1 had no effect on BECN1 expression, LC3 lipidation, or Ser16-HSPB6 phosphorylation. However, under aldosterone stimulation, silencing Wipi1 decreased LC3-lipidation (LC3II/I ratio) without affecting Ser16-HSPB6 phosphorylation relative to the respective si-scramble control. Hence, silencing Wipi1 could selectively limit non-canonical autophagy under conditions of chronic aldosterone activation.

To further elucidate the role of WIPI1 in canonical versus non-canonical autophagy, we used two distinct autophagy inhibitors to differentiate the effects of Wipi1 silencing on these autophagy pathways. V-ATPase inhibitor bafilomycin A (BafA) blocks LC3II lysosomal degradation during canonical autophagy. Chloroquine (CQ) inhibits the fusion between the autophagosome and lysosome, thereby rendering it capable of revealing total autophagic flux, inclusive of both canonical and non-canonical autophagy (Florey et al. 2015; Martinez-Martin et al. 2017). Thus, the difference between the effects of CQ versus BafA on LC3-II/I ratios is attributed to non-canonical autophagy. Here, we transfected NRVMs with si-Wipi1 versus si-scramble, treated them with either BafA or CQ, and then measured LC3-II/I ratios (FIG. 6C). BafA increased LC3-II/I equally in si-Wipi1 and si-scramble transfected NRVMs, suggesting that canonical autophagy remains intact even when Wipi1 is silenced. However, silencing Wipi1 blunted CQ-induced increase of LC3-II/I otherwise seen in si-scramble transfected NRVMs (FIG. 6D), suggesting that Wipi1 plays a significant role in non-canonical autophagy.

### Example 11

### Silencing Wipi1 Blunts Aldosterone Induction of Mitochondrial Superoxide and Mitochondrial Protein Oxidation

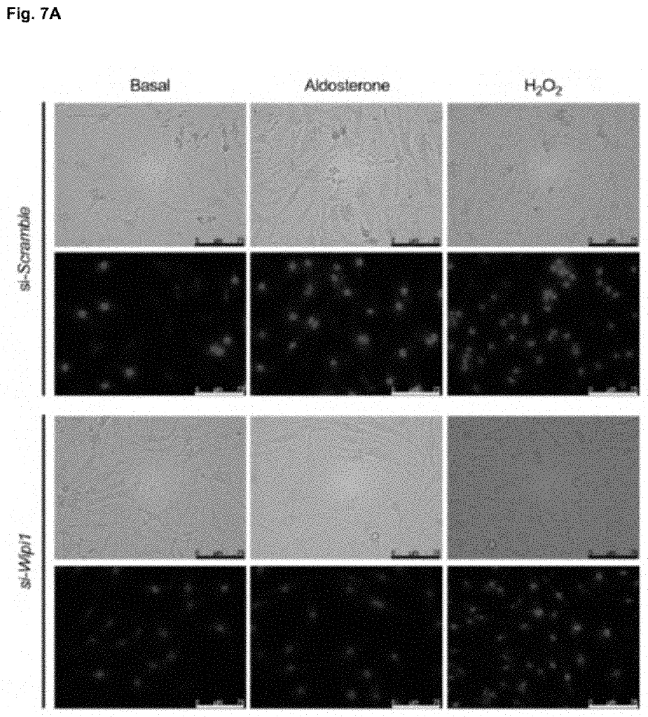

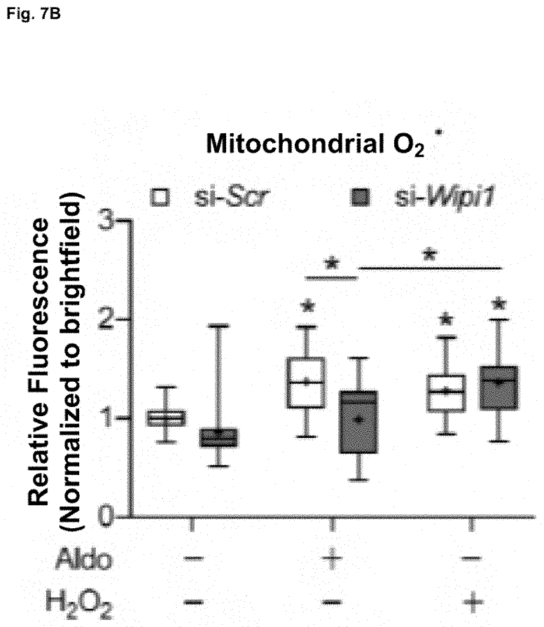

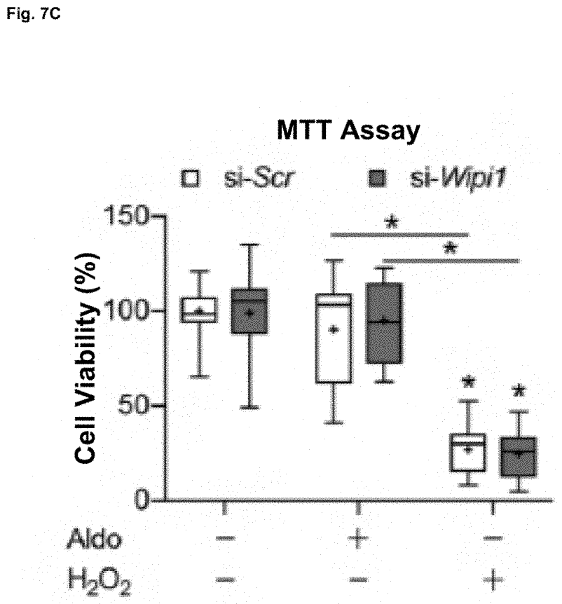

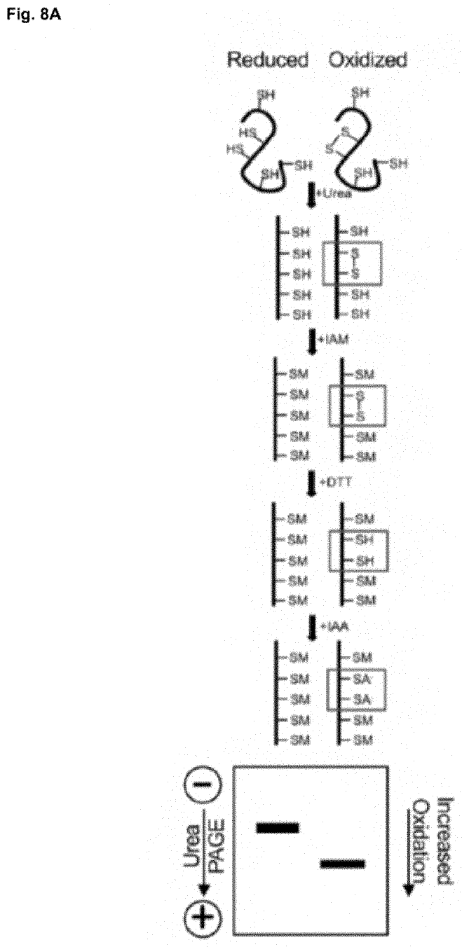

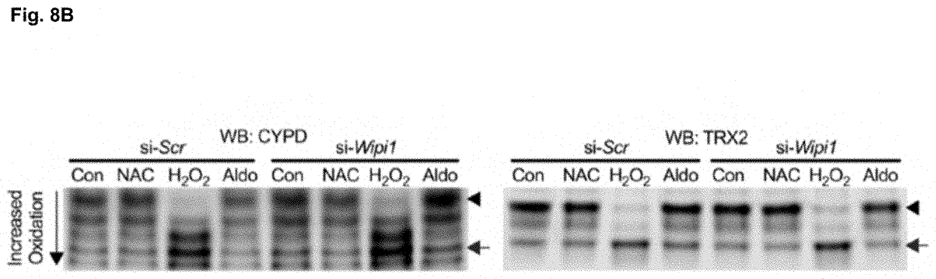

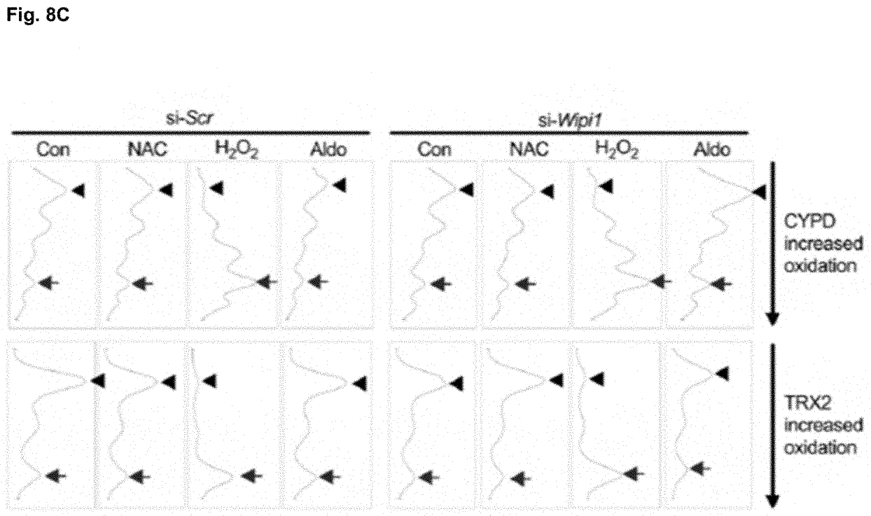

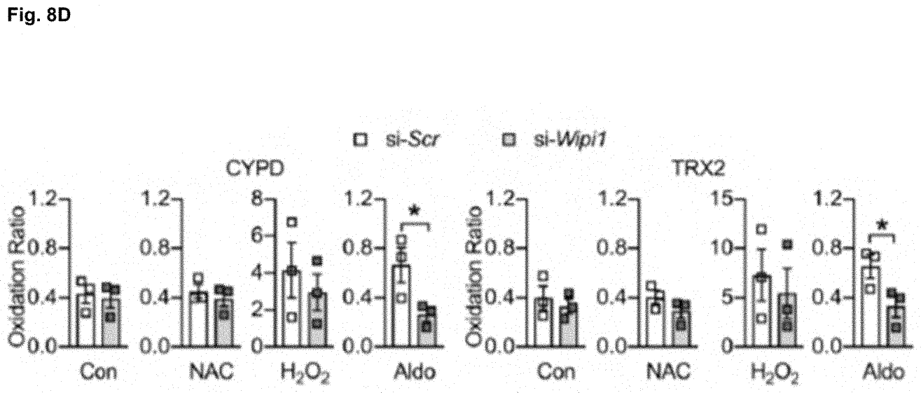

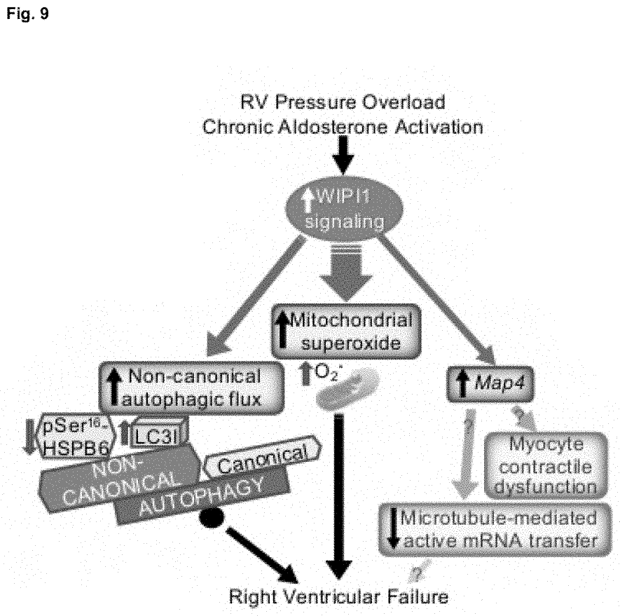

Recent studies have revealed an emerging relationship between mitochondrial oxidative stress and autophagy (Dai et al. 2011; Lee et al. 2012). Thus, we investigated the effect of silencing Wipi1 on mitochondrial superoxide levels in NRVMs subjected to aldosterone. Aldosterone increased mitochondrial superoxide levels in si-scramble control NRVMs as observed with the MitoSOX Red superoxide indicator (FIGS. 7A-7B). Silencing Wipi1 blunted aldosterone-induced mitochondrial superoxide but not that induced by hydrogen peroxide (H₂O₂). Neither aldosterone stimulation nor silencing Wipi1 negatively impacted cell viability (FIG. 7C). Moreover, subsequent assessment of the redox state of mitochondrial proteins cyclophilin D (CYPD) and thioredoxin 2 (TRX2) confirmed that silencing Wipi1 prevents downstream oxidation of these mitochondrial proteins (FIGS. 8A-8D). Silencing Wipi1 blunted aldosterone-induced oxidation of CYPD and TRX2 but had no effect on H₂O₂-induced protein oxidation (FIGS. 8B-8D). Altogether, these results indicate that silencing Wipi1 not only protects cardiac myocytes from excessive non-canonical autophagy but also mitigates mitochondrial oxidative stress by blunting mitochondrial superoxide levels and limiting mitochondrial protein oxidation.

### Example 12

### DISCUSSION

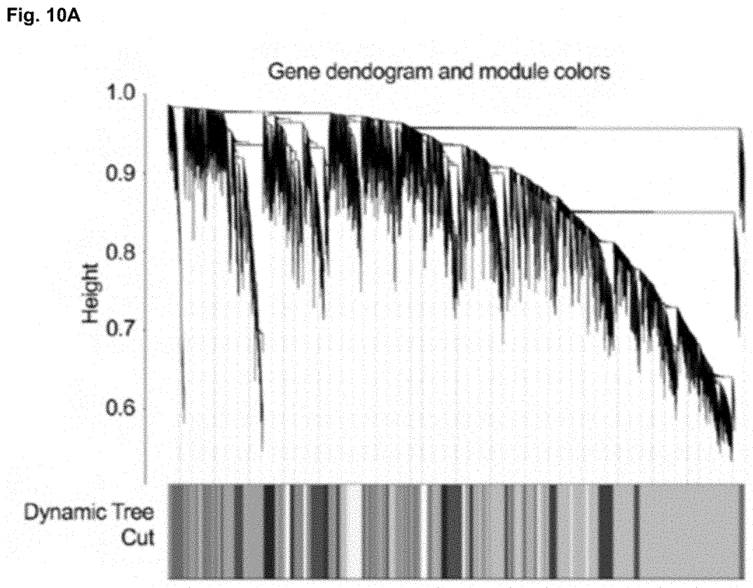

Right ventricular failure portends accelerated clinical decline and early death in patients with cardiac or pulmonary disease, and yet no therapies exist that directly target the RV. The goal of this study was to leverage human transcriptomic data to identify myocardial determinants of RV failure and experimentally validate candidate genes in in vivo and in vitro models of RVF. Using an unbiased and robust, large-scale, module-based statistical approach, we identified WIPI1 as a genetic hub of RVF, experimentally validated it in mouse models, and tested hypotheses regarding its pathophysiological role in isolated cardiac myocytes under conditions mimicking the neurohormonal activation of RVF. We provide new insights into the role of Wipi1 in non-canonical autophagy and in mitochondrial oxidative stress signaling. Our findings also offer proof of principle that silencing cardiac myocyte Wipi1 signaling holds therapeutic potential in RV failure, by preventing excessive non-canonical autophagy and blunting mitochondrial superoxide levels and mitochondrial protein oxidation (FIG. 9).

WIPI1 was first discovered for its role in nascent autophagosome formation and subsequently implicated in both canonical and non-canonical autophagy pathways (Proikas-Cezanne et al. 2015). Although cardiac myocyte autophagy has been associated with human heart failure (Hein et al. 2003; Kostin et al. 2003), the precise roles of canonical versus non-canonical autophagy in cardiovascular disease is unknown and unexplored. Transgenic mice with gain or loss of function of autophagy related genes (Matsui et al. 2007; Nakai et al. 2007; Xu et al. 2013; Zhu et al. 2007) and animal models of induced cardiac dysfunction (Qian et al. 2009; Dai et al. 2011; Matsui et al. 2007; Nakai et al. 2007; Zhu et al. 2007; Shirakabe et al. 2016; Ucar et al. 2012; Wu et al. 2017) have demonstrated both cytoprotective and pathologic roles of autophagy in the heart. Moreover, both increased and decreased autophagic flux have been associated with LV failure. Such ambiguity might be explained by differential dysregulation of canonical versus non-canonical autophagy (Codogno et al. 2012). Whereas canonical autophagosome formation involves distinct hierarchical steps that require specific ATG (autophagy-related) proteins at each stage, non-canonical autophagosome formation does not require the involvement of all ATG proteins. Non-canonical autophagosome elongation may also occur from multiple membrane sources or from pre-existing, non-phagophore endomembrane. Notably, non-canonical autophagy pathways include those that are independent of either BECN1 or LC3-lipidation. Prior cardiac autophagy studies often assessed autophagic flux via LC3-lipidation, a step universal to canonical and some non-canonical autophagy pathways as well as mitophagy, but did so without distinguishing these pathways. Both canonical and non-canonical autophagy are likely important for cellular homeostasis, and dysregulation of either or both pathways may underlie specific pathophysiological responses.

By incorporating analyses of a recently identified upstream regulator of canonical autophagy (i.e. phospho-Ser16 HSPB6) with that of LC3-lipidation, we provide original evidence suggesting that increased non-canonical autophagy distinguishes the failing RV from the failing LV. Furthermore, we demonstrated in NRVMs that silencing Wipi1 can limit aldosterone-induced non-canonical autophagy while still permitting canonical autophagy. Our finding is significant as it provides evidence that these autophagy pathways can be differentially intervened upon under pathological conditions.

Strikingly, our studies also suggest novel functions for WIPI1. We provide evidence that WIPI1 regulates mitochondrial oxidative stress signaling. Silencing Wipi1 in NRVMs decreased aldosterone-stimulated mitochondrial superoxide levels and limited oxidation of mitochondrial proteins CYPD and TRX2. The significance of this discovery is two-fold. First, the RV is more vulnerable than the LV to oxidative stress due to interventricular differences in ROS regulation (Schluter et al. 2018). Thus, attenuating mitochondrial oxidative stress, either through decreasing mitochondrial ROS production or improving antioxidant defense, is a promising therapeutic approach for RVF. Secondly, autophagy is known to regulate redox homeostasis; intracellular ROS triggers autophagy and mitophagy, which in turn modulate ROS levels. Dysregulated autophagy may potentiate detrimental ROS signaling. WIPI1 appears to lie at the nexus of autophagy and mitochondrial ROS signaling. How exactly WIPI1 regulates mitochondrial superoxide surpasses the scope of our current study but warrants further investigation.

Another novel function of WIPI1 proposed by our studies relates to its observed correlation with MAP4 (microtubule associated protein 4). WGCNA of human ventricular tissue revealed a strong correlation between WIPI1 and MAP4, which we then corroborated in our mouse model of RVF. We subsequently found that silencing Wipi1 in NRVMs reduced Map4 transcript levels, at baseline and with aldosterone stimulation. MAP4 protein expression data in the NRVMs was not as definitive; MAP4 protein levels did not mirror all the changes observed at the transcript level, perhaps reflecting differential kinetics in mRNA translation or protein degradation at baseline versus neurohormonal activation. Nevertheless, aldosterone induced MAP4 protein expression in si-Scramble NRVMs but not in si-Wipi1 NRVMs, suggesting that the correlation between Wipi1 and Map4 transcript expression is biologically significant. MAP4 is involved in microtubule stabilization (Cheng et al. 2010; Fassett et al. 2013), myogenesis (Mogessie et al. 2015), myocyte metabolism (Teng et al. 2010), and inhibition of microtubule-based mRNA active transport (Scholz et al. 2006). Overexpression of MAP4 in isolated cardiac myocytes and transgenic mice has been shown to cause cardiac myocyte contractile dysfunction (Takahashi et al. 2003). Our findings suggest that WIPI1 might regulate Map4 transcription itself and, in doing so, could theoretically impact upon MAP4-mediated cellular processes.

Our identification of HSPB6 as a driver of RV failure is consistent with current understanding of HSPB6 but also presents new insights. In our in vitro and in vivo RVF models, HSPB6 was upregulated but its phosphorylation was not. Phosphorylated HSPB6 plays important roles in cardiac contractile function (Chu et al. 2004; Pipkin et al. 2003; Wang et al. 2009), canonical autophagy (Qian et al. 2009; Liu et al. 2018), and cardioprotection (Qian et al. 2009; Fan et al. 2005). Increased cardiac expression of phosphorylated HSPB6 has been reported in the LV of advanced HFrEF patients (Qian et al. 2009), suggesting excessive canonical autophagy in the failing LV. Our finding of increased Ser16 phosphorylation of HSPB6 in the failing LV of TAC mice is consistent with this human data. Whether non-phosphorylated HSPB6 has distinct pathophysiological actions or reflects deficiencies in processes otherwise mediated by phosphorylated HSPB6 remains unclear. Our findings suggest differences between the failing RV and the failing LV with regards to HSPB6 signaling and autophagy pathways.

Above all, our study stands out for its unbiased, comprehensive approach to identifying molecular pathophysiological signaling specific to the failing RV. Prior transcriptomic analyses of animal models and human tissue have relied upon differential-expression and pathway analyses without subsequent experimental and mechanistic validation studies of proposed molecular signatures of RVF (Drake et al. 2011; Gao et al. 2006; Potus et al. 2018; Reddy et al. 2013; Urashima et al. 2008; di Salvo et al. 2015; di Salvo et al. 2015; Williams et al. 2018). Although single-gene and differential-expression analyses are powerful tools, there are a number of advantages with WGCNA (Horvath et al. 2006). As a module-based approach, WGCNA is well-suited to analyze large datasets and to take a more global view. Moreover, by examining the interaction patterns between genes to identify gene modules (networks), WGCNA filters results to a meaningful subset of the total expression data in an unsupervised, unbiased manner. WGCNA is also able to utilize and incorporate subtle shifts in gene expression, making it better able to elucidate true changes in samples compared to differential expression approaches. By correlating these modules to hemodynamic indices of RVF, we discovered a robust, biologically significant and interesting gene network. With WGCNA, we could leverage betweenness centrality to identify important actors in the alteration of a phenotype whereas such an analysis is impossible in a list of differentially expressed genes. Further investigation of intramodular connectivity between genes allowed us to identify key genetic drivers or hubs that could be experimentally validated, targeted for therapeutics, or used as novel biomarkers. Additionally, WGCNA can link novel with known genes, thereby assisting in the identification of potential functions and biological processes of novel genes. For example, WGCNA has been instrumental in identifying genetic programs critical to embryonic development (Xue et al. 2013) and cardiac myocyte differentiation (Liu et al. 2017). Finally, since WGCNA is expression rather than interaction-based, it is better able to identify large, high-impact modules driven by changes due to transcription factors and other global signaling processes as compared to an interaction-based network which excels at the exact recreation of already known pathways.

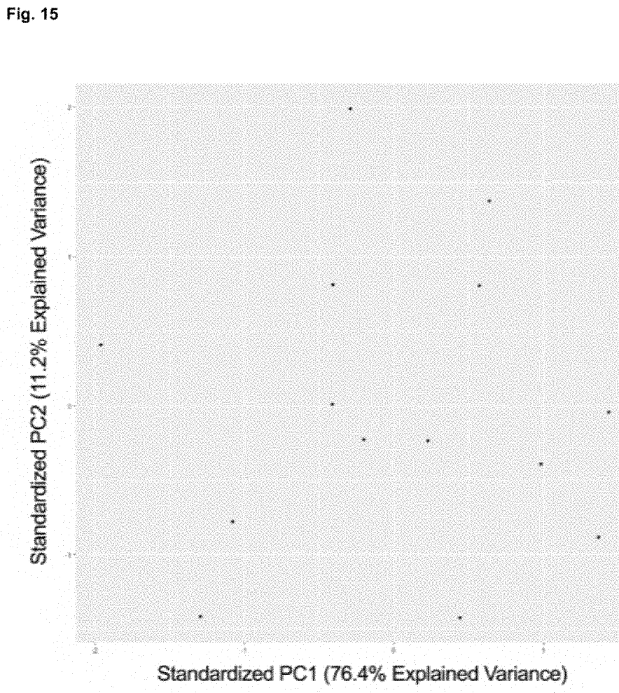

Despite its robustness for understanding global patterns that underlie phenotypic traits, WGCNA does have some limitations. Firstly, as a statistically-driven method, WGCNA may link genes that are not involved in the exact same molecular pathway. Instead, linked genes may represent related but non-interacting members of two parallel processes. Thus, experimental validation is absolutely necessary. Secondly, the WGCNA algorithm may distort the true relationships between genes and phenotypes through the use of the soft thresholding algorithm. The soft thresholding algorithm alters expressed genes by raising them to an algorithm-guided power and uses only the first principle component of a module, which may represent only a small fraction of the total variance of a module, as a proxy measure for correlation between a phenotype and the entire module. Our first principle component accounted for 77% of the total variance (FIG. 15), suggesting that these concerns do not apply to our data. Thirdly, the algorithm used by Cytoscape to generate betweenness centralities is incapable of working with weighted edges. A weighted betweenness centrality approach may be able to more accurately identify hub genes. Lastly, because our modules are derived computationally, hubs which physically interact with many genes but which do not affect their expression will not be observed in our results. Despite these limitations, WGCNA and module-phenotype analyses still offer biologically significant insights that simply cannot be afforded by single-gene and differential-expression analyses. Importantly, these limitations of WGCNA can be addressed through experimental validation and testing, as we have done.

In vitro modeling of RV failure is particularly challenging compared to that of LV failure. Morphologic and physiologic differences between the RV and LV as well as those between the pulmonary and systemic vasculatures profoundly magnify the pathophysiologic role of increased arterial elastance (decreased pulmonary vascular compliance) in RV failure (Thenappan et al. 2016). As suggested by our mouse model, chronicity of both pressure overload and neurohormonal activation are likely important determinants of WIPI1 expression. Thus limitations inherent to in vitro cell culture models might explain why Wipi1 transcript and protein expression were upregulated in the failing RV of PAB mice but not in aldosterone-stimulated NRVMs. Despite this discrepancy, our in vitro studies with si-Wipi1 still revealed a significant functional role of WIPI1 in mediating aldosterone-induced mitochondrial superoxide levels. Importantly, others have already demonstrated that hyperaldosteronism is associated with RV failure in large animal models (Aguero et al. 2014) and patients (Maron et al. 2013). Moreover, mitochondrial ROS play a significant role in the pathophysiology of RV failure but not that of LV failure (Redout et al. 2007).

Silencing Wipi1 had the greatest effects on aldosterone-induced mitochondrial superoxide signaling and non-canonical autophagy but only a small impact on the fetal gene program. This highlights the limitation of using the fetal gene program as a surrogate outcome for RV failure, rather than necessarily a limitation of the therapeutic potential of targeting Wipi1. Given the known pathophysiological roles of aldosterone and mitochondrial ROS in RV failure, the substantial blunting of aldosterone-induced mitochondrial superoxide levels and mitochondrial protein oxidation by si-Wipi1 is particularly significant.

In summary, our data demonstrate novel roles for WIPI1 in regulating mitochondrial oxidative stress signaling, non-canonical autophagy, and MAP4 transcription in RV failure. Silencing Wipi1 in an in vitro model of RVF-associated neurohormone activation decreased mitochondrial superoxide levels and mitochondrial protein oxidation, dampened excessive non-canonical autophagy, and reduced Map4 mRNA expression.

### Example 13

### Silencing Wipi1 In Vivo

To confirm the therapeutic potential of silencing or inhibiting Wipi1 in right ventricular (RV) failure, the RV-specific deletion of Wipi1 is achieved by using two adeno-associated virus (AAV) serotype 9 vectors, termed AAV9-1 and AAV9-2.

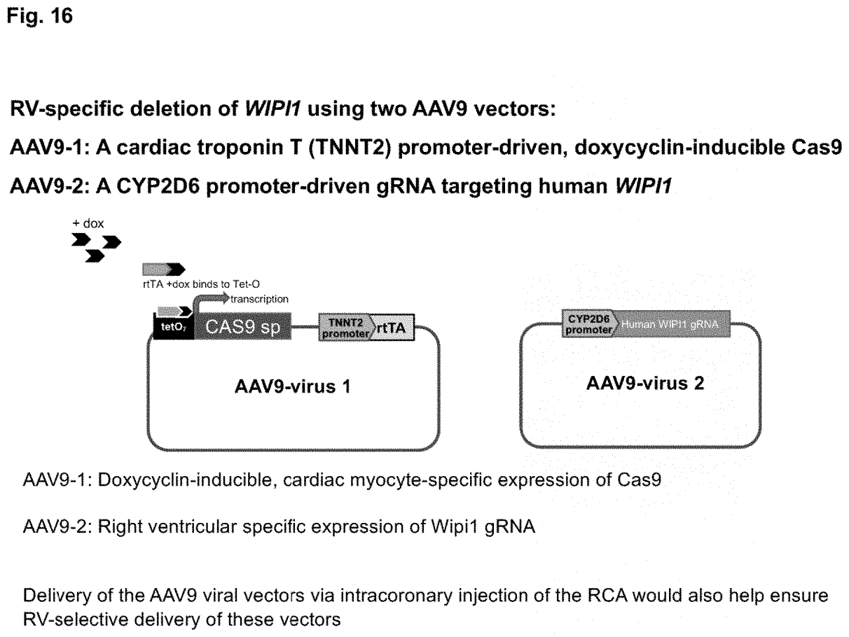

As shown in FIG. 16, AAV9-1 is constructed as a cardiac troponin T (TNNT2) promoter-driven, doxycycline-inducible Cas9 expression system, which comprises a tetracycline response element (TRE), a transgene encoding Cas9, a TNNT2 promoter region, and a transgene encoding reverse tetracycline-controlled transactivator (rtTA). This Tet-on inducible system is based on rtTA, a fusion protein comprised of the TetR repressor and the VP16 transactivation domain. A four amino acid change in the tetR DNA binding moiety alters rtTA's binding characteristics such that it can only recognize the Tet-O sequences in the TRE of the target transgene in the presence of the doxycycline (Dox) effector. The Tet-On system allows tissue-specific promoters to drive rtTA expression, resulting in tissue-specific expression of the TRE-regulated target transgene. Thus, in this AAV9-1 vector, cardiac myocyte specificity is achieved by using the TNNT2 promoter to drive rtTA expression. Upon doxycycline administration, doxycycline binds to rtTA, which then binds to Tet-O, thereby initiating the transcription of Cas9.

AAV9-2 is constructed as a CYP2D6 promoter driven system expressing gRNA targeting human WIPI1. This construct is comprised of a CYP2D6 promoter region and a transgene encoding gRNA that is specific for WIPI1 (FIG. 16). CYP2D6 promoter is selected because of previous reports on RV-specific expression of CYP2D6.

AAV9-1 and AAV9-2 are administered directly into the right coronary artery (RCA) of the subject via intracoronary injection (cardiac catheterization), to further ensure RV-selective delivery of these viral vectors. Once these vectors transfect into cardiac myocytes, human WIPI1 gRNA and doxycycline-induced Cas9 are expressed and form the Cas9:gRNA complex, which then binds to the human WIPI1 gene and deletes it from the genome. Knockout of WIPI1 gene is expected to substantially blunt aldosterone-induced mitochondrial superoxide levels and mitochondrial protein oxidation, dampen excessive non-canonical autophagy, and reduce Map4 mRNA expression, in the subject, which will confirm a RV-specific therapy for cardiopulmonary disease in, e.g., humans.

### DOCUMENTS CITED

1. Blauwet L A, Delgado-Montero A, Ryo K, Marek J J, Alharethi R, Mather P J, et al. Right Ventricular Function in Peripartum Cardiomyopathy at Presentation Is Associated With Subsequent Left Ventricular Recovery and Clinical Outcomes. Circ Heart Fail. 2016; 9(5).
2. Campbell P, Takeuchi M, Bourgoun M, Shah A, Foster E, Brown M W, et al. Right ventricular function, pulmonary pressure estimation, and clinical outcomes in cardiac resynchronization therapy. Circ Heart Fail. 2013; 6(3):435-42.
3. Cenkerova K, Dubrava J, Pokorna V, Kaluzay J, and Jurkovicova O. Right ventricular systolic dysfunction and its prognostic value in heart failure with preserved ejection fraction. Acta Cardiol. 2015; 70(4):387-93.
4. Dini F L, Conti U, Fontanive P, Andreini D, Banti S, Braccini L, et al. Right ventricular dysfunction is a major predictor of outcome in patients with moderate to severe mitral regurgitation and left ventricular dysfunction. Am Heart J. 2007; 154(1):172-9.
5. Drazner M H, Velez-Martinez M, Ayers C R, Reimold S C, Thibodeau J T, Mishkin J D, et al. Relationship of right- to left-sided ventricular filling pressures in advanced heart failure: insights from the ESCAPE trial. Circ Heart Fail. 2013; 6(2):264-70.
6. Gerges M, Gerges C, Pistritto A M, Lang M B, Trip P, Jakowitsch J, et al. Pulmonary Hypertension in Heart Failure. Epidemiology, Right Ventricular Function, and Survival. Am J Respir Crit Care Med. 2015; 192(10):1234-46.
7. Ghio S, Temporelli P L, Klersy C, Simioniuc A, Girardi B, Scelsi L, et al. Prognostic relevance of a non-invasive evaluation of right ventricular function and pulmonary artery pressure in patients with chronic heart failure. Eur J Heart Fail. 2013; 15(4):408-14.
8. Gulati A, Ismail T F, Jabbour A, Alpendurada F, Guha K, Ismail N A, et al. The prevalence and prognostic significance of right ventricular systolic dysfunction in nonischemic dilated cardiomyopathy. Circulation. 2013; 128(15):1623-33.
9. Kim H, Jung C, Yoon H J, Park H S, Cho Y K, Nam C W, et al. Prognostic value of tricuspid annular tissue Doppler velocity in heart failure with atrial fibrillation. J Am Soc Echocardiogr. 2012; 25(4):436-43.
10. Kusunose K, Phelan D, Seicean S, Seicean A, Collier P, Boden K A, et al. Relation of Echocardiographic Characteristics of the Right-Sided Heart With Incident Heart Failure and Mortality in Patients With Sleep-Disordered Breathing and Preserved Left Ventricular Ejection Fraction. Am J Cardiol. 2016; 118(8):1268-73.
11. Olson J, Samad B A, and Alam M. The prognostic significance of right ventricular tissue Doppler parameters in patients with left ventricular systolic heart failure: an observational cohort study. Heart. 2012; 98(15):1142-5.
12. Park S J, Park J H, Lee H S, Kim M S, Park Y K, Park Y, et al. Impaired RV global longitudinal strain is associated with poor long-term clinical outcomes in patients with acute inferior STEMI. JACC Cardiovasc Imaging. 2015; 8(2):161-9.
13. Forfia P R, Fisher M R, Mathai S C, Housten-Harris T, Hemnes A R, Borlaug B A, et al. Tricuspid annular displacement predicts survival in pulmonary hypertension. Am J Respir Crit Care Med. 2006; 174(9):1034-41.
14. Grapsa J, Pereira Nunes M C, Tan T C, Cabrita I Z, Coulter T, Smith B C, et al. Echocardiographic and Hemodynamic Predictors of Survival in Precapillary Pulmonary Hypertension: Seven-Year Follow-Up. Circ Cardiovasc Imaging. 2015; 8(6).
15. Haddad F, Spruijt O A, Denault A Y, Mercier O, Brunner N, Furman D, et al. Right Heart Score for Predicting Outcome in Idiopathic, Familial, or Drug- and Toxin-Associated Pulmonary Arterial Hypertension. JACC Cardiovasc Imaging. 2015; 8(6):627-38.
16. van de Veerdonk M C, Kind T, Marcus J T, Mauritz G J, Heymans M W, Bogaard H J, et al. Progressive right ventricular dysfunction in patients with pulmonary arterial hypertension responding to therapy. J Am Coll Cardiol. 2011; 58(24):2511-9.
17. Damy T, Kallvikbacka-Bennett A, Goode K, Khaleva O, Lewinter C, Hobkirk J, et al. Prevalence of, associations with, and prognostic value of tricuspid annular plane systolic excursion (TAPSE) among out-patients referred for the evaluation of heart failure. J Card Fail. 2012; 18(3):216-25.
18. Gorter T M, Hoendermis E S, van Veldhuisen D J, Voors A A, Lam C S, Geelhoed B, et al. Right ventricular dysfunction in heart failure with preserved ejection fraction: a systematic review and meta-analysis. Eur J Heart Fail. 2016; 18(12):1472-87.
19. Mohammed S F, Hussain I, AbouEzzeddine O F, Takahama H, Kwon S H, Forfia P, et al. Right ventricular function in heart failure with preserved ejection fraction: a community-based study. Circulation. 2014; 130(25):2310-20.
20. Puwanant S, Priester T C, Mookadam F, Bruce C J, Redfield M M, and Chandrasekaran K. Right ventricular function in patients with preserved and reduced ejection fraction heart failure. Eur J Echocardiogr. 2009; 10(6):733-7.
21. Ribeiro A, Lindmarker P, Johnsson H, Juhlin-Dannfelt A, and Jorfeldt L. Pulmonary embolism: one-year follow-up with echocardiography doppler and five-year survival analysis. Circulation. 1999; 99(10):1325-30.
22. Sista A K, Miller L E, Kahn S R, and Kline J A. Persistent right ventricular dysfunction, functional capacity limitation, exercise intolerance, and quality of life impairment following pulmonary embolism: Systematic review with meta-analysis. Vasc Med. 2017; 22(1):37-43.
23. Collum S D, Amione-Guerra J, Cruz-Solbes A S, DiFrancesco A, Hernandez A M, Hanmandlu A, et al. Pulmonary Hypertension Associated with Idiopathic Pulmonary Fibrosis: Current and Future Perspectives. Can Respir J. 2017; 2017:1430350.
24. Kolb T M, and Hassoun P M. Right ventricular dysfunction in chronic lung disease. Cardiol Clin. 2012; 30(2):243-56.
25. Vizza C D, Lynch J P, Ochoa L L, Richardson G, and Trulock E P. Right and left ventricular dysfunction in patients with severe pulmonary disease. Chest. 1998; 113(3):576-83.
26. Rudski L G, Lai W W, Afilalo J, Hua L, Handschumacher M D, Chandrasekaran K, et al. Guidelines for the echocardiographic assessment of the right heart in adults: a report from the American Society of Echocardiography endorsed by the European Association of Echocardiography, a registered branch of the European Society of Cardiology, and the Canadian Society of Echocardiography. J Am Soc Echocardiogr. 2010; 23(7):685-713; quiz 86-8.
27. Swift A J, Rajaram S, Condliffe R, Capener D, Hurdman J, Elliot C A, et al. Diagnostic accuracy of cardiovascular magnetic resonance imaging of right ventricular morphology and function in the assessment of suspected pulmonary hypertension results from the ASPIRE registry. J Cardiovasc Magn Reson. 2012; 14:40.
28. Kato T S, Stevens G R, Jiang J, Schulze P C, Gukasyan N, Lippel M, et al. Risk stratification of ambulatory patients with advanced heart failure undergoing evaluation for heart transplantation. J Heart Lung Transplant. 2013; 32(3):333-40.
29. Courand P Y, Pina Jomir G, Khouatra C, Scheiber C, Turquier S, Glerant J C, et al. Prognostic value of right ventricular ejection fraction in pulmonary arterial hypertension. Eur Respir J. 2015; 45(1):139-49.
30. Konstantinides S V, Vicaut E, Danays T, Becattini C, Bertoletti L, Beyer-Westendorf J, et al. Impact of Thrombolytic Therapy on the Long-Term Outcome of Intermediate-Risk Pulmonary Embolism. J Am Coll Cardiol. 2017; 69(12):1536-44.
31. McLaughlin V V, Presberg K W, Doyle R L, Abman S H, McCrory D C, Fortin T, et al. Prognosis of pulmonary arterial hypertension: ACCP evidence-based clinical practice guidelines. Chest. 2004; 126(1 Suppl):78S-92S.
32. Gall H, Felix J F, Schneck F K, Milger K, Sommer N, Voswinckel R, et al. The Giessen Pulmonary Hypertension Registry: Survival in pulmonary hypertension subgroups. J Heart Lung Transplant. 2017; 36(9):957-67.
33. Yancy C W, Jessup M, Bozkurt B, Butler J, Casey D E, Jr., Drazner M H, et al. 2013 ACCF/AHA guideline for the management of heart failure: executive summary: a report of the American College of Cardiology Foundation/American Heart Association Task Force on practice guidelines. Circulation. 2013; 128(16):1810-52.
34. Haddad F, Hunt S A, Rosenthal D N, and Murphy D J. Right ventricular function in cardiovascular disease, part I: Anatomy, physiology, aging, and functional assessment of the right ventricle. Circulation. 2008; 117(11):1436-48.
35. Haddad F, Doyle R, Murphy D J, and Hunt S A. Right ventricular function in cardiovascular disease, part II: pathophysiology, clinical importance, and management of right ventricular failure. Circulation. 2008; 117(13):1717-31.
36. Zaffran S, Kelly R G, Meilhac S M, Buckingham M E, and Brown N A. Right ventricular myocardium derives from the anterior heart field. Circulation research. 2004; 95(3):261-8.
37. Drake J I, Bogaard H J, Mizuno S, Clifton B, Xie B, Gao Y, et al. Molecular signature of a right heart failure program in chronic severe pulmonary hypertension. Am J Respir Cell Mol Biol. 2011; 45(6):1239-47.
38. Gao Z, Xu H, DiSilvestre D, Halperin V L, Tunin R, Tian Y, et al. Transcriptomic profiling of the canine tachycardia-induced heart failure model: global comparison to human and murine heart failure. J Mol Cell Cardiol. 2006; 40(1):76-86.
39. Reddy S, Zhao M, Hu D Q, Fajardo G, Katznelson E, Punn R, et al. Physiologic and molecular characterization of a murine model of right ventricular volume overload. Am J Physiol Heart Circ Physiol. 2013; 304(10):H1314-27.
40. Urashima T, Zhao M, Wagner R, Fajardo G, Farahani S, Quertermous T, et al. Molecular and physiological characterization of RV remodeling in a murine model of pulmonary stenosis. Am J Physiol Heart Circ Physiol. 2008; 295(3):H1351-H68.
41. di Salvo T G, Yang K C, Brittain E, Absi T, Maltais S, and Hemnes A. Right ventricular myocardial biomarkers in human heart failure. J Card Fail. 2015; 21(5):398-411.
42. Di Salvo T G, Guo Y, Su Y R, Clark T, Brittain E, Absi T, et al. Right ventricular long noncoding RNA expression in human heart failure. Pulm Circ. 2015; 5(1):135-61.
43. Williams J L, Cavus O, Loccoh E C, Adelman S, Daugherty J C, Smith S A, et al. Defining the molecular signatures of human right heart failure. Life Sci. 2018; 196:118-26.
44. Khatri P, Sirota M, and Butte A J. Ten years of pathway analysis: current approaches and outstanding challenges. PLoS Comput Biol. 2012; 8(2):e1002375.
45. Langfelder P, and Horvath S. WGCNA: an R package for weighted correlation network analysis. BMC Bioinformatics. 2008; 9:559.
46. Mleczak A, Millar S, Tooze S A, Olson M F, and Chan E Y. Regulation of autophagosome formation by Rho kinase. Cell Signal. 2013; 25(1):1-11.
47. Tsuyuki S, Takabayashi M, Kawazu M, Kudo K, Watanabe A, Nagata Y, et al. Detection of WIPI1 mRNA as an indicator of autophagosome formation. Autophagy. 2014; 10(3):497-513.
48. Qian J, Ren X, Wang X, Zhang P, Jones W K, Molkentin J D, et al. Blockade of Hsp20 phosphorylation exacerbates cardiac ischemia/reperfusion injury by suppressed autophagy and increased cell death. Circulation research. 2009; 105(12): 1223-31.
49. Kozawa O, Matsuno H, Niwa M, Hatakeyama D, Oiso Y, Kato K, et al. HSP20, low-molecular-weight heat shock-related protein, acts extracellularly as a regulator of platelet functions: a novel defense mechanism. Life Sci. 2002; 72(2):113-24.
50. Chu G, Egnaczyk G F, Zhao W, Jo S H, Fan G C, Maggio J E, et al. Phosphoproteome analysis of cardiomyocytes subjected to beta-adrenergic stimulation: identification and characterization of a cardiac heat shock protein p20. Circulation research. 2004; 94(2):184-93.
51. Pipkin W, Johnson J A, Creazzo T L, Burch J, Komalavilas P, and Brophy C. Localization, macromolecular associations, and function of the small heat shock-related protein HSP20 in rat heart. Circulation. 2003; 107(3):469-76.
52. Wang X, Zingarelli B, O'Connor M, Zhang P, Adeyemo A, Kranias E G, et al. Overexpression of Hsp20 prevents endotoxin-induced myocardial dysfunction and apoptosis via inhibition of NF-kappaB activation. J Mol Cell Cardiol. 2009; 47(3):382-90.
53. Fan G C, Ren X, Qian J, Yuan Q, Nicolaou P, Wang Y, et al. Novel cardioprotective role of a small heat-shock protein, Hsp20, against ischemia/reperfusion injury. Circulation. 2005; 111(14):1792-9.
54. Jurado S, Goswami D, Zhang Y, Molina A J, Sudhof T C, and Malenka R C. LTP requires a unique postsynaptic SNARE fusion machinery. Neuron. 2013; 77(3):542-58.
55. Kuster A, Nola S, Dingli F, Vacca B, Gauchy C, Beaujouan J C, et al. The Q-soluble N-Ethylmaleimide-sensitive Factor Attachment Protein Receptor (Q-SNARE) SNAP-47 Regulates Trafficking of Selected Vesicle-associated Membrane Proteins (VAMPs). J Biol Chem. 2015; 290(47):28056-69.
56. Shimojo M, Courchet J, Pieraut S, Torabi-Rander N, Sando R, 3rd, Polleux F, et al. SNAREs Controlling Vesicular Release of BDNF and Development of Callosal Axons. Cell Rep. 2015; 11(7):1054-66.
57. Cheng G, Takahashi M, Shunmugavel A, Wallenborn J G, DePaoli-Roach A A, Gergs U, et al. Basis for MAP4 dephosphorylation-related microtubule network densification in pressure overload cardiac hypertrophy. J Biol Chem. 2010; 285(49):38125-40.
58. Fassett J T, Hu X, Xu X, Lu Z, Zhang P, Chen Y, et al. AMPK attenuates microtubule proliferation in cardiac hypertrophy. Am J Physiol Heart Circ Physiol. 2013; 304(5):H749-58.
59. Mangan M E, and Olmsted J B. A muscle-specific variant of microtubule-associated protein 4 (MAP4) is required in myogenesis. Development. 1996; 122(3):771-81.
60. Mogessie B, Roth D, Rahil Z, and Straube A. A novel isoform of MAP4 organises the paraxial microtubule array required for muscle cell differentiation. Elife. 2015; 4:e05697.
61. Teng M, Dang Y M, Zhang J P, Zhang Q, Fang Y D, Ren J, et al. Microtubular stability affects cardiomyocyte glycolysis by HIF-lalpha expression and endonuclear aggregation during early stages of hypoxia. Am J Physiol Heart Circ Physiol. 2010; 298(6):H1919-31.
62. Scholz D, McDermott P, Garnovskaya M, Gallien T N, Huettelmaier S, DeRienzo C, et al. Microtubule-associated protein-4 (MAP-4) inhibits microtubule-dependent distribution of mRNA in isolated neonatal cardiocytes. Cardiovasc Res. 2006; 71(3):506-16.
63. Scholz D, Baicu C F, Tuxworth W J, Xu L, Kasiganesan H, Menick D R, et al. Microtubule-dependent distribution of mRNA in adult cardiocytes. Am J Physiol Heart Circ Physiol. 2008; 294(3):H1135-44.
64. Banmeyer I, Marchand C, Clippe A, and Knoops B. Human mitochondrial peroxiredoxin 5 protects from mitochondrial DNA damages induced by hydrogen peroxide. FEBS Lett. 2005; 579(11):2327-33.
65. Dubuisson M, Vander Stricht D, Clippe A, Etienne F, Nauser T, Kissner R, et al. Human peroxiredoxin 5 is a peroxynitrite reductase. FEBS Lett. 2004; 571(1-3):161-5.
66. Aguero J, Ishikawa K, Hadri L, Santos-Gallego C, Fish K, Hammoudi N, et al. Characterization of right ventricular remodeling and failure in a chronic pulmonary hypertension model. Am J Physiol Heart Circ Physiol. 2014; 307(8):H1204-15.
67. Gregori M, Tocci G, Giammarioli B, Befani A, Ciavarella G M, Ferrucci A, et al. Abnormal regulation of renin angiotensin aldosterone system is associated with right ventricular dysfunction in hypertension. Can J Cardiol. 2014; 30(2):188-94.
68. Gregori M, Giammarioli B, Tocci G, Befani A, Ciavarella G M, Ferrucci A, et al. Synergic effects of renin and aldosterone on right ventricular function in hypertension: a tissue Doppler study. J Cardiovasc Med (Hagerstown). 2015; 16(12):831-8.
69. Maron B A, Opotowsky A R, Landzberg M J, Loscalzo J, Waxman A B, and Leopold J A. Plasma aldosterone levels are elevated in patients with pulmonary arterial hypertension in the absence of left ventricular heart failure: a pilot study. Eur J Heart Fail. 2013; 15(3):277-83.
70. Safdar Z, Thakur A, Singh S, Ji Y, Guffey D, Minard C G, et al. Circulating Aldosterone Levels and Disease Severity in Pulmonary Arterial Hypertension. J Pulm Respir Med. 2015; 5(5).
71. Kang R, Zeh H J, Lotze M T, and Tang D. The Beclin 1 network regulates autophagy and apoptosis. Cell Death Differ. 2011; 18(4):571-80.
72. Tanida I, Ueno T, and Kominami E. LC3 and Autophagy. Methods Mol Biol. 2008; 445:77-88.
73. Codogno P, Mehrpour M, and Proikas-Cezanne T. Canonical and non-canonical autophagy: variations on a common theme of self-eating? Nat Rev Mol Cell Biol. 2012; 13(1):7-12.
74. Lazarou M, Sliter D A, Kane L A, Sarraf S A, Wang C, Burman J L, et al. The ubiquitin kinase PINK1 recruits autophagy receptors to induce mitophagy. Nature. 2015; 524(7565):309-14.
75. Dai D F, Johnson S C, Villarin J J, Chin M T, Nieves-Cintron M, Chen T, et al. Mitochondrial oxidative stress mediates angiotensin II-induced cardiac hypertrophy and Galphaq overexpression-induced heart failure. Circulation research. 2011; 108(7):837-46.
76. Dai D F, and Rabinovitch P. Mitochondrial oxidative stress mediates induction of autophagy and hypertrophy in angiotensin-II treated mouse hearts. Autophagy. 2011; 7(8):917-8.
77. Lee J, Giordano S, and Zhang J. Autophagy, mitochondria and oxidative stress: cross-talk and redox signalling. Biochem J. 2012; 441(2):523-40.
78. Garofalo T, Matarrese P, Manganelli V, Marconi M, Tinari A, Gambardella L, et al. Evidence for the involvement of lipid rafts localized at the ER-mitochondria associated membranes in autophagosome formation. Autophagy. 2016; 12(6):917-35.
79. Hein S, Arnon E, Kostin S, Schonburg M, Elsasser A, Polyakova V, et al. Progression from compensated hypertrophy to failure in the pressure-overloaded human heart: structural deterioration and compensatory mechanisms. Circulation. 2003; 107(7):984-91.
80. Kostin S, Pool L, Elsasser A, Hein S, Drexler H C, Arnon E, et al. Myocytes die by multiple mechanisms in failing human hearts. Circulation research. 2003; 92(7):715-24.
81. Matsui Y, Takagi H, Qu X, Abdellatif M, Sakoda H, Asano T, et al. Distinct roles of autophagy in the heart during ischemia and reperfusion: roles of AMP-activated protein kinase and Beclin 1 in mediating autophagy. Circulation research. 2007; 100(6):914-22.
82. Nakai A, Yamaguchi O, Takeda T, Higuchi Y, Hikoso S, Taniike M, et al. The role of autophagy in cardiomyocytes in the basal state and in response to hemodynamic stress. Nat Med. 2007; 13(5):619-24.
83. Xu X, Kobayashi S, Chen K, Timm D, Volden P, Huang Y, et al. Diminished autophagy limits cardiac injury in mouse models of type 1 diabetes. J Biol Chem. 2013; 288(25):18077-92.
84. Zhu H, Tannous P, Johnstone J L, Kong Y, Shelton J M, Richardson J A, et al. Cardiac autophagy is a maladaptive response to hemodynamic stress. J Clin Invest. 2007; 117(7):1782-93.
85. Fu L, Wei C C, Powell P C, Bradley W E, Collawn J F, and Dell'Italia L J. Volume overload induces autophagic degradation of procollagen in cardiac fibroblasts. J Mol Cell Cardiol. 2015; 89(Pt B):241-50.
86. Ucar A, Gupta S K, Fiedler J, Erikci E, Kardasinski M, Batkai S, et al. The miRNA-212/132 family regulates both cardiac hypertrophy and cardiomyocyte autophagy. Nat Commun. 2012; 3:1078.
87. Shirakabe A, Zhai P, Ikeda Y, Saito T, Maejima Y, Hsu C P, et al. Drp1-Dependent Mitochondrial Autophagy Plays a Protective Role Against Pressure Overload-Induced Mitochondrial Dysfunction and Heart Failure. Circulation. 2016; 133(13): 1249-63.
88. Essick E E, Wilson R M, Pimentel D R, Shimano M, Baid S, Ouchi N, et al. Adiponectin modulates oxidative stress-induced autophagy in cardiomyocytes. PLoS One. 2013; 8(7):e68697.
89. Wu P, Yuan X, Li F, Zhang J, Zhu W, Wei M, et al. Myocardial Upregulation of Cathepsin D by Ischemic Heart Disease Promotes Autophagic Flux and Protects Against Cardiac Remodeling and Heart Failure. Circ Heart Fail. 2017; 10(7).
90. Fletcher K, Ulferts R, Jacquin E, Veith T, Gammoh N, Arasteh J M, et al. The WD40 domain of ATG16L1 is required for its non-canonical role in lipidation of LC3 at single membranes. EMBO J. 2018; 37(4).
91. Su H, Yang F, Wang Q, Shen Q, Huang J, Peng C, et al. VPS34 Acetylation Controls Its Lipid Kinase Activity and the Initiation of Canonical and Non-canonical Autophagy. Mol Cell. 2017; 67(6):907-21 e7.
92. Codogno P, Mehrpour M, and Proikas-Cezanne T. Canonical and non-canonical autophagy: variations on a common theme of self-eating? Nat Rev Mol Cell Biol. 2011; 13(1):7-12.
93. Proikas-Cezanne T, Takacs Z, Donnes P, and Kohlbacher O. WIPI proteins: essential PtdIns3P effectors at the nascent autophagosome. J Cell Sci. 2015; 128(2):207-17.
94. Itakura E, Kishi-Itakura C, Koyama-Honda I, and Mizushima N. Structures containing Atg9A and the ULK1 complex independently target depolarized mitochondria at initial stages of Parkin-mediated mitophagy. J Cell Sci. 2012; 125(Pt 6): 1488-99.
95. Muller A J, and Proikas-Cezanne T. Function of human WIPI proteins in autophagosomal rejuvenation of endomembranes? FEBS Lett. 2015; 589(14):1546-51.
96. Grotemeier A, Alers S, Pfisterer S G, Paasch F, Daubrawa M, Dieterle A, et al. AMPK-independent induction of autophagy by cytosolic Ca2+ increase. Cell Signal. 2010; 22(6):914-25.
97. Pfisterer S G, Mauthe M, Codogno P, and Proikas-Cezanne T. Ca2+/calmodulin-dependent kinase (CaMK) signaling via CaMKI and AMP-activated protein kinase contributes to the regulation of WIPI-1 at the onset of autophagy. Mol Pharmacol. 2011; 80(6): 1066-75.
98. Horvath S, Zhang B, Carlson M, Lu K V, Zhu S, Felciano R M, et al. Analysis of oncogenic signaling networks in glioblastoma identifies ASPM as a molecular target. Proc Natl Acad Sci USA. 2006; 103(46):17402-7.
99. Zhang B, and Horvath S. A general framework for weighted gene co-expression network analysis. Stat Appl Genet Mol Biol. 2005; 4:Article 17.
100. Xue Z, Huang K, Cai C, Cai L, Jiang C Y, Feng Y, et al. Genetic programs in human and mouse early embryos revealed by single-cell RNA sequencing. Nature. 2013; 500(7464):593-7.
101. Duan H, Ge W, Zhang A, Xi Y, Chen Z, Luo D, et al. Transcriptome analyses reveal molecular mechanisms underlying functional recovery after spinal cord injury. Proc Natl Acad Sci USA. 2015; 112(43):13360-5.
102. Luo Y, Coskun V, Liang A, Yu J, Cheng L, Ge W, et al. Single-cell transcriptome analyses reveal signals to activate dormant neural stem cells. Cell. 2015; 161(5):1175-86.
103. Liu Q, Jiang C, Xu J, Zhao M T, Van Bortle K, Cheng X, et al. Genome-Wide Temporal Profiling of Transcriptome and Open Chromatin of Early Cardiomyocyte Differentiation Derived From hiPSCs and hESCs. Circulation research. 2017; 121(4):376-91.
104. Dipla K, Mattiello J A, Jeevanandam V, Houser S R, and Margulies K B. Myocyte recovery after mechanical circulatory support in humans with end-stage heart failure. Circulation. 1998; 97(23):2316-22.
105. Atluri P, Goldstone A B, Fairman A S, MacArthur J W, Shudo Y, Cohen J E, et al. Predicting right ventricular failure in the modern, continuous flow left ventricular assist device era. Ann Thorac Surg. 2013; 96(3):857-63; discussion 63-4.
106. Kormos R L, Teuteberg J J, Pagani F D, Russell S D, John R, Miller L W, et al. Right ventricular failure in patients with the HeartMate II continuous-flow left ventricular assist device: incidence, risk factors, and effect on outcomes. J Thorac Cardiovasc Surg. 2010; 139(5): 1316-24.
107. Ben-Ari Fuchs S, Lieder I, Stelzer G, Mazor Y, Buzhor E, Kaplan S, et al. GeneAnalytics: An Integrative Gene Set Analysis Tool for Next Generation Sequencing, RNAseq and Microarray Data. OMICS. 2016; 20(3):139-51.
108. Assenov Y, Ramirez F, Schelhorn S E, Lengauer T, and Albrecht M. Computing topological parameters of biological networks. Bioinformatics. 2008; 24(2):282-4.
109. Csardi G, and Nepusz T. The igraph software package for complex network research. http://igraph.org. Updated 2006 Accessed 1695.
110. Fagerberg L, Hallstrom B M, Oksvold P, Kampf C, Djureinovic D, Odeberg J, et al. Analysis of the human tissue-specific expression by genome-wide integration of transcriptomics and antibody-based proteomics. Mol Cell Proteomics. 2014; 13(2):397-406.
111. Shannon P, Markiel A, Ozier O, Baliga N S, Wang J T, Ramage D, et al. Cytoscape: a software environment for integrated models of biomolecular interaction networks. Genome Res. 2003; 13(11):2498-504.
112. Warnes G R, Bolker B, Bonebakker L, Gentleman R, Huber W, Liaw A, et al. gplots: Various R Programming Tools for Plotting Data. R package version 3.0.1. 2016:https://CRAN.R-project.org/package=gplots.
113. Tarnayski O, McMullen J R, Schinke M, Nie Q, Kong S, and Izumo S. Mouse cardiac surgery: comprehensive techniques for the generation of mouse models of human diseases and their application for genomic studies. Physiol Genomics. 2004; 16(3):349-60.
114. Tsai E J, Liu Y, Koitabashi N, Bedja D, Danner T, Jasmin J F, et al. Pressure-overload-induced subcellular relocalization/oxidation of soluble guanylyl cyclase in the heart modulates enzyme stimulation. Circulation research. 2012; 110(2):295-303.
115. Kohut A, Patel N, and Singh H. Comprehensive Echocardiographic Assessment of the Right Ventricle in Murine Models. J Cardiovasc Ultrasound. 2016; 24(3):229-38.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A method for treating or ameliorating the effect of a cardiopulmonary disease in a subject, comprising modulating the expression of at least one gene of a gene module associated with right ventricular failure (RVF) in the subject.

2. The method of claim 1, wherein the gene module comprises the following genes: WIPI1, HSPB6, MAP4, SNAP47, and PRDX.

3. The method of claim 1, wherein the modulation comprises decreasing the expression of at least one of WIPI1, HSPB6, MAP4, and SNAP47, and/or increasing the expression of PRDX, in the subject.

4. The method of claim 1, wherein the modulation comprises decreasing the expression of WIPI1, HSPB6, and MAP4, in the subject.

5. The method of claim 1, wherein the modulation comprises decreasing the expression of WIPI1, in the subject.

6. The method of claim 1, wherein the cardiopulmonary disease is associated with right ventricular failure (RVF).

7. The method of claim 1, wherein the cardiopulmonary disease is selected from heart failure and pulmonary hypertension.

8. A method for diagnosing right ventricular failure (RVF) in a subject, comprising:
(a) obtaining a biological sample from the subject;
(b) determining the expression level of at least one gene of a gene module in the sample and comparing it to a reference determined in a healthy subject;
(c) diagnosing the subject as being at risk for right ventricular failure (RVF) if the expression level of the at least one gene of the gene module in the sample is significantly higher than the reference; and
(d) initiating a treatment protocol for the subject diagnosed in step (c) as being at risk for RVF.

9. The method of claim 8, wherein the gene module comprises the following genes: WIPI1, HSPB6, MAP4.

10. The method of claim 8, wherein the at least one gene is WIPI1.

11. The method of claim 8, wherein the treatment protocol comprises modulating WIPI1 expression.

12. A method for preventing right ventricular failure (RVF) in a subject, comprising decreasing the expression of WIPI1, in the subject.

13. The method of claim 12, wherein the subject has at least one of the following: right ventricular dysfunction (RVD), reduced ejection fraction, preserved ejection fraction, a left ventricular assist device, pulmonary hypertension, and cardiovascular etiology.

14. A method for preventing non-canonical autophagy in a cardiac myocyte, comprising decreasing the expression of WIPI1, in the cardiac myocyte.

15. The method of claim 14, wherein the non-canonical autophagy is induced by a neurohormone.

16. The method of claim 15, wherein the neurohormone is aldosterone.

17. A method for mitigating oxidative stress in mitochondria of a cardiac myocyte, comprising decreasing the expression of WIPI1, in the cardiac myocyte.

18. The method of claim 17, wherein the oxidative stress is aldosterone-induced.

19. The method of claim 17, wherein the oxidative stress is not induced by hydrogen peroxide.

20. A pharmaceutical composition comprising: a first vector expressing CRISPR associated protein 9 (CAS9), a second vector expressing WIPI1 gRNA, and a pharmaceutically acceptable carrier.

21. A method for treating or ameliorating the effect of a cardiopulmonary disease in a subject, comprising administering to the subject a therapeutically effective amount of the pharmaceutical composition according to claim 20.


SEQUENCE LISTING (34 sequences)

SEQ ID NO: 1 - DNA, 22 bp, Artificial Sequence
  Forward Primer
ctttcaaccaagactgcacatc

SEQ ID NO: 2 - DNA, 20 bp, Artificial Sequence
  Reverse Primer
gttcatctgccgaggttttg

SEQ ID NO: 3 - DNA, 22 bp, Artificial Sequence
  Forward Primer
gctcctttaccaggtttctctg

SEQ ID NO: 4 - DNA, 22 bp, Artificial Sequence
  Reverse Primer
atccagcagcacggaaaaatac

SEQ ID NO: 5 - DNA, 22 bp, Artificial Sequence
  Forward Primer
tttaccgggtttttccactccg

SEQ ID NO: 6 - DNA, 21 bp, Artificial Sequence
  Reverse Primer
cttcacatccagcagcacaga

SEQ ID NO: 7 - DNA, 21 bp, Artificial Sequence
  Forward Primer
ccccaaagaaacagagacaac

SEQ ID NO: 8 - DNA, 19 bp, Artificial Sequence
  Reverse Primer
ctgagagtgaaaccatgcc

SEQ ID NO: 9 - DNA, 19 bp, Artificial Sequence
  Forward Primer
ctcctctctgccctctccc

SEQ ID NO: 10 - DNA, 20 bp, Artificial Sequence
  Reverse Primer
ccgccattctttaccactgc

SEQ ID NO: 11 - DNA, 20 bp, Artificial Sequence
  Forward Primer
ggagctgacacagatcctga

SEQ ID NO: 12 - DNA, 20 bp, Artificial Sequence
  Reverse Primer
catacgccggttttgcttgt

SEQ ID NO: 13 - DNA, 19 bp, Artificial Sequence
  Forward Primer
cttctgcgcgctcctgttg

SEQ ID NO: 14 - DNA, 20 bp, Artificial Sequence
  Reverse Primer
aggccaggtgtgaactcgta

SEQ ID NO: 15 - DNA, 20 bp, Artificial Sequence
  Forward Primer
tggcctgtctgagcgttaat

SEQ ID NO: 16 - DNA, 20 bp, Artificial Sequence
  Reverse Primer
gagaaccttttcagccgacg

SEQ ID NO: 17 - DNA, 18 bp, Artificial Sequence
  Forward Primer
actatggccccgatcaag

SEQ ID NO: 18 - DNA, 20 bp, Artificial Sequence
  Reverse Primer
ggaacagccaggtgtaaatg

SEQ ID NO: 19 - DNA, 22 bp, Artificial Sequence
  Forward Primer
gtgacagtggtaaaggcaaagg

SEQ ID NO: 20 - DNA, 20 bp, Artificial Sequence
  Reverse Primer
tcagattttcccggtggaga

SEQ ID NO: 21 - DNA, 20 bp, Artificial Sequence
  Forward Primer
gcagcaagaaggaccagacc

SEQ ID NO: 22 - DNA, 22 bp, Artificial Sequence
  Reverse Primer
tttcccaaatcgagaggagttg

SEQ ID NO: 23 - DNA, 20 bp, Artificial Sequence
  Forward Primer
gcttcgggggtaggattgac

SEQ ID NO: 24 - DNA, 20 bp, Artificial Sequence
  Reverse Primer
tagatgaaggcaggaagccg

SEQ ID NO: 25 - DNA, 20 bp, Artificial Sequence
  Forward Primer
cttcggtctcaaggcagcac

SEQ ID NO: 26 - DNA, 21 bp, Artificial Sequence
  Reverse Primer
gagacccaggcagagtcagaa

SEQ ID NO: 27 - DNA, 20 bp, Artificial Sequence
  Forward Primer
agcctcacttcctaccctcg

SEQ ID NO: 28 - DNA, 20 bp, Artificial Sequence
  Reverse Primer
ttgtcacacacaagagcggt

SEQ ID NO: 29 - DNA, 20 bp, Artificial Sequence
  Forward Primer
gcaccttgagaggaacagaa

SEQ ID NO: 30 - DNA, 22 bp, Artificial Sequence
  Reverse Primer
gagcacccgcttagtcttatag

SEQ ID NO: 31 - DNA, 21 bp, Artificial Sequence
  Forward Primer
ttctccatcacctacaaaccc

SEQ ID NO: 32 - DNA, 20 bp, Artificial Sequence
  Reverse Primer
accagtctttattggcctcg

SEQ ID NO: 33 - DNA, 21 bp, Artificial Sequence
  Forward Primer
gttctccatcacctacaagcc

SEQ ID NO: 34 - DNA, 21 bp, Artificial Sequence
  Reverse Primer
acgagtctttattgtccccac